(19) United States
(12) Patent Application Publication
(10) Pub. No.: US 2025/0304983 A1
(43) Pub. Date: Oct. 2, 2025

(54) TRANSCRIPTION ACTIVATORS AND PROGRAMMABLE TRANSCRIPTION ENGINEERING

(71) Applicant: Regents of the University of Minnesota, Minneapolis, MN (US)

(72) Inventors: Matthew Harley Zinselmeier, Minneapolis, MN (US); Michael Joseph Smanski, Minneapolis, MN (US); Daniel F. Voytas, Minneapolis, MN (US)

(21) Appl. No.: 18/862,329

(22) PCT Filed: May 2, 2023
(86) PCT No.: PCT/US2023/020643

§ 371 Date: Nov. 1, 2024

Related U.S. Application Data
(60) Provisional application No. 63/337,402, filed on May 2, 2022.

(51) Int. Cl. C12N 15/82; C07K 16/00; C12N 9/22; C12N 15/113; C12N 15/62

(52) U.S. Cl. CPC C12N 15/8216; C07K 16/00; C12N 9/222; C12N 15/113; C12N 15/62; C07K 2317/569; C07K 2317/622; C07K 2319/71; C07K 2319/81

(57) ABSTRACT

Methods and materials for stimulating expression of target genes in plants are provided herein.

### CROSS-REFERENCE TO RELATED APPLICATIONS

This application claims priority from U.S. Provisional Application Ser. No. 63/337,402, filed May 2, 2022. The disclosure of the prior application is considered part of (and is incorporated by reference in) the disclosure of this application.

### STATEMENT AS TO FEDERALLY SPONSORED RESEARCH

This invention was made with government support under 2018-33522-28747 awarded by the National Institute of Food and Agriculture, USDA. The government has certain rights in the invention.

This invention was made with government support under DE-SC0018277 awarded by the Dept. of Energy. The government has certain rights in the invention.

### SEQUENCE LISTING

This application contains a Sequence Listing that has been submitted electronically as an XML file named “09531-0505WO1_ST26.XML.” The XML file, created on May 1, 2023, is 509,789 bytes in size. The material in the XML file is hereby incorporated by reference in its entirety.

### TECHNICAL FIELD

This document relates to methods and materials for stimulating expression of target genes in plants.

### BACKGROUND

Controlling the expression of endogenous genes in plants can be done to obtain information related to plant development. Plant genomes provide the blueprint for growth, survival, and reproduction. In order to survive, a plant must respond to environmental conditions such as temperature, light, or humidity. Gene expression is the process by which the genetic blueprint is put into action, activating genes at different times and in different tissues to develop, reproduce, and survive in the face of external stimuli (Klepikova et al., Plant J., 88(6):1058-1070, 2016; and Knauer et al., Genome Res., 29(12):1962-1973, 2019). The ability to correlate differential gene expression with phenotype can facilitate the identification of genes that are important for proper development and response to environmental stimuli (Pang et al., Mol Plant., 13(9):1311-1327, 2020; and He et al., Nature Commun., 13(1):1-15, 2022).

### SUMMARY

Programmable Transcription Activators (PTAs) are fusion polypeptides or polypeptide systems in which a transcription activation domain (AD) is coupled directly or indirectly to a DNA-binding domain that can be engineered to recognize a DNA sequence of interest (e.g., the promoter region and/or an enhancer region of a gene). For example, PTAs containing a dCas9 polypeptide fused to VP64 (a tetrameric repeat of the VP16 protein derived from herpes simplex virus; Sadowski et al., Nature 335(6190):563-564, 1988; and Beerli et al., Proc Natl Acad Sci USA., 95(25):14628-14633, 1998) can activate expression of target genes in plants, as described elsewhere (Lowder et al., Mol Plant., 11(2):245-256, 2018). This document is based, at least in part, on the identification of plant-derived ADs that can function as well or better than VP64 for PTA applications in plants.

This document provides methods and materials for targeted stimulation of gene expression in plants. For example, this document provides fusion polypeptides that contain an AD and a DNA-binding domain that can target the AD to a particular sequence (e.g., a sequence in or near a promoter region of a gene of interest). As demonstrated herein, about 40 sequences from plant genomes were identified as potentially having the ability to function as ADs when fused to a programmable DNA binding domain. The library was tested in transient protoplast assays, and a set of ADs with strong transcription activation ability as compared to negative and positive controls was identified. In particular, the AvrXa10 (TALE-derived) (SEQ ID NO:84), Dof1 (Zea mays transcription factor MNB1A) (SEQ ID NO:76), and DREB2 (Arabidopsis thaliana DRE-binding protein 2A) (SEQ ID NO:78) ADs demonstrated on average about 3-fold higher activation ability than the VP64 positive control. The AvrXa10, Dof1, and DREB2 ADs functioned in a variety of contexts—fused directly to dCas9, fused to a single-chain fragment variable (scFv) polypeptide in the dCas9-SunTag system, and fused to a scFv in a TALE-SunTag system. In addition, Dof1 and DREB2 were consistently strong ADs across multiple species that included both monocots and dicots.

In a first aspect, this document features a polypeptide containing, consisting essentially of, or consisting of an AD and a DNA-binding domain, where the AD and the DNA-binding domain are not naturally present within the same protein, and where the AD contains an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98. For example, the AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The DNA-binding domain can include a dCas polypeptide. The dCas polypeptide can be a dCas9 polypeptide (e.g., a dCas9 polypeptide having an amino acid sequence with at least 95% identity to the amino acid sequence set forth in SEQ ID NO:119). The DNA-binding domain can contain a transcription activator-like effector (TALE) polypeptide or a zinc finger binding domain.

In another aspect, this document features a transcriptional activator system. The transcriptional activator system can include, consist of, or consist essentially of, first and second fusion polypeptides, where the first fusion polypeptide contains (a) an AD portion and (b) a single-chain fragment variable (scFv) portion or a nanobody portion, where the AD portion contains an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98, and where the second fusion polypeptide contains (a) a DNA-binding domain and (b) one or more scFv or nanobody binding sequences. The AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The first fusion polypeptide can include a scFv portion and the second fusion polypeptide can contain one or more scFv binding sequences. The scFv portion can include an amino acid sequence with at least 95% sequence identity to the amino acid sequence set forth in SEQ ID NO:123, and the one or more scFv binding sequences can include an amino acid sequence with at least 94% sequence identity to the amino acid sequence set forth in SEQ ID NO:121. The first fusion polypeptide can include a nanobody portion and the second fusion polypeptide can include a nanobody binding sequence. The nanobody portion can include an amino acid sequence having at least 97% sequence identity to the amino acid sequence set forth in SEQ ID NO:122, and the one or more nanobody binding sequences can include an amino acid sequence with at least 93% sequence identity with the amino acid sequence set forth in SEQ ID NO:120. The second fusion polypeptide can include ten or more scFv or nanobody binding sequences. The DNA-binding domain can include a dCas polypeptide. The dCas polypeptide can be a dCas9 polypeptide having an amino acid sequence with at least 95% identity to the amino acid sequence set forth in SEQ ID NO:119. The transcriptional activator system can further include a single guide RNA (sgRNA). The -binding domain can include a TALE polypeptide or a zinc finger binding domain. The first fusion polypeptide can further include a solubility tag. The solubility tag can include immunoglobulin-binding domain of protein G (GB1), super folding green fluorescent protein (sfGFP), or both GB1 and sfGFP.

In another aspect, this document features a nucleic acid containing a nucleotide sequence encoding an AD and a DNA-binding domain, where the AD and the DNA-binding domain are not naturally present within the same protein, and where the encoded AD contains an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98. For example, the AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The DNA-binding domain can include a dCas polypeptide. The dCas polypeptide can be a dCas9 polypeptide having an amino acid sequence with at least 95% identity to the amino acid sequence set forth in SEQ ID NO:119. The DNA-binding domain can include a TALE polypeptide or a zinc finger binding domain.

In another aspect, this document features a transcriptional activator system containing, consisting of, or consisting essentially of: a nucleic acid encoding a first fusion polypeptide that includes (a) an AD portion and (b) a scFv portion or a nanobody portion, where the AD portion contains an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and a nucleic acid encoding a second fusion polypeptide that includes (a) a DNA-binding domain and (b) one or more scFv or nanobody binding sequences. The AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The first fusion polypeptide can include a scFv portion and the second fusion polypeptide can include one or more scFv binding sequences. The scFv portion can include an amino acid sequence with at least 95% sequence identity to the amino acid sequence set forth in SEQ ID NO:123, and the one or more scFv binding sequences can include an amino acid sequence with at least 94% sequence identity to the amino acid sequence set forth in SEQ ID NO:121. The first fusion polypeptide can include a nanobody portion and the second fusion polypeptide can include a nanobody binding sequence. The nanobody portion can contain an amino acid sequence having at least 97% sequence identity to the amino acid sequence set forth in SEQ ID NO:122, and the one or more nanobody binding sequences can contain an amino acid sequence with at least 93% sequence identity with the amino acid sequence set forth in SEQ ID NO:120. The second fusion polypeptide can include ten or more scFv or nanobody binding sequences. The DNA-binding domain can contain a dCas polypeptide. The dCas polypeptide can be a dCas9 polypeptide having an amino acid sequence with at least 95% identity to the amino acid sequence set forth in SEQ ID NO:119. The transcriptional activator system can further include a nucleic acid encoding a sgRNA. The DNA-binding domain can include a TALE polypeptide or a zinc finger binding domain. The first fusion polypeptide can further contain a solubility tag. The solubility tag can include GB1, sfGFP, or both GB1 and sfGFP.

In another aspect, this document features a method for activating transcription of a target gene in a plant cell. The method can include or consist essentially of: introducing a nucleic acid molecule into a plant cell, wherein the nucleic acid molecule contains a nucleotide sequence encoding a fusion polypeptide that includes an AD and a DNA-binding domain targeted to the gene, where the AD and the DNA-binding domain are not naturally present within the same protein, and where the encoded AD contains an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and allowing the cell to express the fusion polypeptide, such that the fusion polypeptide activates transcription of the gene. The AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The DNA-binding domain can include a dCas polypeptide, and the method can further include introducing into the plant cell a nucleic acid encoding a single guide RNA (sgRNA). The dCas polypeptide can be a dCas9 polypeptide having an amino acid sequence with at least 95% identity to the amino acid sequence set forth in SEQ ID NO:119. The DNA-binding domain can include a TALE polypeptide or a zinc finger binding domain. The DNA-binding domain can be targeted to a promoter sequence of the gene. In some cases, the method can further include introducing a second nucleic acid molecule into the plant cell, where the second nucleic acid molecule contains a nucleotide sequence encoding a second fusion polypeptide that contains a second AD and a second DNA-binding domain targeted to the gene, where the second AD and the second DNA-binding domain are not naturally present within the same protein, where the second AD contains an amino acid sequence with at least 95% sequence identity to SEQ ID NO:76, SEQ ID NO:78, SEQ ID NO:84, SEQ ID NO:77, SEQ ID NO:92, SEQ ID NO:95, or SEQ ID NO:98, and the second DNA-binding domain can be targeted to a potential or previously identified enhancer sequence of the gene.

In still another aspect, this document features a method for activating transcription of a target gene in a plant cell, where the method includes or consists essentially of (1) introducing a transcriptional activator system into a plant cell, where the transcriptional activator system includes a nucleic acid encoding a first fusion polypeptide that contains (a) an AD portion and (b) a scFv portion or a nanobody portion, where the AD portion includes an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and a nucleic acid encoding a second fusion polypeptide containing (a) a DNA-binding domain and (b) one or more scFv or nanobody binding sequences; and (2) allowing the cell to express the first and second fusion polypeptides, such that transcription of the gene is activated. The AD can contain an amino acid sequence with at least 95% sequence identity to the amino acid sequence set forth in SEQ ID NO:76, the amino acid sequence set forth in SEQ ID NO:76, an amino acid sequence with at least 95% sequence identity to the amino acid sequence set forth in SEQ ID NO:78, the amino acid sequence set forth in SEQ ID NO:78, an amino acid sequence with at least 95% sequence identity to the amino acid sequence set forth in SEQ ID NO:84, the amino acid sequence set forth in SEQ ID NO:84, an amino acid sequence with at least 95% sequence identity to the amino acid sequence set forth in SEQ ID NO:77, the amino acid sequence set forth in SEQ ID NO:77, an amino acid sequence with at least 95% sequence identity to the amino acid sequence set forth in SEQ ID NO:92, the amino acid sequence set forth in SEQ ID NO:92, an amino acid sequence with at least 95% sequence identity to the amino acid sequence set forth in SEQ ID NO:95, the amino acid sequence set forth in SEQ ID NO:95, an amino acid sequence with at least 95% sequence identity to the amino acid sequence set forth in SEQ ID NO:98, or the amino acid sequence set forth in SEQ ID NO:98. The DNA-binding domain can include a dCas polypeptide, and the method can further include introducing into the plant cell a nucleic acid encoding a sgRNA. The dCas polypeptide can be a dCas9 polypeptide having an amino acid sequence with at least 95% identity to the amino acid sequence set forth in SEQ ID NO:119. The DNA-binding domain can include a TALE polypeptide or a zinc finger binding domain. The first fusion polypeptide can include a scFv portion and the second fusion polypeptide can include one or more scFv binding sequences. The scFv portion can contain an amino acid sequence with at least 95% sequence identity to the amino acid sequence set forth in SEQ ID NO:123, and the one or more scFv binding sequences can contain an amino acid sequence with at least 94% sequence identity to the amino acid sequence set forth in SEQ ID NO:121. The first fusion polypeptide can contain a nanobody portion and the second fusion polypeptide can contain a nanobody binding sequence. The nanobody portion can include an amino acid sequence having at least 97% sequence identity to the amino acid sequence set forth in SEQ ID NO:122, and the one or more nanobody binding sequences can include an amino acid sequence with at least 93% sequence identity with the amino acid sequence set forth in SEQ ID NO:120. The second fusion polypeptide can contain ten or more scFv or nanobody binding sequences. The first fusion polypeptide can further contain a solubility tag. The solubility tag can include GB1, sfGFP, or both GB1 and sfGFP. The DNA-binding domain can be targeted to a promoter sequence of the gene. The transcriptional activator system can further include: a nucleic acid encoding a third fusion polypeptide that contains (a) a second AD portion and (b) a scFv portion or a nanobody portion, where the second AD portion includes an amino acid sequence with at least 95% sequence identity to SEQ ID NO:76, SEQ ID NO:78, SEQ ID NO:84, SEQ ID NO:77, SEQ ID NO:92, SEQ ID NO:95, or SEQ ID NO:98; and a nucleic acid encoding a fourth fusion polypeptide containing (a) a second DNA-binding domain and (b) one or more scFv or nanobody binding sequences, where the second DNA-binding domain is targeted to a potential or previously identified enhancer sequence of the gene.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A-1G: Screening a library of plant-derived activation domains with the dual luciferase protoplast assay. FIG. 1A is a schematic of a dCas9-based Programmable Transcription Activator (PTA) including a direct fusion of an AD to dCas9. FIG. 1B is a schematic of a dCas9-based PTA in a SunTag scaffolding system. FIG. 1C is a schematic of a dCas9-based PTA in a MoonTag scaffolding system. Genetic construct design for the plasmids transformed into Setaria viridis or Arabidopsis thaliana protoplasts are shown below the schematics in FIGS. 1A-1C. FIG. 1D shows a phylogenetic tree indicating the diversity of sequences tested in the studies described herein. The multiple sequence alignment and phylogenetic tree were generated in MEGA using full length amino acid sequences. A Maximum Likelihood tree was generated using 500 bootstraps, with values >0.05 displayed above a given node. Strong activation domains from the assays are boxed on the tree, while the positive control VP16 is circled. FIG. 1E includes a schematic of a single-plasmid dual luciferase reporter construct (top), a graph plotting the correlation of PTA-inducible Renilla luciferase activity vs. “constitutive” Firefly luciferase activity (bottom). FIG. 1F includes a schematic of the dual luciferase reporters on separate constructs (top), and a graph plotting the correlation of PTA-inducible Renilla luciferase activity vs. “constitutive” Firefly luciferase activity (bottom). FIG. 1G is a graph plotting a rank order of the strongest ADs for S. viridis protoplasts against the VP64 positive control. Parametric t-tests were performed comparing each AD with the negative control (−)sgRNA. ns, not significant; *p≤0.05; **p≤0.01; ***p≤0.001 FIG. 2: Direct fusion PTA vs SunTag PTA comparison in A. thaliana protoplasts.

FIG. 2 is a graph plotting gene activation in protoplasts transformed with direct fusion class PTAs containing dCas9-VP64 and dCas9-TV (which includes six copies of the TAL activation domain and two copies of the VP64 activation domain translationally fused to dCas9) along with a 4× Wuschel sgRNA array targeting the core promoter for gene activation. RNA was extracted and activation was quantified by RT-qPCR using the delta Ct method. Parametric t-tests were performed comparing dCas9-TV or SunTag-VP64 with the dCas9-VP64 direct fusion at the same locus. *p≤0.05; **p≤0.01.

FIG. 3: 35S Batch One S. viridis Dual Luciferase Assay. The graph in FIG. 3 shows luciferase reporter activity in a first protoplast isolation for S. viridis transformed with a library of scFv-ADs and the single dual luciferase vector. Expression of Renilla luciferase was driven by a 35S promoter. Data were fit to a linear regression model, with R and p values indicated in the graph.

FIG. 4: 35S Batch Two S. viridis Dual Luciferase Assay. The graph in FIG. 4 shows luciferase reporter activity in a second protoplast isolation for S. viridis transformed with a library of scFv-ADs and the single dual luciferase vector. Expression of Renilla luciferase was driven by a 35S promoter. Data were fit to a linear regression model, with R and p values in the graph.

FIG. 5: A. thaliana Split Dual Luciferase Assay. The graph in FIG. 5 shows the fold change in activity after a smaller library of scFv-ADs was transformed into A. thaliana protoplasts along with the split dual luciferase vectors. The Firefly luciferase vector contained the 6×LacO-mini35S promoter to drive luciferase expression, while the Renilla luciferase vector contained a constitutive 35S promoter to drive luciferase expression. Firefly luciferase relative luminescence units (RLUs) were divided by Renilla luciferase RLUs to normalize for transformation. The scFv-AD normalized RLUs were then divided by the scFv-VP64 normalized RLU to generate the graph. A parametric t-test was performed to compare the negative control (−) sgRNA with each respective AD. ns, not significant; *p≤0.05; **p≤0.01; ***p≤0.001.

FIG. 6: S. viridis Single Dual Luciferase Assay. The full library of scFv-ADs was transformed into S. viridis protoplasts along with the single dual luciferase vectors. The single luciferase reporter contained the Firefly luciferase driven by the 6×LacO-mini35S promoter, while the Renilla luciferase was driven by the constitutive 35S promoter. Firefly luciferase RLUs were compared to the positive control VP64. A parametric t-test was performed to compare the negative control (−) sgRNA with each respective AD. ns, not significant; *p≤0.05; **p≤0.01; ***p≤0.001. In addition to the ADs shown, non-significant results also were obtained for BRE1, Opaque2, SNAC1, LBD2, EFS, VRN1, AtARF8, ATI4, AtHSFA2, Asf1b, GT-3a, CVBp12, HAG1, bHLH122, MYC2, ATXR7, OsCBT, Asf1a, Ada2b, and ABI5.

FIG. 7: A. thaliana Single Dual Luciferase Assay. The full library of scFv-ADs was transformed into A. thaliana protoplasts along with the single dual luciferase vectors. The single luciferase reporter contained the Firefly luciferase driven by the 6×LacO-mini35S promoter, while the Renilla luciferase was driven by the constitutive 35S promoter. Firefly luciferase RLUs were compared to the positive control VP64. A parametric t-test was performed comparing the negative control (−) sgRNA with each respective AD. ns, not significant; *p≤0.05; **p≤0.01. In addition to the ADs shown, non-significant results also were obtained for EFS, OsGRF1, OsCBT, SDG31, ABI5, ATXR7, Asf1a, Ada2b, PTI4, LMI1, CVBp12, BRE1, and Asf1b.

FIG. 8: Zea mays Split Dual Luciferase Assay. Greened Z. mays plants were used for protoplast isolation and transformation. A smaller library of scFv-ADs was transformed into Z. mays protoplasts along with split dual luciferase vectors. The Firefly luciferase vector contained the 6×LacO-mini35S promoter, while the Renilla luciferase vector contained the constitutive 35S promoter. Firefly luciferase RLUs were divided by Renilla luciferase RLUs to normalize for transformation. The scFv-AD normalized RLUs were then divided by the scFv-VP64 normalized RLU to generate the graph. ns, not significant; **p≤0.01.

FIGS. 9A-9E: Endogenous gene activation in A. thaliana protoplasts. FIG. 9A is a schematic showing guide RNA design for a given gene promoter, indicating the sgRNA distance to the known transcription start site (TSS) for the Flowering Locus T (FT), Clavata3 (Clv3), Production of Anthocyanin Pigment 1 (PAP1), and Wuschel (WUS) loci. FIG. 9B is a graph plotting activation data for each of the indicated genes, shown relative to the housekeeping gene PP2A. FIG. 9C is a graph plotting aggregate gene activation across all genes tested, where each dot represents an independent protoplast transformation. FIG. 9D includes a schematic of a TALE-SunTag system in which a TALE DNA binding domain was fused to the GCN4 epitope tail (top), as well as a schematic illustrating the TALE DNA binding domain targeted to the Lec1 promoter as compared to dCas9 binding to the same promoter (bottom). FIG. 9E is a graph plotting activation of the LEC1 target gene in A. thaliana by dCas9-SunTag or TALE-SunTag. Parametric t-tests were performed to compare each AD with the negative control, noAD. ns, not significant; *p≤0.05; ***p≤0.001; ****p 0.0001.

FIG. 10: A. thaliana protoplast Clv3 gene activation with gRNA1. Protoplasts were isolated from A. thaliana and transformed with Ubi10-dCas9-24xGCN, 35S-scFv-AD, and AtU6-Clv3g1. RNA was isolated from cells 24 hours after transformation, and RT-qPCR was performed to quantify gene expression. The delta delta Ct method was used to calculate fold change vs. the NoAD negative control. PP2a was used as the housekeeping gene. ns, not significant; *p≤0.05; **p≤0.01; ***p≤0.001.

FIG. 11: A. thaliana protoplast Clv3 gene activation with gRNA2. Protoplasts were isolated from A. thaliana and transformed with Ubi10-dCas9-24xGCN, 35S-scFv-AD, and AtU6-Clv3g2. RNA was isolated from cells 24 hours post-transformation, and RT-qPCR was performed to quantify gene expression. The delta delta Ct method was used to calculate fold change vs. the NoAD negative control. PP2a was used as the housekeeping gene. ns, not significant; *p≤0.05.

FIG. 12: A. thaliana protoplast FT gene activation with FTgA. Protoplasts were isolated from A. thaliana and transformed with Ubi10-dCas9-24xGCN, 35S-scFv-AD, and AtU6-FTgA. RNA was isolated from cells 24 hours post-transformation, and RT-qPCR was performed to quantify gene expression. The delta delta Ct method was used to calculate fold change vs. the NoAD negative control. PP2a was used as the housekeeping gene. ns, not significant.

FIGS. 13A-13F: FT gene activation in transgenic plants. FIG. 13A is a diagram showing a genetic construct design for T-DNAs floral dipped into transgenic A. thaliana plants. FIG. 13B is a diagram of a construct for targeting the FT core promoter with two sgRNAs, each binding within 200 bp of the transcription start site. FIG. 13C is a representative image of T2 transgenic plants for an AvrXa10 transgenic line. The wildtype Col-0 plant is shown on the left, the positive control VP64 line is shown in the middle, and the AvrXa10 plant for the transgenic line is shown on the right. FIG. 13D is a graph plotting the molecular quantification of FT gene activation from 5 day-old T2 seedlings. FIG. 13E is a graph plotting bolting time in T2 transgenic plants. FIG. 13F is a graph plotting rosette leaf quantification one day after bolting in T2 transgenic plants. Parametric t-tests were performed to compare each line with the negative control Col-0. **p≤0.01; ***p≤0.001; ****p≤0.0001.

FIG. 14: T1 A. thaliana Parental Lines. FIG. 14 includes representative images showing T1 parental lines and an age-matched wild type Col-0 A. thaliana plant. The TDNA vectors used for these plants were identical, except for the AD fused to scFv. The label below each image denotes the AD in each parental line.

FIGS. 15A-15D: PTA-mediated enhancer activation at the Arabidopsis FT locus. FIG. 15A is a diagram depicting the regions targeted by guide RNAs (gRNAs) designed to target regions at varying distances from the known FT TSS. The FT Core promoter (−197 bp), Block B (−1.1 kb), Block C (−5.3 kb), and Block E (+2.9 kb) enhancers were targeted, as were intervening sequences equidistant from a given enhancer and its nearest transcriptional regulatory element. FIGS. 15B-15D are graphs plotting FT gene activation when targeting Block C (FIG. 15B), Block B (FIG. 15C) or Block E (FIG. 15D) with different combinations of gRNAs. RNA was extracted 24 hours post transformation, and FT gene activation was quantified via RT-qPCR. Parametric t-tests were performed to compare each AD with a negative control in which no gRNA was delivered. ns, not significant; *p≤0.05; **p≤0.01.

FIG. 16 is a graph plotting the level of non-specific activity for various ADs in S. viridis protoplasts. The non-specific activity of each strong plant-derived AD was measured by transforming S. viridis protoplasts with the indicated scFv-AD fusion and dual luciferase reporter plasmid. A non-parametric t-test was performed to compare the negative control (VP64 alone) with each individual scFv-AD. In the positive control (VP64+ dCas9), the dual luciferase, sgRNA, dCas9, and scFv-VP64 were co-transformed into S. viridis protoplasts.

FIGS. 17A-17F are graphs plotting the level of off-target gene activation in A. thaliana protoplasts. The off-target activity of strong plant-derived ADs (VP64, DREB1, DREB2, HSFA6b, and Dof1) was quantified using RT-qPCR for downstream target genes of the endogenous transcription factors. A non-parametric t-test was performed to compare downstream gene expression for a given AD with the negative control (NoAD) in which protoplasts were transformed with dCas9-24xGCN4 and sgRNA, without the AD plasmid. Downstream target genes included rd29a (FIG. 17A), kin1 (FIG. 17B), lea7 (FIG. 17C), cel3 (FIG. 17D), pme18 (FIG. 17E), and dreb2a (FIG. 17F), and are listed in TABLE 6 along with their associated ADs. The on-target guide in these experiments was the sgRNA targeting the WUS promoter region.

FIG. 18 is a graph plotting the level of off-target activation of the FAS1 gene in A. thaliana protoplasts. The off-target activity of FAS1 was quantified by RT-qPCR in A. thaliana protoplasts. The FT locus was targeted with sgRNAs to activate both the core promoter and nearby enhancers Block B, Block C, and Block E; the on-target guides are denoted below the graph. Little FAS1 expression change was measured when comparing across groups using a non-parametric t-test to compare each treatment with the FT Core+No AD negative control.

### DETAILED DESCRIPTION

This document provides PTA polypeptides that contain an AD coupled directly to a DNA-binding domain (e.g., in a fusion polypeptide) or coupled indirectly to a DNA-binding domain (e.g., via fusion with a polypeptide that interacts with the DNA-binding domain), where the DNA-binding domain can be engineered to recognize a DNA sequence of interest. In addition, this document provides methods for using the PTAs provided herein to increase the expression of targeted genes in plants. The strength of overexpression driven by the PTAs provided herein is strongly correlated to a target gene's basal expression levels. As described herein and elsewhere (Chiarella et al., Nature Biotechnol., 38(1):50-55, 2020), PTAs targeted to genes normally expressed at low levels can achieve higher fold-overexpression values than PTAs targeted to highly-expressed genes.

### Polypeptides

In one aspect, this document provides PTA polypeptides containing an AD and a DNA-binding domain. The term “polypeptide” as used herein refers to a compound of two or more subunit amino acids, regardless of post-translational modification (e.g., phosphorylation or glycosylation). The subunits may be linked by peptide bonds or other bonds such as, for example, ester or ether bonds. The term “amino acid” refers to either natural and/or unnatural or synthetic amino acids, including D/L optical isomers.

By “isolated” or “purified” with respect to a polypeptide it is meant that the polypeptide is separated to some extent from cellular components with which it normally is found in nature (e.g., other polypeptides, lipids, carbohydrates, and nucleic acids). A purified polypeptide can yield a single major band on a non-reducing polyacrylamide gel. A purified polypeptide can be at least about 75% pure (e.g., at least 80%, 85%, 90%, 95%, 97%, 98%, 99%, or 100% pure). Purified polypeptides can be obtained by, for example, extraction from a natural source, by chemical synthesis, or by recombinant production in a host cell or transgenic plant, and can be purified using, for example, affinity chromatography, immunoprecipitation, size exclusion chromatography, and ion exchange chromatography. The extent of purification can be measured using any appropriate method, including, without limitation, column chromatography, polyacrylamide gel electrophoresis, or high-performance liquid chromatography.

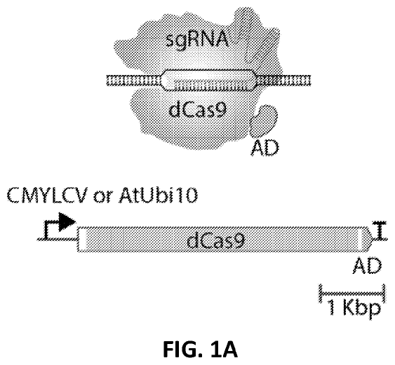

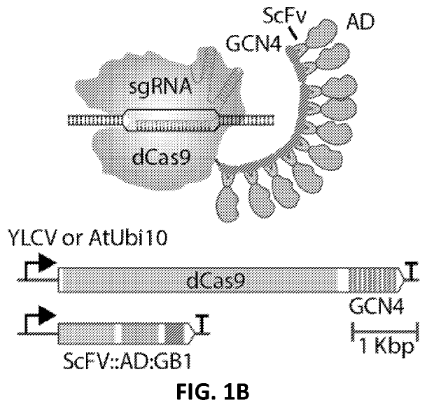

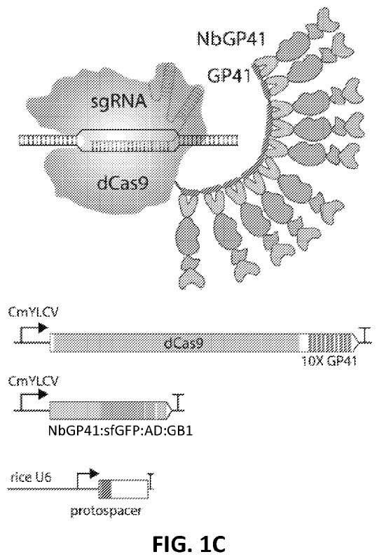

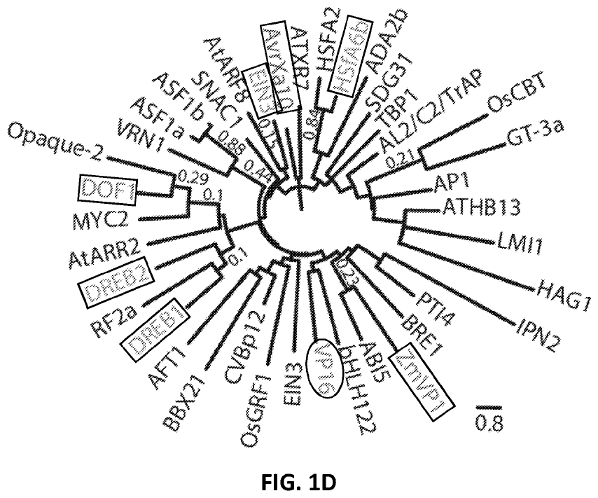

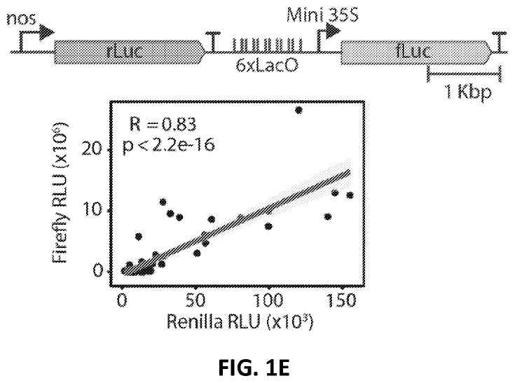

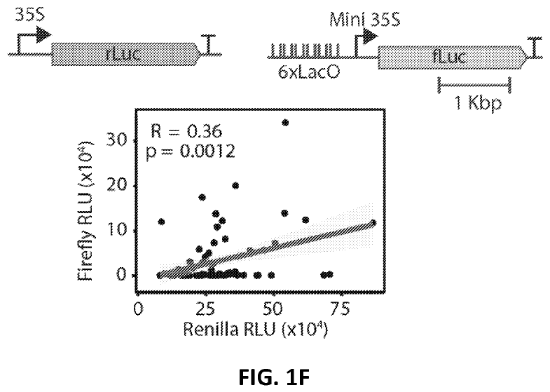

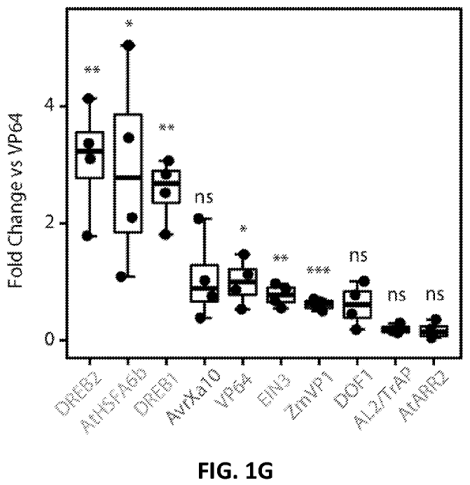

In some cases, the AD and the DNA-binding domain of a PTA provided herein are included a single fusion polypeptide, such that they are encoded by one nucleotide sequence and are expressed as a single polypeptide driven by one promoter. The AD can be N-terminal to the DNA-binding domain, or the AD can be C-terminal to the DNA-binding domain (e.g., as illustrated in FIG. 1A).

In some cases, the AD and the DNA-binding domain can be present in separate polypeptides, such that they are encoded by separate nucleotide sequences (e.g., on separate constructs) and may be expressed from different promoters. When the AD and the DNA-binding domain are expressed as separate polypeptides, the AD can be recruited to the DNA-binding domain. For example, the SunTag system uses a non-covalent antigen-antibody interaction between (1) a single-chain variable fragment antibody (scFv) fused to an AD, and (2) a tandemly-repeated 19-amino acid epitope tail (GCN4 motif) fused to a DNA-binding domain (e.g., dCas9) to recruit multiple copies of the AD to the DNA-binding domain via the GCN4 repeats, as illustrated in FIG. 1B and described elsewhere (Tanenbaum et al., Cell, 159(3):635-646, 2014). The MoonTag system is another example of a system in which the AD and the DNA-binding domain are separate polypeptides. In the MoonTag system, the scFv-GCN4 interaction of SunTag is replaced with a nanobody-peptide (e.g., NbGP41-GP41) interaction to recruit the AD to the DNA-binding domain (e.g., as illustrated in FIG. 1C).

Any appropriate AD can be included in a PTA provided herein. For example, a PTA can include an AD derived from a plant protein, such as the ADs listed in TABLE 4 herein.

In some cases, the PTA can include a plant-derived AD from a DREB2 protein (e.g., A. thaliana DREB2). An example of an AD derived from DREB2 is set forth in SEQ ID NO:78. Thus, in some cases, a PTA provided herein can include one or more copies of an AD having the amino acid sequence set forth in SEQ ID NO:78, or having an amino acid sequence with at least 95% (e.g., at least 96%, at least 97%, at least 98%, or at least 99%) sequence identity to SEQ ID NO:78.

(SEQ ID NO: 78)
SSDMFDVDELLRDLNGDDVFAGLNQDRYPGNSVANGSYRPESQQSGFDP
LQSLNYGIPPFQLEGKDGNGFFDDLSYLDLEN

In some cases, the PTA can include a plant-derived AD from a Dof1 protein (e.g., Z. mays Dof1). An example of an AD derived from Dof1 is set forth in SEQ ID NO:76. In some cases, a PTA provided herein can include one or more copies of an AD having the amino acid sequence set forth in SEQ ID NO:76, or having an amino acid sequence with at least 95% (e.g., at least 96%, at least 97%, at least 98%, or at least 99%) sequence identity to SEQ ID NO:76.

(SEQ ID NO: 76)
QPGTEDAEAVALGLGLSDFPSAGKAVLDDEDSFVWPAASFDMGACWAGA
GFADPDPACIFLNLP

In some cases, the PTA can include a plant-derived AD from a AvrXa10 protein. An example of an AD derived from AvrXa10 is set forth in SEQ ID NO:84. Thus, in some cases, a PTA provided herein can include one or more copies of an AD having the amino acid sequence set forth in SEQ ID NO:84, or having an amino acid sequence with at least 95% (e.g., at least 96%, at least 97%, at least 98%, or at least 99%) sequence identity to SEQ ID NO:84.

(SEQ ID NO: 84)
TVMWEQDAAPFAGAADDFPAFNEEELAWLMELLPQSGSVGGTI

In some cases, the PTA can include a plant-derived AD from a DREB1 protein (e.g., A. thaliana DREB1). An example of an AD derived from DREB1 is set forth in SEQ ID NO:77. Thus, in some cases, a PTA provided herein can include one or more copies of an AD having the amino acid sequence set forth in SEQ ID NO:77, or having an amino acid sequence with at least 95% (e.g., at least 96%, at least 97%, at least 98%, or at least 99%) sequence identity to SEQ ID NO:77.

(SEQ ID NO: 77)
GRSACLNFADSAWRLRIPESTCAKDIQKAAAEAALAFQDEMCDATTDHGF
DMEETLVEAIYTAEQSENAFYMHDEAMFEMPSLLANMAEGMLLPLPSVQW
NHNHEVDGDDDDVSLWSY

In some cases, the PTA can include a plant-derived AD from a ZmVP1 protein. An example of an AD derived from ZmVP1 is set forth in SEQ ID NO:92. Thus, in some cases, a PTA provided herein can include one or more copies of an AD having the amino acid sequence set forth in SEQ ID NO:92, or having an amino acid sequence with at least 95% (e.g., at least 96%, at least 97%, at least 98%, or at least 99%) sequence identity to SEQ ID NO:92.

(SEQ ID NO: 92)
MEASSGSSPPHSQENPPEHGGDMGGAPAEEIGGEAADDFMFAEDTFPSLP
DFPCLSSPSSSTFSSNSSSNSSSAYTNTAGRAGGEPSEPASAGEGFDALD
DIDQLLDFASLSMPWDSEPF

In some cases, the PTA can include a plant-derived AD from an AtHSFA6b protein. An example of an AD derived from AtHSFA6b is set forth in SEQ ID NO:95. Thus, in some cases, a PTA provided herein can include one or more copies of an AD having the amino acid sequence set forth in SEQ ID NO:95, or having an amino acid sequence with at least 95% (e.g., at least 96%, at least 97%, at least 98%, or at least 99%) sequence identity to SEQ ID NO:95.

(SEQ ID NO: 95)
KEKRKEIEEAISKKRQRPIDQGKRNVEDYGDESGYGNDVAASSSALIGMS
QEYTYGNMSEFEMSELDKLAMHIQGLGDNSSAREEVLNVEKGNDEEEVED
QQQGYHKENNEIYGEGFWEDLLNEGQNFDFEGDQENVDVGSSSHTN

In some cases, the PTA can include a plant-derived AD from a EIN3 protein. An example of an AD derived from EIN3 is set forth in SEQ ID NO:98. Thus, in some cases, a PTA provided herein can include one or more copies of an AD having the amino acid sequence set forth in SEQ ID NO:98, or having an amino acid sequence with at least 95% (e.g., at least 96%, at least 97%, at least 98%, or at least 99%) sequence identity to SEQ ID NO:98.

(SEQ ID NO: 98)
SLSGGSCSLLMNDCSQYDVEGFEKESHYEVEELKPEKVMNSSNFGMVAKM
HDFPVKEEVPAGNSEFMRKRKPNRDLNTIMDRTVFTCENLGCAHSEISRG
FLDRNSRDNHQLACPHRDSRLPYGAAPSRFHVNEVKPVVGFPQPRPVNSV
AQPIDLTGIVPEDGQKMISELMSMYDRNVQSNQTSMVMENQSVSLLQPTV
HNHQEHLQFPGNMVEGSFFEDLNIPNRANNNNSSNNQTFFQGNNNNNNVF
KFDTADHNNFEAAHNNNNNSSGNRFQLVFDSTPFDMASFDYRDDMSMPGV
VGTMDGMQQKQQDVSIWF

The percent sequence identity between a particular amino acid or nucleic acid sequence and an amino acid or nucleic acid sequence referenced by a particular sequence identification number is determined as follows. First, an amino acid or nucleic acid sequence is compared to the sequence set forth in a particular sequence identification number using the BLAST 2 Sequences (Bl2seq) program from the stand-alone version of BLASTZ containing BLASTN version 2.0.14 and BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two sequences using either the BLASTN or BLASTP algorithm. BLASTN is used to compare nucleic acid sequences, while BLASTP is used to compare amino acid sequences. To compare two nucleic acid sequences, the options are set as follows: -i is set to a file containing the first nucleic acid sequence to be compared (e.g., C:\seq1.txt); -j is set to a file containing the second nucleic acid sequence to be compared (e.g., C:\seq2.txt); -p is set to blastn; -o is set to any desired file name (e.g., C:\output.txt); -q is set to −1; -r is set to 2; and all other options are left at their default setting. For example, the following command can be used to generate an output file containing a comparison between two sequences: C:Bl2seq -i c:\seq1.txt -j c:\seq2.txt -p blastn -o c:\output.txt -q −1 -r 2. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq -i c:\seq1.txt -j c:\seq2.txt -p blastp -o c:\output.txt. If the two compared sequences share homology, then the designated output file will present those regions of homology as aligned sequences. If the two compared sequences do not share homology, then the designated output file will not present aligned sequences.

Once aligned, the number of matches is determined by counting the number of positions where an identical nucleotide or amino acid residue is presented in both sequences. A matched position refers to a position in which an identical nucleotide or amino acid residue occurs at the same position in aligned sequences. The percent sequence identity is determined by dividing the number of matches by the length of the sequence set forth in the identified sequence (e.g., SEQ ID NO:78), or by an articulated length (e.g., 20 consecutive nucleotides or amino acid residues from a sequence set forth in an identified sequence), followed by multiplying the resulting value by 100. For example, an amino acid sequence that has 77 matches when aligned with the sequence set forth in SEQ ID NO:78 is 95.1 percent identical to the sequence set forth in SEQ ID NO:78 (i.e., 77÷81×100=95.1). It is noted that the percent sequence identity value is rounded to the nearest tenth. For example, 75.11, 75.12, 75.13, and 75.14 are rounded down to 75.1, while 75.15, 75.16, 75.17, 75.18, and 75.19 are rounded up to 75.2. It also is noted that the length value will always be an integer.

In some cases, a portion of the amino acid sequence of a PTA provided herein (e.g., the AD portion, the DNA-binding portion, the scFV binding portion, or the nanobody binding portion) can contain one or more conservative substitutions as compared to a representative amino acid sequence for that portion of the PTA (e.g., SEQ ID NO:76 or SEQ ID NO:78, which are representative ADs). A conservative substitution for an amino acid in a polypeptide can be selected from other members of the class to which the amino acid belongs. For example, an amino acid belonging to a group of amino acids having a particular size or characteristic (such as charge, hydrophobicity and hydrophilicity) can be substituted for another amino acid within the same group without altering the activity of a protein, particularly in regions of the protein that are not directly associated with biological activity. For example, nonpolar (hydrophobic) amino acids include alanine, leucine, isoleucine, valine, proline, phenylalanine, tryptophan, and tyrosine. Polar neutral amino acids include glycine, serine, threonine, cysteine, tyrosine, asparagine, and glutamine. Positively charged (basic) amino acids include arginine, lysine, and histidine. Negatively charged (acidic) amino acids include aspartic acid and glutamic acid. Conservative substitutions include, for example, Lys for Arg and vice versa to maintain a positive charge; Glu for Asp and vice versa to maintain a negative charge; Ser for Thr so that a free hydroxyl group is maintained; and Gln for Asn to maintain a free amino group. For example, one or both of the Ser residues in the first and second positions of SEQ ID NO:78 could be replaced with Thr residues, and/or the Asn residue in the last position of SEQ ID NO:78 could be replaced with a Gln residue. With regard to SEQ ID NO:76, the Gln at position 1 could be replaced with Asn, the Pro at position 2 could be replaced with Ala, Leu, Ile, or Val, the Ser at position 4 could be replaced with Thr, the Asn at position 62 could be replaced with Gln, the Leu at position 63 could be replaced with Ala, Val, or Ile, and/or the Pro at position 64 could be replaced with Ala, Leu, Ile, or Val. Without being bound by a particular mechanism, it is noted that the Asp, Glu, Phe, and Trp residues within the AD regions provided herein, as well as the Leu and Val residues flanking the Asp, Glu, Phe, and Trp residues, are generally retained. In addition, it is noted that biologically active analogs of a polypeptide containing deletions or additions of one or more contiguous or noncontiguous amino acids that do not eliminate a functional activity of the polypeptide are also contemplated.

The PTAs provided herein can contain any appropriate DNA-binding domain. In some cases, for example, the DNA-binding domain can be a zinc-finger DNA binding domain (Ji et al., Nucl Acids Res. 42(10):6158-6167, 2014). In some cases, the DNA-binding domain can be a transcription activator-like effector (TALE) DNA-binding domain (Lowder et al., supra). Further, in some cases, the DNA-binding domain can be a catalytically “dead” Cas protein (dCas) that is directed to a target sequence via a single guide RNA (sgRNA) (Casas-Mollano et al., CRISPR J., 3(5):350-364, 2020). PTAs containing dCas9 can, in some cases, target multiple sequences in parallel by co-expressing them with multiple sgRNAs (Zhou et al., Nature Neurosci. 21(3):440-446, 2018). Any appropriate dCas protein can be used. Examples of dCas polypeptides proteins include, without limitation, Cas3, Cas8, Cas10, Cas9, Cas12, and Cas13. In some cases, the dCas polypeptide can be dCas9. A representative example of a dCas9 amino acid sequence is set forth in SEQ ID NO:119. In some cases, the dCas protein can a part of a larger protein complex.

When the DNA-binding domain in a PTA provided herein is a dCas polypeptide, the PTA also includes a sgRNA designed to recognize and bind to a target DNA sequence. The sgRNA can complex with the dCas polypeptide, thus directing the dCas to the target sequence. For example, a sgRNA can be designed to bind to a target DNA sequence in or near a promoter region, a transactivation region, or an enhancer region. The sgRNA can be encoded by a nucleotide sequence that is present on the same construct as the sequence encoding the DNA-binding domain and/or the AD, or the sgRNA can be encoded by a nucleotide sequence that is on a separate construct.

In some cases, the DNA-binding domain can be included in a fusion polypeptide with one or more copies of a scFv or a nanobody binding polypeptide. Each scFv or nanobody binding polypeptide can include an amino acid sequence that provides a binding interface with a scFv or a nanobody. In some cases, the fusion polypeptide can include two or more (e.g., three, four, five, six, seven, eight, nine, ten, 11, 12, 13, 14, 15, 16, 17, 18 19, 20, or more than 20) copies of the scFv or nanobody binding polypeptide. The fusion polypeptide can include two or more of the same scFv or nanobody binding amino acid sequence, or the fusion polypeptide can include two or more different scFv or nanobody binding sequences. In some cases, the scFv or nanobody binding polypeptide can include at least two of the same scFv or nanobody binding sequence and at least one different scFv or nanobody binding sequence. In some cases, the scFv or nanobody binding polypeptide can be GP41 (SEQ ID NO:120) or GCN4 (SEQ ID NO:121), or a polypeptide having at least 93% (e.g., at least 94%, at least 95%, at least 96%, at least 97%, at least 98%, or at least 99%) amino acid sequence identity with SEQ ID NO: 120 or SEQ ID NO:121).

When a PTA provided herein includes a DNA-binding domain that is part of a fusion polypeptide with one or more copies of a scFv or a nanobody binding polypeptide, the PTA also can include a second fusion polypeptide that contains an AD and a scFv or a nanobody, such that interaction of the scFv or the nanobody with the scFv or nanobody binding polypeptide(s) of the first fusion polypeptide containing the DNA-binding domain will recruit the AD to the DNA-binding domain, and thus to the targeted DNA sequence. A representative nanobody sequence is set forth in SEQ ID NO:122, and a representative scFv sequence is set forth in SEQ ID NO:123. In some cases, a nanobody can have an amino acid sequence with at least 95% (e.g., at least 95%, at least 96%, at least 97%, at least 98%, or at least 99%) with the amino acid sequence set forth in SEQ ID NO:122, which can interact with a polypeptide having the amino acid sequence of SEQ ID NO:120. In some cases, a scFv can have an amino acid sequence with at least 95% (e.g., at least 95%, at least 96%, at least 97%, at least 98%, or at least 99%) with the amino acid sequence set forth in SEQ ID NO:123, which can interact with a polypeptide having the amino acid sequence of SEQ ID NO:121.

In some cases, the region of a fusion polypeptide containing two or more copies of a scFv or nanobody binding polypeptide can include a spacer amino acid sequence between the DNA-binding polypeptide and the closest scFv or nanobody binding polypeptide (referred to as the “N-terminal spacer sequence”), and/or between adjacent copies of the scFv or nanobody binding sequences. A spacer amino acid sequence can contain from about 5 to about 25 amino acids. In some cases, the spacer amino acid sequence can be GSGSG (SEQ ID NO:124). In some cases, the spacer amino acid sequences between each pair of adjacent scFV or nanobody binding sequences can be the same. In some cases, the spacer amino acid sequences between each pair of adjacent scFv or nanobody binding sequences can differ from one another. In some cases, at least two spacer amino acid sequences between can be the same, and at least one spacer amino acid sequence can be different from the first two.

When present, the N-terminal spacer sequence (between the DNA-binding polypeptide and the first scFv or nanobody binding sequence) can be the same as or different from the spacer amino acid sequences between adjacent scFv or nanobody binding sequences. In some cases, for example, the N-terminal spacer sequence can be longer than the other amino acid spacer sequences in the polypeptide. In some cases, the N-terminal spacer sequence can be shorter than the amino acid spacer sequence. In some cases, the N-terminal spacer amino acid sequence can be GSGSG (SEQ ID NO:124). In some cases, the N-terminal spacer amino acid sequence can include a nuclear localization signal.

In some cases, a fusion polypeptide containing an AD and a scFv or a nanobody also can include one or more (e.g., two, three, four, five, or more than five) solubility tags. Any appropriate solubility tag can be used, provided that the tag does not reduce or destroy the ability of the scFv or the nanobody to bind to scFv or nanobody amino acid binding sequence(s) and does not reduce or destroy the ability of the AD to activate transcription. Examples of suitable solubility tags include, without limitation, immunoglobulin-binding domain of protein G (GB1; SEQ ID NO:125), super folding green fluorescent protein (sfGFP; SEQ ID NO:126), glutathione-S-transferase (GST (SEQ ID ON:127), thioredoxin (Trx; SEQ ID NO:128), small ubiquitin-related modifier (SUMO; SEQ ID NO: 129), maltose/maltodextrin ABC transporter substrate-binding protein (MBP; SEQ ID NO:130), and FLAG-tag (SEQ ID NO:131, optionally repeated one or two times; see, e.g., Costa et al., Front Microbiol., 5:63, 2014). In some cases, the solubility tag can be GB1 (SEQ ID NO:125) or sfGFP (SEQ ID NO:126). When the solubility tag is sfGFP, the tag also can provide a visible signal. Other tags that can be used to provide a visible signal include, without limitation, RFP and mCherry.

FIG. 1C illustrates an exemplary MoonTag activating system that includes a DNA binding component (in this case, dCas9) and an AD component. As illustrated, the dCas9 DNA-binding domain (e.g., SEQ ID NO:119) is fused to a nanobody binding polypeptide that includes ten copies of the binding amino acid sequence GP41 (dCas9-10XGP41). The DNA binding component of the MoonTag activation system also includes a sgRNA, which as illustrated is expressed from a separate construct. The AD component includes a GP41 nanobody fused to sfGFP, GB1, and an AD (as illustrated, dCas9). When expressed in plant cells, the dCas9-10XGP41 fusion binds to its target region guided by the sgRNA. The region of the DNA-binding component that includes the GP41 nanobody binding sequences is bound by the GP41 nanobody, thus recruiting up to ten copies of the AD to the ribonucleoprotein complex.

### Nucleic Acids

This document also provides nucleic acid molecules containing sequences that encode the PTA polypeptides described herein. The terms “nucleic acid” and “polynucleotide” can be used interchangeably, and refer to both RNA and DNA, including cDNA, genomic DNA, synthetic (e.g., chemically synthesized) DNA, and DNA (or RNA) containing nucleic acid analogs. Polynucleotides can have any three-dimensional structure. A nucleic acid can be double-stranded or single-stranded (i.e., a sense strand or an antisense single strand). Non-limiting examples of polynucleotides include genes, gene fragments, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers, as well as nucleic acid analogs.

The fusion polypeptides described herein can be provided to plant cells by introducing one or more vectors encoding the polypeptide(s) to be used, for example. As used herein, “isolated,” when in reference to a nucleic acid, refers to a nucleic acid that is separated from other nucleic acids that are present in a genome, e.g., a plant genome, including nucleic acids that normally flank one or both sides of the nucleic acid in the genome. The term “isolated” as used herein with respect to nucleic acids also includes any non-naturally-occurring sequence, since such non-naturally-occurring sequences are not found in nature and do not have immediately contiguous sequences in a naturally-occurring genome.

An isolated nucleic acid can be, for example, a DNA molecule, provided one of the nucleic acid sequences normally found immediately flanking that DNA molecule in a naturally-occurring genome is removed or absent. Thus, an isolated nucleic acid includes, without limitation, a DNA molecule that exists as a separate molecule (e.g., a chemically synthesized nucleic acid, or a cDNA or genomic DNA fragment produced by PCR or restriction endonuclease treatment) independent of other sequences, as well as DNA that is incorporated into a vector, an autonomously replicating plasmid, a virus (e.g., a pararetrovirus, a retrovirus, lentivirus, adenovirus, or herpes virus), or the genomic DNA of a prokaryote or eukaryote. In addition, an isolated nucleic acid can include a recombinant nucleic acid such as a DNA molecule that is part of a hybrid or fusion nucleic acid. A nucleic acid existing among hundreds to millions of other nucleic acids within, for example, cDNA libraries or genomic libraries, or gel slices containing a genomic DNA restriction digest, is not to be considered an isolated nucleic acid.

A nucleic acid can be made by, for example, chemical synthesis or polymerase chain reaction (PCR). The nucleic acids provided herein can be incorporated into or contained within recombinant nucleic acid constructs such as vectors. A “vector” is a replicon, such as a plasmid, phage, or cosmid, into which another DNA segment may be inserted so as to bring about the replication of the inserted segment. Generally, a vector is capable of replication when associated with the proper control elements. Suitable vector backbones include, for example, those routinely used in the art such as plasmids, viruses, artificial chromosomes, BACs, YACs, or PACs. The term “vector” includes cloning and expression vectors, as well as viral vectors and integrating vectors. An “expression vector” is a vector that includes one or more “expression control sequences” that control or regulate the transcription and/or translation of another DNA sequence. Suitable expression vectors include, without limitation, plasmids and viral vectors derived from, for example, bacteriophage, baculoviruses, tobacco mosaic virus, herpes viruses, cytomegalovirus, retroviruses, vaccinia viruses, adenoviruses, and adeno-associated viruses. Numerous vectors and expression systems are commercially available from such corporations as Novagen (Madison, WI), Clontech (Palo Alto, CA), Stratagene (La Jolla, CA), and Invitrogen/Life Technologies (Carlsbad, CA).

In some cases, the nucleic acids provided herein can include nucleotide sequences encoding, for example, fusion polypeptides in which a DNA-binding domain (e.g., a zinc finger nuclease, TALE, or dCas DNA-binding polypeptide) is coupled directly to an AD. In some cases, the nucleic acids provided herein can include nucleotide sequences encoding fusion polypeptides in which a DNA-binding domain is coupled to one or more scFv or nanobody binding polypeptides, with or without an additional amino acid sequence (e.g., one or more spacer sequences). In some cases, the nucleic acids provided herein can include nucleotide sequences encoding fusion polypeptides in which an AD is coupled to a scFv or a nanobody, with or without an additional amino acid sequences (e.g., one or more spacer sequences, solubility tags, or sequences that, when expressed, produce a visible signal).

The sequences encoding the fusion polypeptides in the nucleic acids provided herein can be operably linked to any appropriate promoters, such that the promoter effectively controls expression of the polypeptide coding sequences. In addition, the sequences encoding sgRNAs for use in the systems provided herein can be operably linked to any appropriate promoter, such that the promoter effectively controls expression of the sgRNAs. In some cases, a promoter operably linked to a nucleotide sequence encoding an sgRNA or a fusion polypeptide provided herein can be a constitutive promoter that leads to expression of an operably linked nucleic acid in most or all tissues of a plant, throughout plant development. In some cases, a promoter operably linked to a nucleotide sequence encoding an sgRNA or a fusion polypeptide provided herein can be an inducible promoter that leads to expression of an operably linked nucleic acid in response to external stimuli such as chemical agents, developmental stimuli, or environmental stimuli. In some cases, a promoter operably linked to a nucleotide sequence encoding an sgRNA or a fusion polypeptide provided herein can be a tissue specific promoter that leads to expression of an operably linked nucleic acid in one or more particular tissues of a plant. Suitable constitutive promoters include, without limitation, the Cauliflower mosaic virus (CaMV) 35S promoter, the Nos promoter, the S. viridis CMYLCV promoter, the A. thaliana Ubi10 (AtUbi10) promoter, the Z. mays Ubi1 (ZmUbi1) promoter, and the O. sativa U6 promoter. Suitable inducible promoters include, for example, heat shock inducible promoters (e.g., hsp70; Ainley and Key, Plant Mol Biol. 14(6):949-967, 1990), and XVE inducible promoters (Zuo et al., Plant J., 24(2):265-273, 2000. Suitable tissue-specific promoters include, for example, phosphoenolpyruvate (PEP) carboxylase (PEPC) and glycine decarboxylase P-protein (GLDP) promoters that drive expression of photosynthesis-related proteins and can lead to expression in leaves (Ermakova, Maria, et al. “Installation of C4 photosynthetic pathway enzymes in rice using a single construct.” Plant Biotechnol. J. 19:575-588, 2021), and the early embryogenesis Rps5a promoter (Kang et al., Nature Plants 4:427-431, 2018).

### Methods

This document also provides methods for using the polypeptides and nucleic acids described herein. For example, this document provides methods that can be used to increase gene expression of an endogenous gene in a plant genome. In some cases, the methods can include introducing, into a plant cell, a fusion polypeptide (or a nucleic acid encoding a fusion polypeptide) that contains a DNA-binding domain targeted to a selected sequence (e.g., a promoter sequence) for a plant gene and an AD to enhance expression of the plant gene. In some cases, the methods provided herein can include introducing, into a plant cell, a combination of fusion polypeptides (or nucleic acid sequences encoding the fusion polypeptides) that, in combination, target a selected sequence (e.g., a promoter sequence) of a plant gene to enhance expression of the plant gene. As described herein, the combination of fusion polypeptides (also referred to as a components of a system) can work together in a SunTag scaffolding system or a in MoonTag scaffolding system.

Any appropriate method can be used to introduce a PTA polypeptide or nucleic acid into a plant cell. For example, the polypeptides and nucleic acids can be introduced into plant cells by injection, direct uptake, projectile bombardment, liposomes, electroporation, or transformation. In some cases, protoplasts can be transformed with one or more nucleic acids encoding a PTA (and, when appropriate, an sgRNA). It is to be noted that protoplasts can be regenerated to yield transgenic plants or plants having an edited genome. In some cases, Agrobacterium delivery methods can be used to introduce a PTA polypeptide or nucleic acid into a plant cell. Such methods include, for example, transient techniques such as leaf infiltration and co-culture/soaking methods, as well as stable transgenesis techniques through floral dip or callus co-culture/regeneration.

The polypeptides and systems provided herein can be used for any suitable application. In some cases, for example, the polypeptides and systems provided herein can be used to identify valuable germplasm via high-throughput screening of numerous loci in a plant genome, which can be accomplished by designing a plurality (e.g., tens, hundreds, or even thousands) of sgRNAs that each are targeted to a different sequence. In combination with an AD described herein (e.g., an AD derived from DREB2, such as SEQ ID NO:78, or an AD derived from Dof1, such as SEQ ID NO:76), multiple native promoters or enhancers can be activated, followed by phenotypic characterization. This would allow for the identification of regulatory genomic regions for traits through observation of a gain of function phenotype—something that can be more difficult to observe with standard CRISPR mutagenesis because indels in promoters or enhancers rarely have phenotypes unless they are large deletions.

In some cases, the PTA polypeptides and systems provided herein can be used to engineer circuits in plant species. For example, synthetic promoters can be designed containing PTA binding sites, such that expression of a given PTA can confer activation of the synthetic promoter. Genetic circuits can then be designed by including PTAs in combination with repressors, allowing for combinatorial PTA or repressor inputs that dictate whether or not the synthetic promoter is expressed. For example, an AND statement would require the simultaneous expression of two PTAs targeting a single promoter in order to observe activation. See, e.g., Tickman et al., Cell Syst. 13(3):215-229, 2022.

In addition, the PTA polypeptides and systems described herein can be used to activate distal cis regulatory elements (CREs) in order to identify putative enhancer sequences, and to subsequently identify the genes regulated by those enhancers. Enhancers are broadly defined as cis regulatory elements located at a long distance (e.g., 1000 nucleotides or more) from the core promoter. In some cases, enhancers can confer tissue specific gene expression (see, e.g., Meng et al., The Plant Cell, 33(6):1997-2014, 2021). Identification and confirmation of novel enhancer sequences can be valuable for understanding plant biology, as these sequences can be critical in conferring desired tissue specific gene expression (Weber et al., Trends Plant Sci., 21(11):974-987, 2016). PTAs can be programmed to target both enhancers and a core promoter. When PTAs are targeted to the core promoter and an enhancer region, transcript abundance can be increased significantly more than when targeting the core promoter alone, or when targeting of the core promoter along with a random non-enhancer sequence. Thus, PTAs can be targeted to the core promoter of a gene of interest along with putative enhancer regions in order to identify enhancers capable of significantly activating the target gene relative to random genomic targets.

In addition, in some cases, the PTA polypeptides and systems described herein can be used to engineer synthetic speciation events through conditional overexpression of a lethal target gene in hybrid mating events. Highly efficient PTAs can be used for Engineered Genetic Incompatibility (EGI) studies in which PTAs are targeted to a gene of interest such that overexpression causes lethality due to target gene activation. This can be followed by genome editing to mutate the sgRNA binding site, yielding an EGI organism in which a PTA is expressed in an edited organism lacking the wild type promoter sequence. Hybridization with a wild type individual can produce offspring containing a wild type target site along with expression of the PTA, resulting in genetic incompatibility specifically in hybrid offspring.

The invention will be further described in the following examples, which do not limit the scope of the invention described in the claims.

### EXAMPLES

### Example 1—Materials and Methods

Plasmid construction. Putative ADs were selected from literature analysis for the potential to recruit transcription initiation machinery in plant cells. DNA binding domains from transcription factors were identified and removed on the basis of sequence conservation as compared to experimentally determined DNA binding domain motifs. Patches of acidic/aromatic residues were identified as core motifs for a prospective AD, and each given sequence was selected to encompass as many of these patches as possible without including the DNA binding domain. Synthetic DNA fragments were then designed and codon optimized based on average codon usage in A. thaliana and Oryza sativa. Fragments were synthesized by Genscript with BsmBI cloning sites flanking the prospective AD. Fragments were cloned into an scFv destination vector with BsaI and BsmBI cloning sites generating compatible overhangs. The assembled plasmids generated an in-frame C terminal fusion of the AD to the scFv. The scFv was driven by the CaMV 35S promoter, and also contained a C terminal fusion to a GB1 solubility tag. The scFv-AD was recruited to targets by dCas9-GCN4. This vector was generated by fusing 24 copies of the GCN4 epitope tag to the C-terminal domain (CTD) of A. thaliana codon-optimized dCas9. The GCN4 repeats were separated from one another by short GS linker sequences. The dCas9 was expressed under the AtUbi10 promoter in dicots, the CMYLCV promoter in S. viridis, or the ZmUbi10 promoter in Z. mays. Guide RNAs were designed and expressed from U6 promoters—either the A. thaliana U6 promoter for dicots or the O. sativa U6 promoter for monocots. A complete list of plasmids used in these studies is found in TABLE 1.

TABLE 1
gRNA constructs (sequences provided
in the attached Sequence Listing)

| Name | SEQ ID NO: |
|---|---|
| AtU6 LacO sgRNA | 1 |
| AtWUS 4x gRNA repeat | 2 |
| OsU6 LacO sgRNA | 3 |
| AtCLV3g1 | 4 |
| AtCLV3g2 | 5 |
| FT BlockB gRNA | 6 |
| FT BlockC gRNA | 7 |
| FT BlockE gRNA | 8 |
| FTgA gRNA | 9 |
| FTgB gRNA | 10 |
| FT IntB gRNA | 11 |
| FT IntC gRNA | 12 |
| FT IntE gRNA | 13 |
| Pap1 gRNA | 14 |
| WUS gRNA | 15 |

Protoplast isolation and transformation. S. viridis and A. thaliana protoplasts were isolated and transformed as described elsewhere (Yoo et al., Nature Protocols 2(7):1565-1572, 2007; and Sychla et al., In Protoplast Technology, Methods in Molecular Biology, Wang and Feng, eds., vol. 2464, pp. 223-244, Humana, New York, NY, 2022). ME034 S. viridis plants were grown in a growth chamber set to a 31° C. and 21° C. diurnal cycle. Mesophyll cells were isolated from leaf tissue 14-21 days post germination. Col-0 A. thaliana plants were grown in a growth chamber set to 22° C. with a 16 hour light cycle. Mesophyll cells were isolated from leaf tissue 14-21 days post germination. Zea mays protoplasts were isolated and transformed as disclosed elsewhere (Cao et al., Acta Physiologiae Plantarum 36:1271-1281, 2014). Z. mays plants were grown in a growth chamber at 25° C. under a 16 hour light cycle. Prior to isolation, plants were “greened” by first placing the seedlings in the dark for 5 days post-germination, followed by 2 days of exposure to light prior to isolation. Plasmids to be transformed in all systems were midi prepped according to the manufacturer's protocol (Qiagen 12945; Qiagen, Hilden, Germany) to ensure transformation grade endotoxin free DNA.

Dual luciferase assay. A dual luciferase assay was designed with the Firefly and Renilla luciferase reporter genes from Promega (Sherf et al., Promega Notes Magazine Number 57, page 2, 1996). A Renilla luciferase was cloned upstream of a Firefly luciferase on a single plasmid. The Renilla coding sequence (CDS) was under the control of a constitutive promoter, either the stronger CaMV 35S promoter or the weaker Nos promoter. Upstream of the Firefly CDS was a minimal CaMV 35S promoter containing only a transcription start site. Six copies of the Lac Operator were cloned upstream of the minimal promoter driving Firefly expression. An sgRNA targeting the LacO region resulted in targeting of dCas9 activation to the Firefly promoter. Expression of dCas9-24xGCN4 was driven by a constitutive promoter (CMYLCV, AtUbi10, or ZmUbi1), expression of the LacO sgRNA was driven by the O. sativa U6 promoter, and expression of the scFv was driven by a constitutive 35S promoter. S. viridis, A. thaliana, or Z. mays protoplasts were co-transformed with the dCas9-24xGCN4, LacO sgRNA, scFv-AD, and dual luciferase reporter plasmids. Following a 24 hour incubation at 25° C., the cells were lysed in 1× Passive Lysis Buffer from Promega. Firefly and Renilla luciferase luminescence was quantified using a GloMax Explorer plate reader (Promega GM3500; Promega, Madison, WI) equipped with dual injectors, and a Dual Luciferase Assay kit (Promega E1960). The Firefly luciferase substrate was injected first and luminescence was quantified, and then the Renilla luciferase substrate was injected and the resulting luminescence was quantified. Delivery was normalized by dividing Firefly luminescence by Renilla luminescence unless otherwise described, followed by Fold Change calculation by dividing a given AD by the negative control or VP64. A complete list of plasmids used in these studies is found in TABLE 2.

TABLE 2
Coding sequences (sequences provided
in the attached Sequence Listing)

| Name | SEQ ID NO: |
|---|---|
| AtUbi10-dCas9-24xGCN4 | 16 |
| CmYLCV-dCas9-24xGCN4 | 17 |
| p6xLacOfLuc | 18 |
| pABI5 | 19 |
| pAda2b | 20 |
| pAFT1 | 21 |
| pAL2-TrAP | 22 |
| pAP1 | 23 |
| pAsf1a | 24 |
| pAsf1b | 25 |
| pAtARF8 | 26 |
| pAtARR2 | 27 |
| pATHB13 | 28 |
| pAtHSFA2 | 29 |
| pAtHSFA6b | 30 |
| pATXR7 | 31 |
| pAvrXa10 | 32 |
| pBBX21 | 33 |
| pbHLH122 | 34 |
| pBRE1 | 35 |
| pCVBp12 | 36 |
| pDof1 | 37 |
| pDREB1 | 38 |
| pDREB2 | 39 |
| pDREB2-ZmUbi1 | 40 |
| pDualLuc-35S | 41 |
| pDualLuc-Nos | 42 |
| pEFS | 43 |
| pEIN3 | 44 |
| pGT3a | 45 |
| pHAG1 | 46 |
| pIPN2 | 47 |
| pLBD2 | 48 |
| pLMI1 | 49 |
| pMYC2 | 50 |
| pOpaque2 | 51 |
| pOsCBT | 52 |
| pOsGRF1 | 53 |
| pPTI4 | 54 |
| pRen-35S | 55 |
| pRF2a | 56 |
| pSDG31 | 57 |
| pSNAC1 | 58 |
| pTBP1 | 59 |
| pVRN1 | 60 |
| pWRKY50 | 61 |
| pZmUbi1-dCas9-24xGCN4 | 62 |
| pZmVP1 | 63 |

RNA isolation and quantification. RNA was isolated from either protoplasts or leaf tissue according the TRIzol manufacturer's protocol (Thermo 15596026; ThermoFisher Scientific, Waltham, MA). Protoplasts were spun down at 500 g for 2 minutes, followed by removal of W5 buffer before re-suspending cells in 1 mL of TRIzol. For plant tissue samples, the samples were snap frozen in liquid nitrogen, followed by shaking in a paint shaker apparatus with metal beads to homogenize the frozen tissue. 1 mL of TRIzol was added to the homogenized tissue, and the TRIzol protocol was followed according to the manufacturer's specifications. The samples were then treated with the Turbo DNA Free Kit (Invitrogen AM1907; ThermoFisher) to remove any plasmid and/or genomic DNA from the RNA sample. To quantify a transcript, RT-qPCR was performed using gene-specific primer pairs. A list of primers used for quantification is found in TABLE 3. The RT-qPCR cycling protocol was as defined by the NEB LUNA® One-Step RT-qPCR kit manufacturer's protocol (NEB E3005L; New England Biolabs, Ipswich, MA). Primers were designed to have Tm values of 55° C. and yield amplicon lengths between 75 and 175 bp. The primers typically spanned an intron, such that the shorter PCR product would correspond to spliced mRNA. Gene expression was quantified for relative comparison between treatments using the delta delta Ct method. All primers are listed TABLE 3.

Plant transformation and genotyping. To generate transgenic A. thaliana plants, floral dip protocols were performed as described elsewhere (Clough and Bent, The Plant Journal, 16(6):735-743, 1998). TDNA plasmids were constructed, notably containing an antibiotic resistance gene along with the pFAST Oleosin-RFP transgene. A. thaliana ecotype Columbia-0 was grown to maturity and floral dipped with Agrobacterium strain GV3101 transformed with the described TDNAs. The antibiotic selection used in these studies was either Kanamycin or Bialaphos resistance. TO seeds were identified by the pFAST system in which red fluorescent protein (RFP) indicated transgenic seedlings. RFP⁺ seedlings were placed directly onto soil or onto selective media to grow T1 plants. T1 plants were grown to maturity and allowed to set seed. All molecular characterization was performed on T2 lines, along with phenotyping. To quantify FT over-expression phenotypes in T2 plants, 18 RFP positive T2 plants from each T1 parent were planted. Time point 0 was set as the date on which the first set of true leaves emerged from a seedling. The number of days elapsed until bolting was observed were then counted. The number of rosette leaves also were counted one day after bolting was observed. RT-qPCR was performed to quantify target gene expression, using the primers listed in TABLE 3.

TABLE 3
qRT-PCR primers

| | | SEQ ID |
|---|---|---|
| Name | Sequence (5′→3′) | NO: |
| AtPP2a Fwd | AACGTGGCCAAAATGATGC | 64 |
| AtPP2a Rev | AACCGCTTGGTCGACTATCG | 65 |
| AtWUS Fwd | CAGCTTCAATAACGGGAATTT | 66 |
| AtWUS Rev | GTTGTAAGGTGCAGATGAGT | 67 |
| AtLec1 Fwd | CAGAGAAACAATGGAACGTG | 68 |
| AtLecl Rev | TGAGACGGTAAGGTTTTACG | 69 |
| AtClv3 Fwd | GTTCAAGGACTTTCCAACCGCAAGATGAT | 70 |
| AtClv3 Rev | CCTTCTCTGCTTCTCCATTTGCTCCAACC | 71 |
| AtFT Fwd | CCCTGCTACAACTGGAACAAC | 72 |
| AtFT Rev | CACCCTGGTGCATACACTG | 73 |
| AtPAP1 Fwd | AGTATGGAGAAGGCAAATGGC | 74 |
| AtPAP1 Rev | CACCTATTCCCTAGAAGCCTATG | 75 |

### Example 2—Building a Library of Putative Plant-Derived Transcription Activation Domains

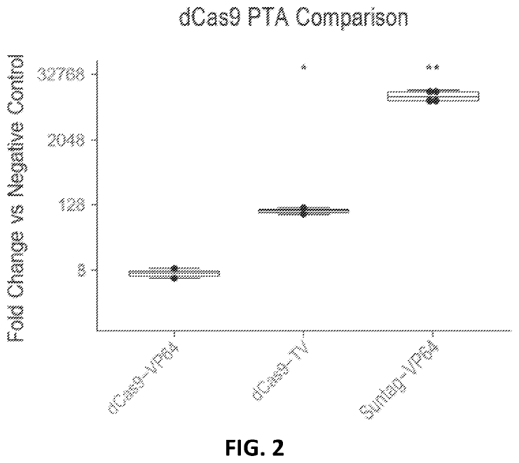

A system for identifying strong activation domains using protoplasts was developed. Because protoplast isolation and transformation pipelines yielded consistent cell number and transformation efficiencies (Yoo et al., supra; and Sychla et al., supra), it was possible to directly compare groups within a given transformation when activating a reporter gene. The ability of direct fusion PTA architectures (FIG. 1A, top) such as dCas9-VP64 to activate an endogenous target gene in A. thaliana was compared to the ability of AD-scaffolding PTA architectures like SunTag (FIG. 1A, bottom) to activate the target gene. A tRNA-based multi-guide expression array (Cermik et al., The Plant Cell, 29(6):1196-1217, 2017) expressing four sgRNAs targeting a single core promoter for Wuschel (WUS) was transformed into A. thaliana protoplasts along with a dCas9 PTA, followed by RNA extraction and RT-qPCR. The dCas9-PTAs were expressed using the A. thaliana Ubi10 promoter. One day post-transformation, all PTAs increased the expression of WUS compared to a no-sgRNA control. While dCas9-TV outperformed dCas9-VP64, the SunTag-VP64 design outperformed both direct-fusion PTAs by two orders of magnitude (FIG. 2). These results suggested that a library of putative ADs tested as fusions to scFv in the SunTag system would result in a dynamic range of activation for comparison.

To identify effective ADs for plants, transcription factors with known DNA binding domains were examined, and genes from diverse plant transcription factor families were selected. Native DNA-binding domains were computationally removed to reduce the likelihood of off target effects, and a putative activation domain motif was selected for each transcription factor. If an AD was not empirically determined, motifs enriched in acidic and/or aromatic residues were typically selected, as these patches can be associated with transcription activation domains due to their propensity to form phase separation condensates upon recruitment to a core promoter (Boija et al., Cell, 175(7):1842-1855, 2018). AD sequences from plant pathogen effector proteins also were added, including transcription-like effector proteins from Xanthomonas and sequences derived from transcription preinitiation complexes such as 14-3-3 scaffolding proteins. The name, sequence, and citation for each domain tested are listed in TABLE 4. Low amino acid sequence identity was observed across coding sequences from which the ADs were derived, as illustrated by the low-confidence boot-strap values in a Maximum Likelihood tree comparing these sequences (FIG. 1D). This highlighted the aim of the screen to survey a diverse array of plant-related ADs, versus optimization of a high-performing family.

Putative AD sequences were codon optimized based on average codon usage tables for A. thaliana and S. viridis for reliable expression across a variety of plant backgrounds, and were synthesized as dsDNA fragments. Type US restriction enzyme cloning was used to assemble putative AD coding sequences into an scFv destination vector. The ADs were expressed as C-terminal fusions to scFv, separated by a flexible GS linker. Many of the ADs were assembled and sequenced correctly, although certain ADs could not be assembled into the scFv vector. ADs that were not successfully assembly as a direct fusion to scFv were removed from the study.

TABLE 4
Activation domains (sequences provided in the attached Sequence
Listing; select sequences also provided herein)

| Name | Domain | AA range | DOI | UNIPROT No. | SEQ ID NO: |
|---|---|---|---|---|---|
| Dof1 | Plant | 175-238 | 10.1093/pcp/pce105 | P38564 | 76 |
| DREB1 | Plant | 108-216 | 10.1111/pbi.12057 | Q9M0L0 | 77 |
| DREB2 | Plant | 254-335 | 10.1111/pbi.12057 | O82132 | 78 |
| IPN2 | Plant | 179-358 | 10.1111/nph.12593 | E5L8F7 | 79 |
| PTI4 | Plant | 170-234 | 10.1111/pbi.12057 | O04680 | 80 |
| AtARF8 | Plant | 350-702 | 10.1105/tpc.008417 | Q9FGV1 | 81 |
| SNAC1 | Plant | 172-314 | 10.1073/pnas.0604882103 | A2XNB9 | 82 |
| Opaque-2 | Plant | 41-227 | PMC146487 | P12959 | 83 |
| AvrXa10 | Bacterial | | 10.1094/MPMI.1998.11.8.824 | Q56830 | 84 |
| AL2/C2/TrAP | Viral | 83-129 | 10.1006/viro.1999.9925 | P03562 | 85 |
| AtARR2 | Plant | 279-664 | 10.1111/j.1365-313X.2000.00909.x | Q9ZWJ9 | 86 |
| RF2a | Plant | 56-108 | 10.1074/jbc.M304862200 | Q69IL4 | 87 |
| RF2a | Plant | 283-357 | 10.1074/jbc.M304862200 | Q69IL4 | 88 |
| OsCBT | Plant | 542-755 | 10.1074/jbc.M504616200 | Q7XHR2 | 89 |
| AP1 | Plant | 193-256 | 10.1023/A:1006273127067 | P35631 | 90 |
| MYC2 | Plant | 148-188 | 10.1016/j.celrep.2017.04.057 | Q39204 | 91 |
| ZmVP1 | Plant | 1-120 | 10.1016/0092-86749190436-3 | P26307 | 92 |
| OsGRF1 | Plant | 221-396 | 10.1093/pcp/pch098 | A2XA73 | 93 |
| AtHSFA2 | Plant | 230-341 | 10.1111/j.1365-313X.2004.02111.x | O8098 | 94 |
| AtHSFA6b | Plant | 252-391 | 10.1111/j.1365-313X.2004.02111.x | Q9LUH8 | 95 |
| EFS | Plant | 820-1230 | 10.1105/tpc. 109.070060 | F4I6Z9 | 96 |
| AFT1 | Plant | Full length | 10.1016/S0014-57939801739-6 | P48349 | 97 |
| EIN3 | Plant | 310-620 | 10.1016/j.jmb.2005.02.065 | O24606 | 98 |
| WRKY50 | Plant | 1-106 | 10.3389/fpls.2018.00930 | Q8VWQ5 | 99 |
| ATXR7 | Plant | 1250-1423 | 10.1105/tpc.109.070060 | F4K1J4 | 100 |
| ASF1a | Plant | Full length | 10.1111/pce. 12299 | Q9C9M6 | 101 |
| HAG1 | Plant | 150-550 | 10.1093/nar/29.7.1524 | Q9AR19 | 102 |
| GT-3A | Plant | 110-323 | 10.1016/S0014-57930400222-4 | Q9SDW0 | 103 |
| CVBp12 | Virus | | 10.1105/tpc. 112.106476 | P37992 | 104 |
| ASF1b | Plant | Full length | 10.1111/pce.12299 | Q9LS09 | 105 |
| TBP1 | Plant | | na | P28147 | 106 |
| BRE1 | Plant | 700-876 | na | Q8RXD6 | 107 |
| Transcription | | | | | |
| factor | Gene ID | AA range | Source | UNIPROT No. | SEQ ID NO: |
| bHLH122 | At1g51140 | 1-300 | DAP seq dataset | Q9C690 | 108 |
| LMI1 | AT5G03790 | 140-234 | DAP seq dataset | Q9LZR0 | 109 |
| SDG31 | AT3G04380 | 196-462 | DAP seq dataset | Q8W595 | 110 |
| VRN1 | At3g18990 | 100-240 | DAP seq dataset | Q8L3W1 | 111 |
| ATHB13 | AT1G69780 | 151-294 | DAP seq dataset | Q8LC03 | 112 |
| ABI5 | AT2G36270 | 1-340 | DAP seq dataset | Q9SJN0 | 113 |
| LBD2 | AT1G06280 | | DAP seq dataset | Q9LNB9 | 114 |
| BBX21 | AT1G75540 | 110-331 | DAP seq dataset | Q9LQZ7 | 115 |

### Example 3—A Set of Plant-Evolved ADs Show Comparable Activity to VP64 in a Dual Luciferase Protoplast Assay

A reporter assay was designed to rapidly screen the library for domains capable of activating transcription in plant cells based on the dual luciferase assay (Sherf et al., supra; and Luehrsen et al., Meth. Enzymol., 216(C):397-414, 1992). A reporter construct was designed in which a synthetic promoter sequence containing six copies of the Lac operator upstream of the minimal 35S CaMV core promoter, which was upstream of Firefly luciferase (Benfey and Chua, Science, 250(4983):959-966, 1990). An sgRNA targeting the LacO sequence resulted in dCas9 binding to all six LacO motifs, such that firefly luciferase expression could be used as a reporter of AD strength. To provide an internal control for plasmid copy number, a sequence encoding Renilla luciferase controlled by a constitutive promoter was inserted into the same plasmid, upstream of and oriented in the same direction as the Firefly luciferase (FIG. 1E, top). Alternative ADs were expressed a scFv-AD fusions was driven by a 35S promoter, allowing for strong expression in both monocot and dicot protoplasts. The dCas9-24xGCN plasmid was expressed under control of the AtUbi10 promoter in A. thaliana or the CMYLCV promoter in S. viridis (FIG. 1B).

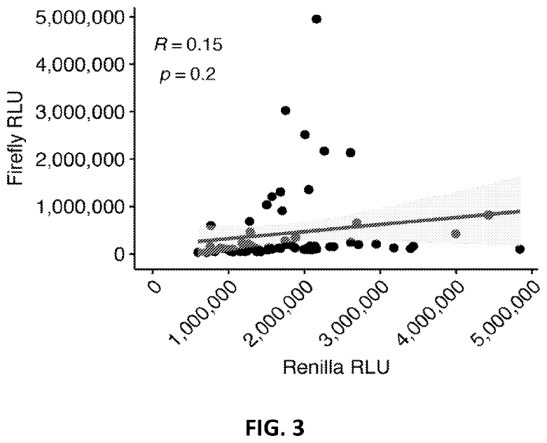

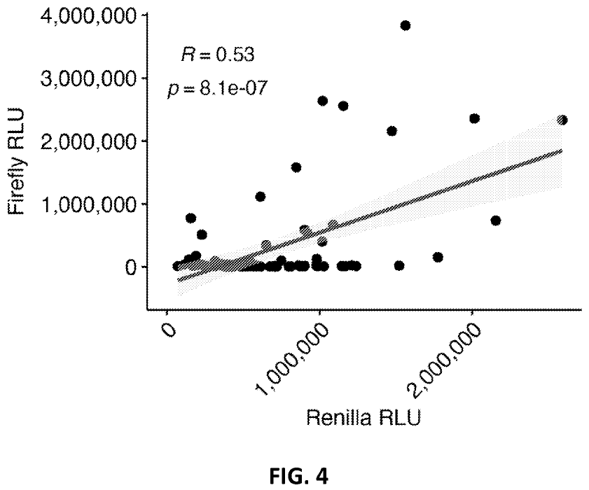

The library was first tested in S. viridis protoplasts. Cells (100,000) were isolated and transformed with 2 g of DNA in a 96 well plate format. Each transformation received dCas9-24xGCN, the LacO sgRNA, the single dual luciferase plasmid with 35S driving Renilla, and a given scFv-AD. Each scFv-AD was independently transformed twice per protoplast isolation. Following two protoplast isolations spanning four independent transformations, it was observed that DREB2, DREB1, and HSFA6b consistently produced the largest Renilla luciferase values across all four independent transformations. While the first dataset showed a weak correlation (R=0.15, FIG. 3), when fitting the second data set to a linear model, a strong correlation between Firefly and Renilla luciferase was noted (R=0.53, FIG. 4). This seemed unlikely, given the initial construct design in which Renilla luciferase values were expected to be a readout of transformation efficiency, but not to fit a model in which firefly luciferase expression was linearly correlated with Renilla luciferase expression. It was possible that the transcription activation machinery being recruited by the AD to the minimal 35S promoter may also have activated transcription at the nearby upstream Renilla luciferase promoter, and that strong ADs may have been activating nearby genes to a greater extent than weaker activation domains. If this was the case, driving expression of the Renilla luciferase with a weaker promoter would likely result in a better linear fit due to strong ADs at the Firefly luciferase minimal promoter activating expression of the nearby Renilla luciferase. To determine whether off target activation had been occurring, the library transformation studies were repeated with a weaker constitutive Nos promoter driving expression of the Renilla luciferase. Indeed, a strong linear correlation was observed between the Firefly and Renilla luciferase values when the upstream nearby Renilla luciferase was driven by the weak Nos promoter (FIG. 1C, top).

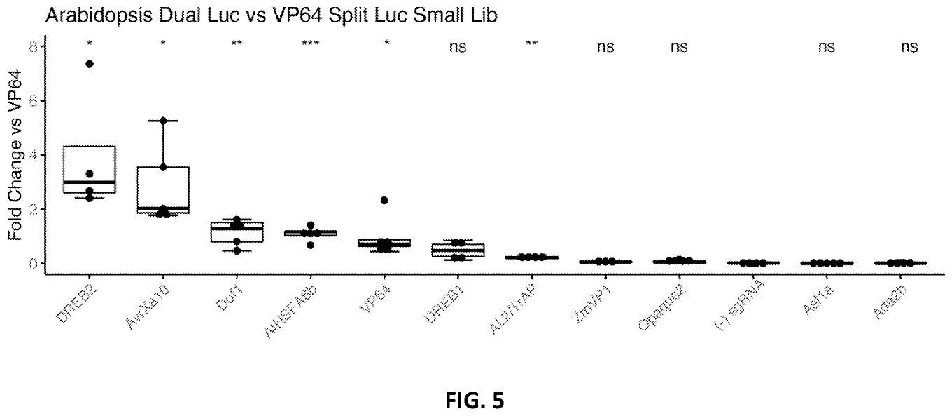

Given the results, it was decided to split the Firefly and Renilla luciferase coding sequences into separate plasmids (FIG. 1F). By placing the Firefly and Renilla luciferase promoters on separate plasmids, it was hypothesized that the likelihood of ADs at one promoter impacting the expression of another promoter on a separate plasmid would be low. The split luciferase platform was tested in A. thaliana protoplasts with a smaller library of ADs, revealing and a weak correlation for a linear regression model (FIG. 1F, bottom). RLU calculations were performed using Renilla as a copy number control, revealing that a similar set of core set of ADs was capable of activating transcription in A. thaliana for this experiment, albeit with variable strength compared to the Setaria datasets (FIG. 5). DREB2 retained high activity, and AvrXa10 performed better in A. thaliana than in S. viridis. Conversely, DREB1, HSFA6b, and ZmVP1 all displayed lower levels of activation in the A. thaliana dicot model.

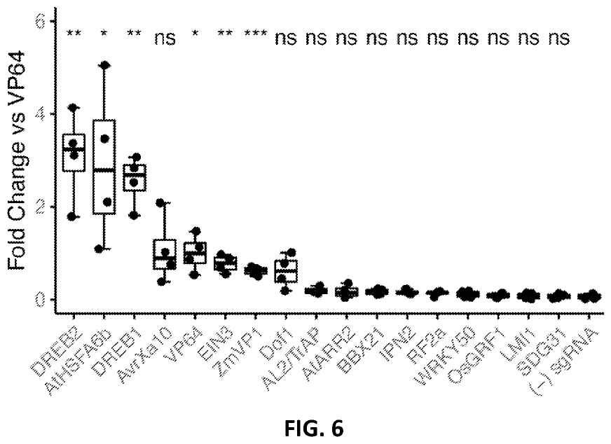

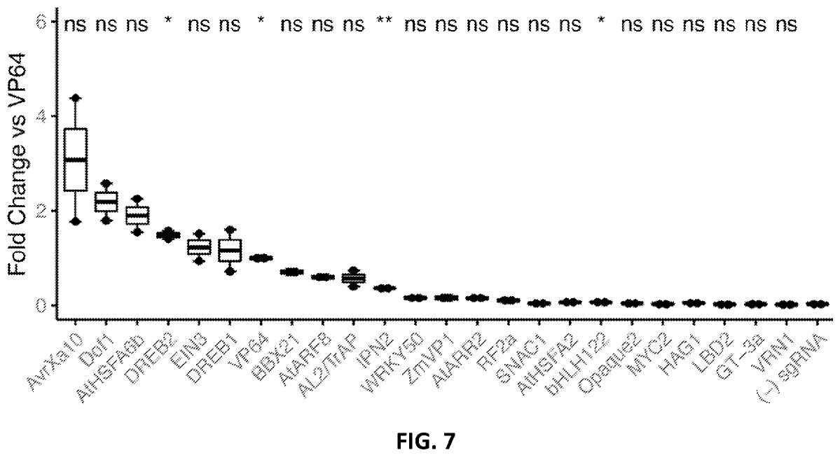

Given the suggestion of local off target activation at the Renilla luciferase, it was decided to display the Setaria data simply as raw Firefly values compared to the VP64 positive control. When performing this analysis, a core set of seven (7) activation domains (DREB2, HSFA6b, DREB1, AvrXa10, EIN3, ZmVP1, and Dof1) showed comparable or greater activity than the conventionally used VP64 activation domain (FIG. 1G). Notably, DREB2, HSFA6b, and DREB1 showed the largest Firefly activity across four independent transformations. The full library data sets for both A. thaliana and S. viridis are presented in FIGS. 6 and 7.

Finally, to determine how the plant-derived TADs performed in a crop species, another small library of strong plant ADs was tested in the model crop Z. mays using the split dual luciferase assay. Z. mays protoplasts were isolated from greened tissue, and cells were transformed with AtHSFA6b, AvrXa10, Dof1, DREB1, DREB2, and VP64 ADs in the SunTag system. Both the dCas9-24xGCN and scFv-AD constructs were driven by the ZmUbi1 promoter for strong expression. Luciferase expression analysis was performed using Renilla luciferase as the copy number control, revealing that both DREB2 and Dof1 retained strong activity in the model crop Z. mays, with 3-fold greater activity for DREB2 as compared to VP64 (FIG. 8). Interestingly, the AvrXa10 domain decreased in activity within Z. mays protoplasts relative to its strength in A. thaliana protoplasts. Taken together, these studies demonstrated that HSFA6b, AvrXa10, Dof1, DREB1, and DREB2 exhibited promising activity across all three species tested, when compared to the standard VP64 AD.

### Example 4—DREB2, Dof1, and AvrXa10 Plant-Derived Activation Domains Outperform VP64 Across Endogenous Loci

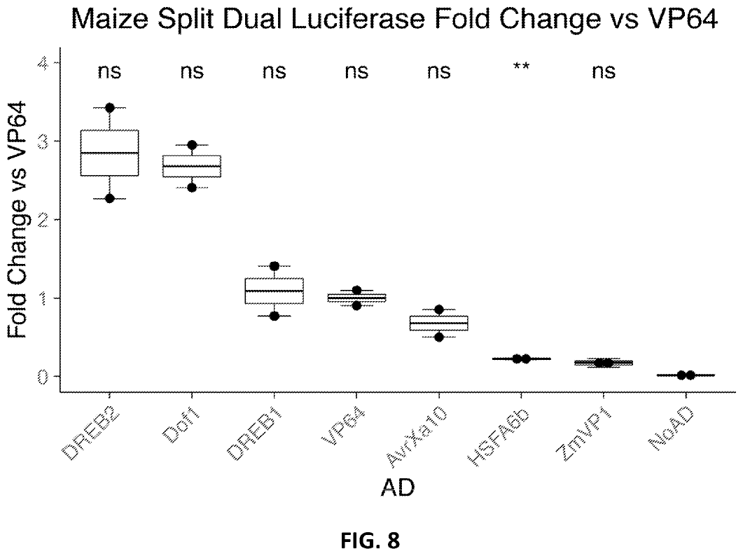

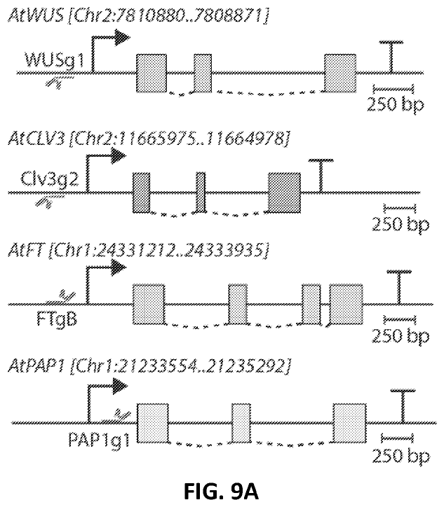

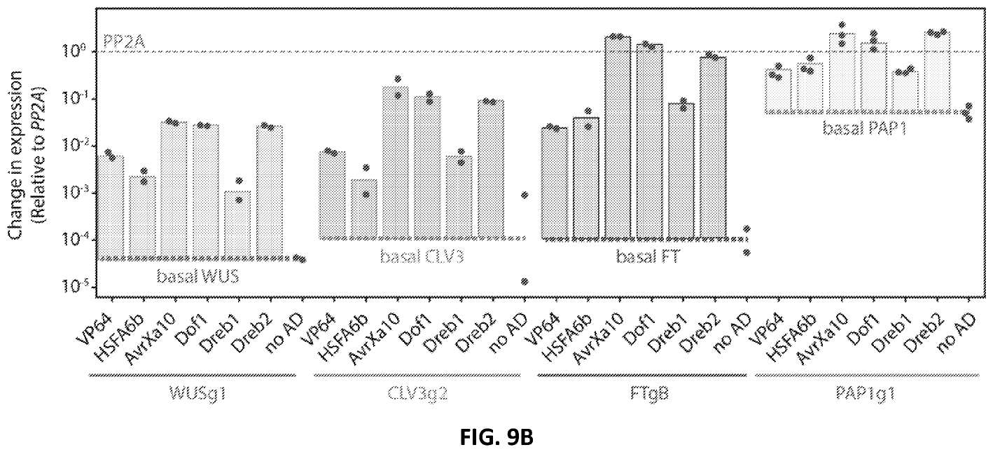

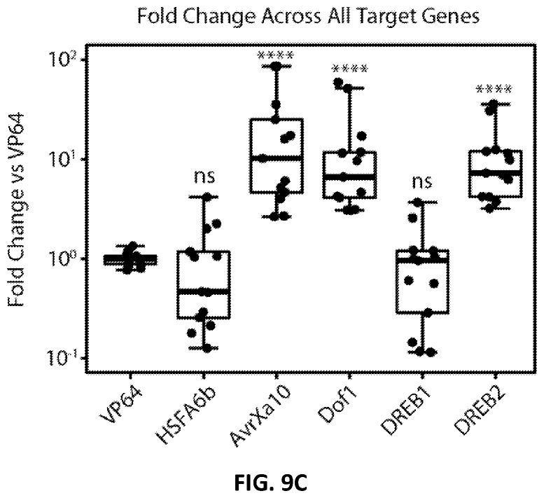

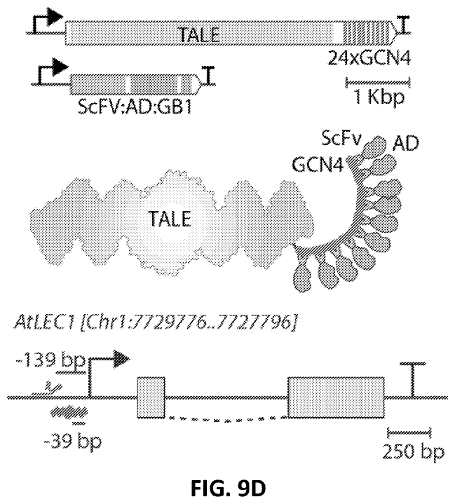

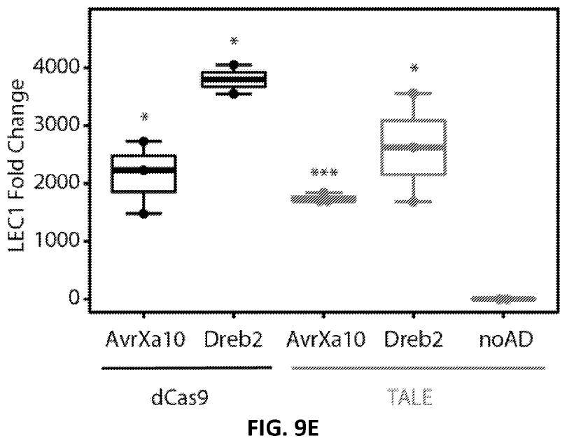

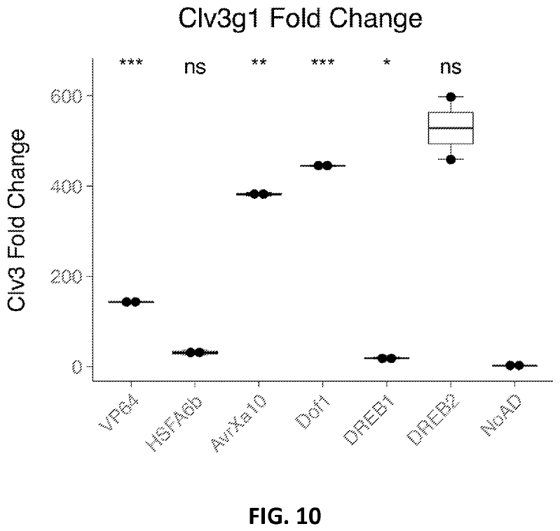

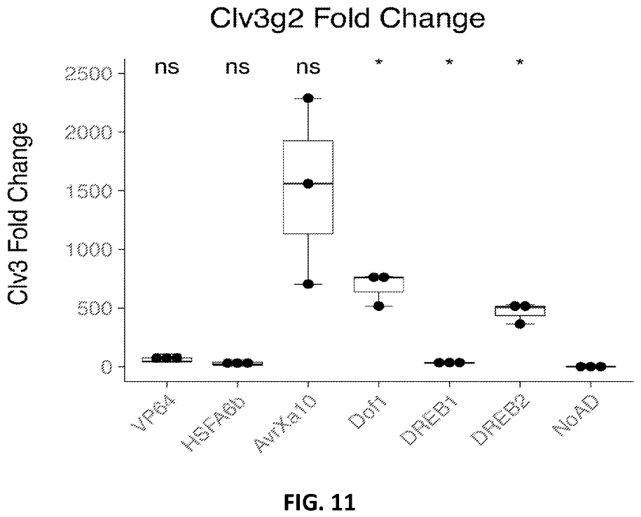

Studies were conducted to test the efficiency of HSFA6b, AvrXa10, Dof1, DREB1, DREB2, and VP64 at endogenous loci. Single guide RNAs expressed from the A. thaliana U6 promoter were constructed to target the core promoters of Flowering Locus T (FT), Clavata3 (Clv3), Production of Anthocyanin Pigment 1 (PAP1), and Wuschel (WUS) for activation (FIGS. 9A, 9B, and 10-12). All sgRNAs used in this study were described elsewhere (Lowder et al., supra; and Lee et al., PLOS ONE, 14(9):e0222778, 2019), except that the Wuschel guide was newly developed.

A. thaliana protoplasts were isolated and transformed with the SunTag activator and U6-sgRNA, with either two or three replicates per activation domain per target gene. After 24 hours, RNA was isolated and RT-qPCR was performed to quantify gene activation. For every sgRNA tested, the activity of each AD was compared to the activity of a no-AD control in which the dCas9 and sgRNA were transformed without an scFvAD. For each sample, the target gene and a control housekeeping gene (PPa2) were amplified. Fold change was calculated using the delta delta Ct method (Livak and Schmittgen, Methods, 25(4):402-408, 2001). Expression is reported relative to PPa2 in FIG. 9B for FT, PAP1, and WUS. By graphing all data points relative to the housekeeping gene, it was easier to observe both the fold-change in gene expression (height of the bar), and the absolute change in gene expression (location relative to the y-axis). This was important, as the fold-change in expression achieved by the PTAs was inversely correlated with the basal expression level. Genes already expressed at high levels cannot be overexpressed to the same relative degree as genes with a low basal expression level (Chiarella et al., supra). It was confirmed that all primer pairs used in this analysis had the same efficiency of amplification.

The sgRNAs were targeted to the core promoter regions, typically located 0 to 300 base pairs upstream of an annotated transcription start site (TSS) for the gene, although the Pap1 gRNA used in this study interestingly bound downstream of the annotated TSS. A clear pattern emerged across sgRNAs tested, in which AvrXa10, Dof1, and DREB2 consistently produced greater gene activation than VP64, HSFA6b, and DREB1 (FIGS. 7, 8, 9A-9C, and 10). The rank order of AvrXa10, Dof1, and DREB2 changed depending on the guide being tested. As expected, the maximum fold-overexpression was correlated with basal expression level. For example, FT resulted in a much higher fold-overexpression compared to no-AD control (>10,000) than Pap I compared to no-AD control (50), but this was due to FT starting at a much lower basal expression level. The maximum absolute expression attained was comparable for FT and Pap1. WUS, on the other hand, could only be overexpressed to 10% of the level of Ppa2. To view the data in aggregate, the gene activation dataset of each AD was compared to that of VP64. After setting VP64 activation values for each gene tested equal to 1, the datasets for all sgRNAs were combined across four target genes (n=13). The same trend was evident, in that AvrXa10, Dof1, and DREB2 produced statistically significant gene activation across all target genes and guide RNAs that were tested (FIG. 9C).

Studies were then conducted to test the portability of these activation domains between different programmable DNA binding domains. The 24xGCN4 epitope tail from the SunTag system was cloned as a C-terminal fusion to a TALE programmable DNA binding domain. A TALE DNA binding domain was engineered by assembling a series of 20 TALE repeats, each of which contained a unique Repeat Variable Diresidue (RVD) (Lowder et al., supra) to specify a particular nucleotide for binding, such that a 20 base pair genomic target sequence was specified by a combination of 20 RVDs. TALE repeats were assembled to target a 20 bp sequence that was 39 bp upstream of the transcription start site for the A. thaliana Lec1 gene. In addition, an sgRNA targeted to a 20 bp sequence 139 bp upstream of the transcription start site for same Lec1 target gene also was generated (FIG. 9D). The ability of the AvrXa10 and DREB2 ADs to activate the Lec1 target gene in conjunction with either dCas9 or TALE DNA binding domains in A. thaliana was then tested. As expected, the dCas9 SunTag system using AvrXa10 and DREB2 domains resulted in strong Lec1 gene expression, with about 2100- or 3800-fold greater activation, respectively, than the negative control (FIG. 9E). The TALE SunTag system using AvrXa10 and DREB2 domains also resulted in strong Lec1 gene expression that was, respectively, about 1700- and 2600-fold greater activation than the negative control. These results demonstrated the flexibility of the AvrXa10 and DREB2 ADs to drive strong expression of target genes using different programmable DNA binding domains.

### Example 5—AvrXa10 and DREB2 Activation Domains Promote Early Flowering in Transgenic Plants

Further studies were conducted to test whether the plant-derived ADs would produce a phenotype upon gene activation in stable transgenic plants. T-DNA vectors were generated to express the plant-derived ADs in in a PTA architecture called the MoonTag system. Like SunTag, the MoonTag system utilizes an epitope-nanobody interaction to recruit the AD to the dCas9 molecule (Boersma et al., Cell, 178(2):458-472.e19, 2019). Twenty-four (24) copies of the GP41p epitope were fused to dCas9, while AvrXa10, Dof1, and DREB2 were fused to the GP41 2H10 nanobody (TABLE 5).

TABLE 5
T-DNA backbone sequences (provided
in the attached Sequence Listing)

| Construct | SEQ ID NO: |
|---|---|
| MT-AvrXa10-FT | 116 |
| MT-Dof1-FT | 117 |
| MT-DREB2-FT | 118 |

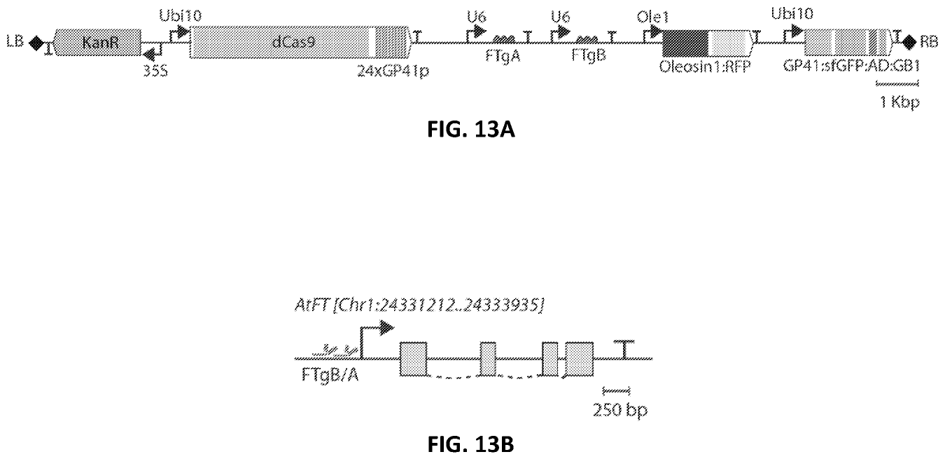

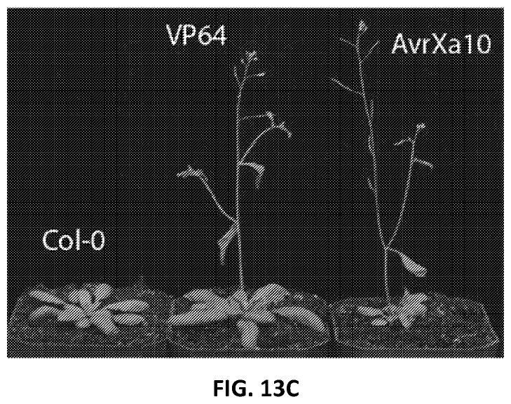

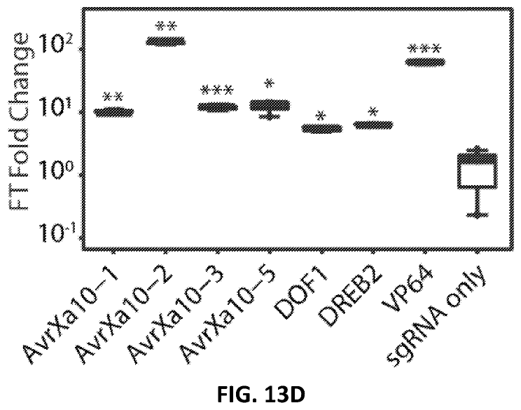

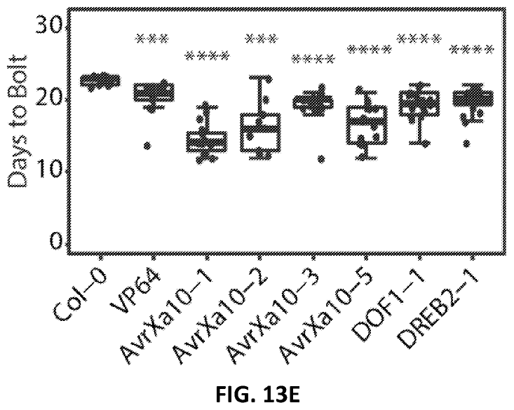

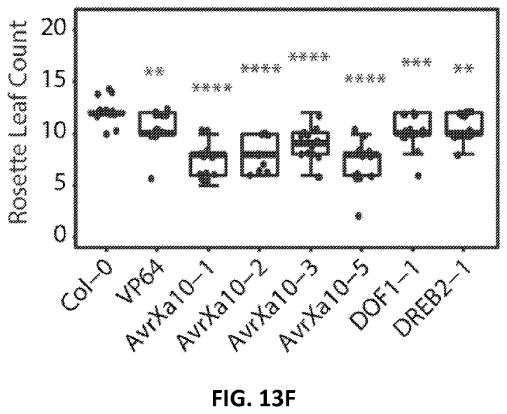

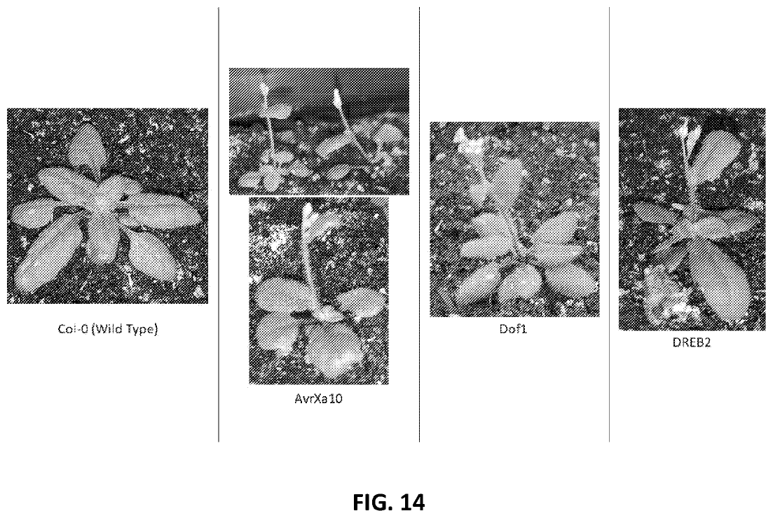

Two sgRNAs targeting the FT core promoter in A. thaliana were expressed from U6 promoters to drive FT gene activation (FIG. 13B). Plants were floral dipped and T1 transgenic lines were identified using the pFAST oleosin1 seed coat fluorescent reporter (Clough and Bent, The Plant Journal, 16(6):735-743, 1998; and Shimada et al., The Plant Journal, 61(3):519-528, 2010) (FIG. 13A). For comparison, a validated Moontag-VP64 transgenic line was used as the positive control, and wild type Columbia-0 (Col-0) plants were used as the negative control. In the T1 generation, many plants showed an early flowering phenotype (FIG. 14). These plants were allowed to set seed, and the homozygous plants in the T2 generation were used for quantification of flowering time. Sets of 20 seedlings per line were planted on selection media and allowed to germinate. Five days after germination, seedlings were pooled into groups of 6 and RNA was extracted from the pooled group. FT gene expression measured in the transgenic lines was compared to a control line expressing only sgRNAs against a different target gene. Strong gene activation was observed in the positive control VP64 line, along with the AvrXa10 transgenic lines. Activation was lower in both the Dof1 and DREB2 lines (FIG. 13C).

The FT overexpression phenotype in the T2 population was quantified by selecting 18 RFP-positive seedlings and planting them directly in soil. Following germination, the time point at which the seedlings produced their first set of true leaves was used as t=0. Two commonly used metrics of flowering time are bolting time and rosette leaf number (Takada and Goto, The Plant Cell, 15(12):2856-2865, 2003; and Krzymuski et al., The Plant Journal, 83(6):952-961, 2015). The day at which bolting was observed was noted, and the difference between this day and t=0 was calculated to quantify bolting time (FIG. 13D). The number of rosette leaves one day post bolting for each seedling was determined as an additional quantification of the FT phenotype (FIG. 13E). A strong FT activation phenotype was observed across multiple AvrXa10 transgenic lines, with early bolting and reduced rosette leaf number in comparison to wild type plants. Reduced rosette leaf size also was noted in several of the AvrXa10 plants (FIG. 13F, right panel) compared to wild type (left panels) and VP64 (center panels). In addition, early flowering phenotypes were observed for VP64, Dof1, and DREB2, albeit weaker than for the AvrXa10 lines. Taken together, these studies demonstrated that the plant-derived AvrXa10, Dof1, and DREB2 ADs functioned in transgenic plants, and that AvrXa10 showed the strongest molecular and phenotypic gene activation within the dicot species, A. thaliana.

### Example 6—PTA-Mediated Gene Enhancer Activation

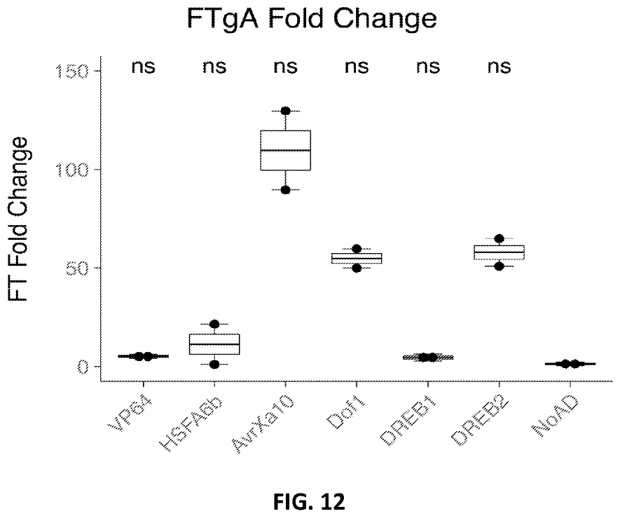

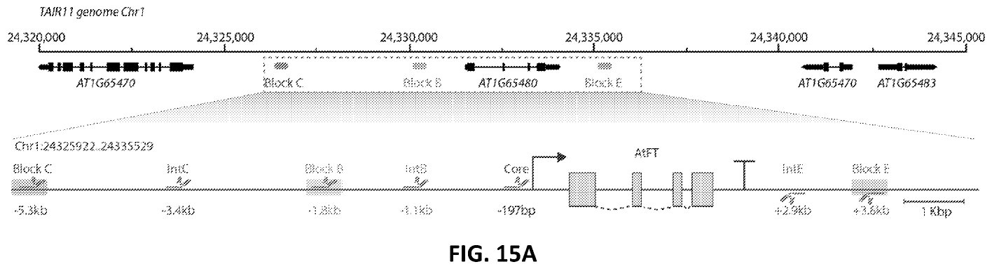

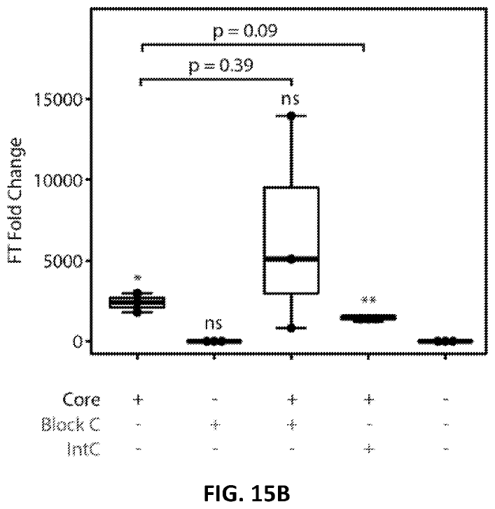

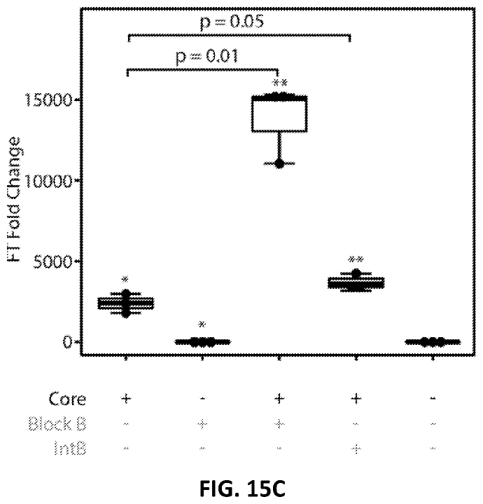

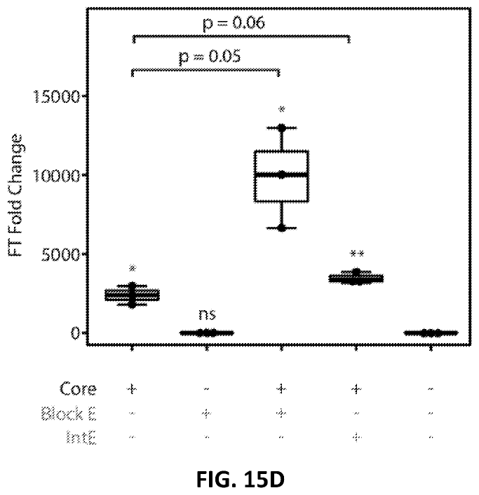

Work was then carried out to utilize the plant PTAs to map enhancers, long range cis elements, and the genes they regulate. The ability of the PTAs to activate enhancer regions was tested by co-targeting both a core promoter and enhancer element for gene activation (Tak et al., Nature Methods 18:9:1075-1081, 2021). The FT gene activation platform was utilized to illustrate this concept. Of the two sgRNAs tested, FTgB was capable of driving stronger gene activation than FTgA at the core promoter in the protoplast assays (FIGS. 9A and 12). As described elsewhere, Block B (−1.8 kb), Block C (−5.3 kb), and Block E (+3.8 kb) are three enhancers of FT located either upstream or downstream of the TSS (Johan Zicola et al., Nature Plants 5(3):300-307, 2019). An sgRNA targeting each FT enhancer was designed, along with the FT core promoter guide FTgB (FIG. 15A).

Targeting only the enhancer regions failed to produce observable gene activation, which was expected given the vast distances between the enhancers and the TSS. Indeed, there was no observable increase in FT expression above the negative control when targeting each individual enhancer alone (FIGS. 15B-15D). The FT core promoter was then targeted with a single guide, resulting in about 2400-fold activation of the FT target gene.

The FT core promoter and one of the three given enhancers were then co-targeted for activation. Co-targeting of the FT core promoter+Block C (−5.3 kb) resulted in variable activation that averaged to about a 6600-fold increase in FT expression relative to the negative control, a roughly 3-fold increase in expression relative to the FT core promoter alone (FIG. 15B). Co-targeting of the Core+Block B (−1.8 kb) resulted in the strongest observed activation, which was about 14,000-fold greater than the negative control, a roughly 6-fold increase relative to targeting the core promoter alone (FIG. 15C). Co-targeting of the FT core promoter+Block E (+3.8 kb) resulted in a 10,000-fold increase in FT expression relative to the negative control, a roughly 4-fold increase in expression relative to the core promoter alone (FIG. 15D). High variability was observed across the three independent transformations for Core+Block C, with the greatest activation being on par with Core+Block B (14,000-fold activation) and the lowest activation being on par with the Core promoter alone (1000-fold activation). While the Block C results were more ambiguous, the Block B and Block E results demonstrated that co-targeting of a core promoter+distal enhancer in A. thaliana significantly boosted target gene expression relative to targeting the core promoter alone.

### Example 7—Specificity of Plant-Derived ADs for sgRNA-Defined Target Sites

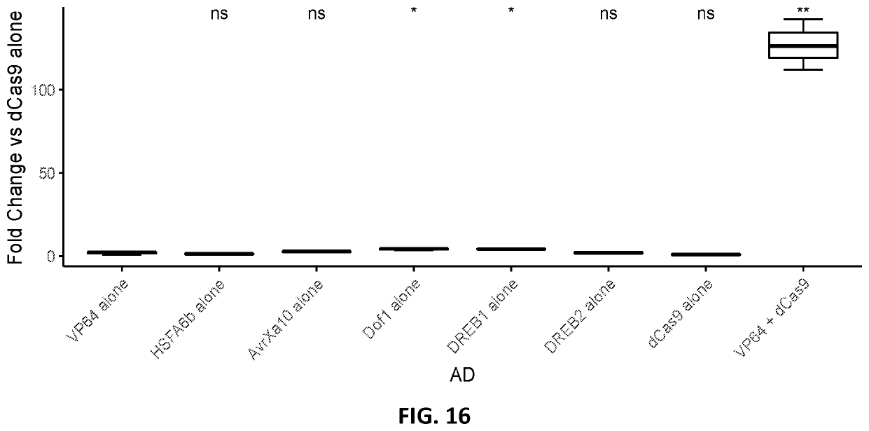

Because the ADs described herein originated plant backgrounds, it is possible that transcriptional networks unrelated to the CRISPR-targeted promoter could be activated. To confirm the specific quantification of each AD using the dual luciferase assay, basal luciferase expression was measured for each AD in the absence of dCas9. Any change in luciferase activity in the absence of dCas9 could be explained by a global non-specific increase in transcriptional activity due to constitutive expression of a given AD. Each scFv-AD was expressed in S. viridis protoplasts without the dCas9-24xGCN4 module. When compared to a negative control where no sgRNA was delivered, there was little to no observed increase in luciferase activity for each of the plant-derived AD treatments (FIG. 16). The only group showing activation of the Firefly luciferase reporter was the positive control, in which dCas9-24xGCN4 and scFv-VP64 were expressed with the sgRNA targeting the Firefly luciferase promoter. These results demonstrated that activation of the Firefly luciferase reporter requires both a strong plant-derived AD and the dCas9-24xGCN4+sgRNA complex.

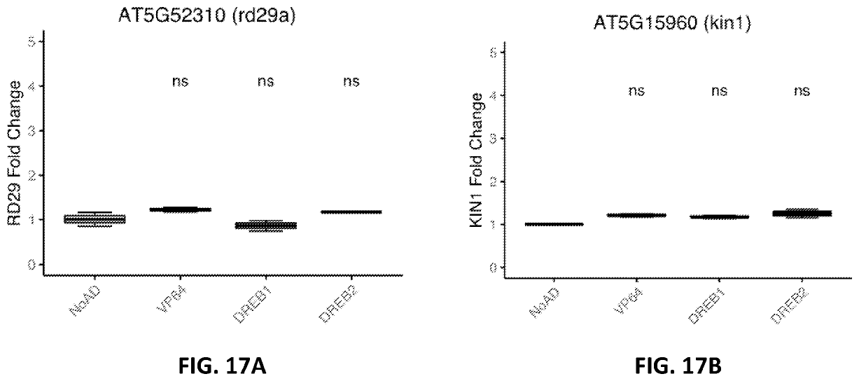

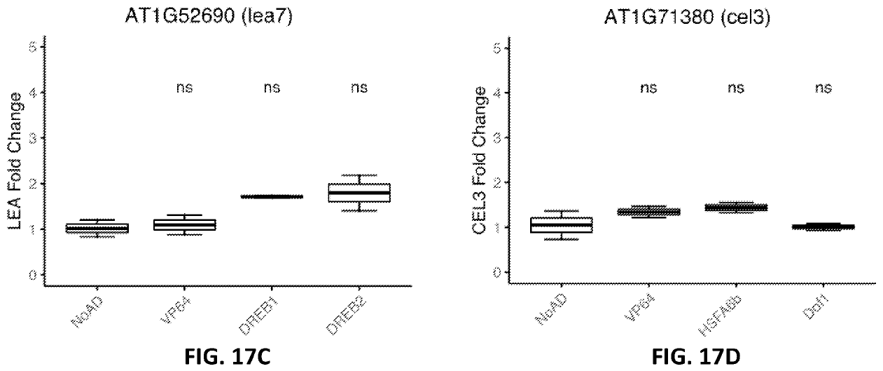

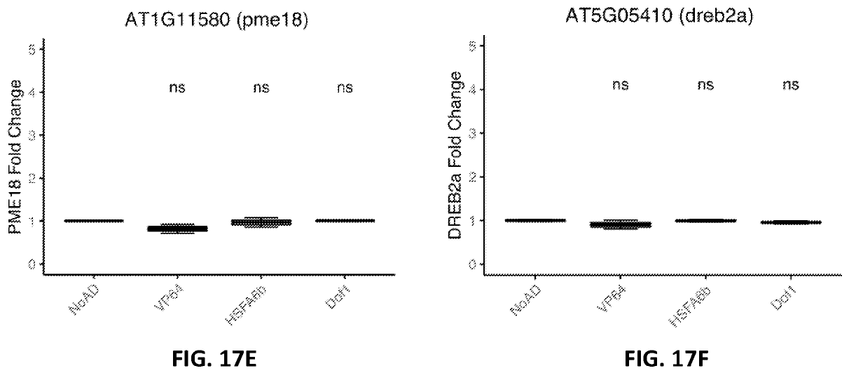

In addition to confirming the specificity of the Firefly luciferase reporter, the transcriptional change of downstream target genes for each plant-derived AD were quantified in A. thaliana protoplasts after treatment with the dCas9 activation complex targeting the FT locus, and unrelated target gene. These experiments were performed in A. thaliana protoplasts because the HSFA6b, DREB1, and DREB2 AD sequences were taken from endogenous Arabidopsis genes. Studies were therefore conducted to determine whether these ADs were still capable of activating downstream targets independent of the sgRNA-defined locus. Downstream target genes for HSFA6b, DREB1, DREB2, and TMO6 (the closest Dof1 homolog in A. thaliana) were identified (TABLE 6), and primers (TABLE 7) were designed to quantify the expression of the downstream target genes with RT-qPCR. The expression of a downstream target gene for the negative control (no AD) was compared to each AD treatment. Little to no off-target activation was detected for HSFA6b, DREB1, DREB2, or Dof1 (FIGS. 17A-17F). These results suggest specificity of the plant-derived ADs for the sgRNA-defined target site when fused to dCas9 in the scaffolding SunTag system.

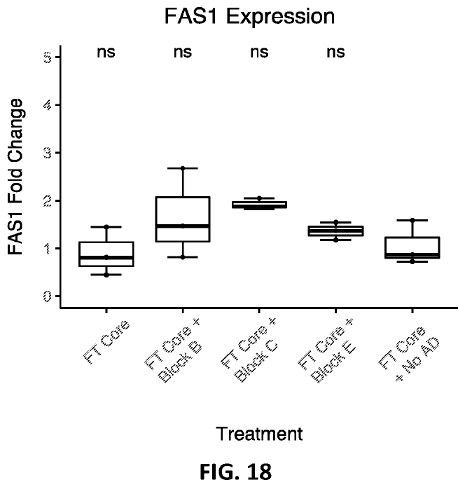

Finally, the specificity of PTA-mediated enhancer activation was tested by quantifying expression of the FAS1 gene located upstream of the FT gene. It is possible that the Block B, Block C, and Block E regions of open chromatin serving as enhancers for the FT locus may also act as enhancers for nearby genes when targeted with dCas9-based activators. To test this, RT-qPCR primers (TABLE 7) were designed for the FAS1 (AT1G65470) transcript, and its expression was measured across all enhancer treatments. The enhancer treatments surveyed for FAS1 expression in the study were targeting of the FT core promoter alone, co-targeting of the FT core promoter+Block B, co-targeting of the FT core promoter+Block C, co-targeting of the FT core promoter+Block E, and targeting the FT core promoter sgRNA+dCas9-24xGCN4 lacking any AD. Little activation was observed at the FAS1 gene when targeting the FT core promoter along with guides targeting the open chromatin enhancer regions of Block B, C, and E when compared to the No AD negative control (FIG. 18).

TABLE 6
Downstream target genes for measuring off-target activity

| AD | A. thaliana target 1 | A. thaliana target 2 |
|---|---|---|
| AtDREBla | rd29a (AT5G52310) | kin1 (AT5G15960) |
| AtDREB2a | rd29a (AT5G52310) | lea7 (AT1G52690) |
| ZmDof1 (AtTMO6) | cel3 (AT1G71380) | pme18 (AT1G11580) |
| AtHSFA6b | Dreba (AT5G05410) | rd29a (AT5G52310), |
| | | kin1 (AT5G15960) |

TABLE 7
qRT-PCR primers

| Name | Sequence (5′→3′) | SEQ ID NO: |
|---|---|---|
| rd29a Fwd | GTTGGAGGAAGAGTCGGCTG | 132 |
| rd29a Rev | TGCTTCTCGTCGACAAGTCTC | 133 |
| kin1 Fwd | ACAAGAATGCCTTCCAAGCC | 134 |
| kin1 Rev | CGGTCTTGTCCTTCACGAAG | 135 |
| lea7 Fwd | TGGTGAAACCAGAGGCAAGG | 136 |
| lea7 Rev | CGTTTGTGCAGCTTGAGACG | 137 |
| dreb2a Fwd | CAAGAAGACTAAACACGAAAGCG | 138 |
| dreb2a Rev | ACAGTAGTACCGTCACCTCTAC | 139 |
| cel3 Fwd | CATGGCTGCTAAGAGCAACG | 140 |
| cel3 Rev | AGATGAAATTCTCAGCTGCTTGC | 141 |
| pme18 Fwd | GCCTGCCAGAGACGATCTC | 142 |
| pme18 Rev | GTATTGCTGTTCTCCGGTGC | 143 |
| clv3 Fwd | TTCAAGGACTTTCCAACCGC | 144 |
| clv3 Rev | CTTGGTGGGTTCACATGATGG | 145 |
| fas1 Fwd | ATCTGAAAGGCTTTCACCAGC | 146 |
| fas1 Rev | AAGAAGTCCAGGAGGGTCAAG | 147 |
| pp2a Fwd | AACGTGGCCAAAATGATGC | 148 |
| pp2a Rev | AACCGCTTGGTCGACTATCG | 149 |

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide comprising a transcription activation domain (AD) and a DNA-binding domain, wherein the AD and the DNA-binding domain are not naturally present within the same protein, and wherein the AD comprises an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98.

2-15. (canceled)

16. The polypeptide of claim 1, wherein the DNA-binding domain comprises a dCas polypeptide, a transcription activator-like effector (TALE) polypeptide or a zinc finger binding domain.

17-18. (canceled)

19. A transcriptional activator system, comprising:
a first fusion polypeptide comprising (a) an AD portion and (b) a single-chain fragment variable (scFv) portion or a nanobody portion, wherein the AD portion comprises an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and
a second fusion polypeptide comprising (a) a DNA-binding domain and (b) one or more scFv or nanobody binding sequences.

20-33. (canceled)

34. The transcriptional activator system of claim 19, wherein the first fusion polypeptide comprises a scFv portion and the second fusion polypeptide comprises one or more scFv binding sequences, or wherein the first fusion polypeptide comprises a nanobody portion and the second fusion polypeptide comprises a nanobody binding sequence.

35-37. (canceled)

38. The transcriptional activator system of claim 19, wherein the second fusion polypeptide comprises ten or more scFv or nanobody binding sequences.

39. The transcriptional activator system of claim 19, wherein the DNA-binding domain comprises a dCas polypeptide, and wherein the transcriptional activator system further comprising a single guide RNA (sgRNA).

40-41. (canceled)

42. The transcriptional activator system of claim 19, wherein the DNA-binding domain comprises a TALE polypeptide or a zinc finger binding domain.

43. The transcriptional activator system of claim 19, wherein the first fusion polypeptide further comprises a solubility tag.

44. (canceled)

45. A nucleic acid comprising a nucleotide sequence encoding the polypeptide of claim 1.

46-59. (canceled)

60. The nucleic acid of claim 45, wherein the DNA-binding domain comprises a dCas polypeptide, a TALE polypeptide, or a zinc finger binding domain.

61-62. (canceled)

63. A transcriptional activator system, comprising:
a nucleic acid encoding a first fusion polypeptide that comprises (a) an AD portion and (b) a scFv portion or a nanobody portion, wherein the AD portion comprises an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and
a nucleic acid encoding a second fusion polypeptide comprising (a) a DNA-binding domain and (b) one or more scFv or nanobody binding sequences.

64-77. (canceled)

78. The transcriptional activator system of claim 63, wherein the first fusion polypeptide comprises a scFv portion and the second fusion polypeptide comprises one or more scFv binding sequences, or wherein the first fusion polypeptide comprises a nanobody portion and the second fusion polypeptide comprises a nanobody binding sequence.

79-81. (canceled)

82. The transcriptional activator system of claim 63, wherein the second fusion polypeptide comprises ten or more scFv or nanobody binding sequences.

83. The transcriptional activator system of claim 63, wherein the DNA-binding domain comprises a dCas polypeptide, and wherein the transcriptional activator system further comprises a nucleic acid encoding a sgRNA.

84-85. (canceled)

86. The transcriptional activator system of claim 63, wherein the DNA-binding domain comprises a TALE polypeptide or a zinc finger binding domain.

87. The transcriptional activator system of claim 63, wherein the first fusion polypeptide further comprises a solubility tag.

88. (canceled)

89. A method for activating transcription of a target gene in a plant cell, wherein the method comprises:
introducing a nucleic acid molecule into a plant cell, wherein the nucleic acid molecule comprises a nucleotide sequence encoding the fusion polypeptide of claim 1; and
allowing the cell to express the fusion polypeptide, such that the fusion polypeptide activates transcription of the gene.

90-103. (canceled)

104. The method of claim 89,
wherein the DNA-binding domain comprises a dCas polypeptide, and wherein the method further comprises introducing into the plant cell a nucleic acid encoding a single guide RNA (sgRNA); or
wherein the DNA-binding domain comprises a TALE polypeptide or a zinc finger binding domain.

105-106. (canceled)

107. The method of claim 89, wherein the DNA-binding domain is targeted to a promoter sequence of the gene.

108. The method of claim 107, wherein the method further comprises introducing a second nucleic acid molecule into the plant cell, wherein the second nucleic acid molecule comprises a nucleotide sequence encoding a second fusion polypeptide that comprises a second AD and a second DNA-binding domain targeted to the gene, wherein the second AD and the second DNA-binding domain are not naturally present within the same protein, wherein the second AD comprises an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98, and wherein the second DNA-binding domain is targeted to a potential or previously identified enhancer sequence of the gene.

109. A method for activating transcription of a target gene in a plant cell, wherein the method comprises:
introducing the transcriptional activator system of claim 63 into a plant cell; and
allowing the cell to express the first and second fusion polypeptides, such that transcription of the gene is activated.

110-123. (canceled)

124. The method of claim 109,
wherein the DNA-binding domain comprises a dCas polypeptide, and wherein the method further comprises introducing into the plant cell a nucleic acid encoding a sgRNA, or
wherein the DNA-binding domain comprises a TALE polypeptide or a zinc finger binding domain.

125-126. (canceled)

127. The method of claim 109, wherein the first fusion polypeptide comprises a scFv portion and the second fusion polypeptide comprises one or more scFv binding sequences, or wherein the first fusion polypeptide comprises a nanobody portion and the second fusion polypeptide comprises a nanobody binding sequence.

128-130. (canceled)

131. The method of claim 109, wherein the second fusion polypeptide comprises ten or more scFv or nanobody binding sequences.

132. The method of claim 109, wherein the first fusion polypeptide further comprises a solubility tag.

133. (canceled)

134. The method of claim 109, wherein the DNA-binding domain is targeted to a promoter sequence of the gene.

135. The method of claim 134, wherein the transcriptional activator system further comprises:
a nucleic acid encoding a third fusion polypeptide that comprises (a) a second AD portion and (b) a scFv portion or a nanobody portion, wherein the second AD portion comprises an amino acid sequence with at least 95% sequence identity to SEQ ID NO:78, SEQ ID NO:95, SEQ ID NO:84, SEQ ID NO:76, SEQ ID NO:77, SEQ ID NO:92, or SEQ ID NO:98; and
a nucleic acid encoding a fourth fusion polypeptide comprising (a) a second DNA-binding domain and (b) one or more scFv or nanobody binding sequences, wherein the second DNA-binding domain is targeted to a potential or previously identified enhancer sequence of the gene.


SEQUENCE LISTING (149 sequences)

SEQ ID NO: 1 - DNA, 2571 bp, synthetic construct
  source 1..2571: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgctagaaagaagaaagttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 2 - DNA, 3554 bp, synthetic construct
  source 1..3554: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcgggagagcgcccacctgccaggggacggcgcgcatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggaacaaagcaccagtggtctagtggtagaatagtaccctgccacggtacagacccgggttcgattcccggctggtgcataaatggggacccaaaaagagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaccctattgaaaaatgtgctgttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaaataaattttgccaaagaaagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcaataaaatgttatgtttaggagttttagagctagaaatagcaagttaaaataaggctagtccgttatcaacttgaaaaagtggcaccgagtcggtgcaacaaagcaccagtggtctagtggtagaatagtaccctgccacggtacagacccgggttcgattcccggctggtgcagtcgatcgacaagctcgagtttctccataataatgtgtgagtagttcccagataagggaattagggttcctatagggtttcgctcatgtgttgagcatataagaaacccttagtatgtatttgtatttgtaaaatacttctatcaataaaatttctaattcctaaaaccaaaatccagtactaaaatccagatcccccgaattagagctctaccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 3 - DNA, 2372 bp, synthetic construct
  source 1..2372: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatgggcgggatcatgaaccaacggcctggctgtatttggtggttgtgtagggagatggggagaagaaaagcccgattctcttcgctgtgatgggctggatgcatgcgggggagcgggaggcccaagtacgtgcacggtgagcggcccacagggcgagtgtgagcgcgagaggcgggaggaacagtttagtaccacattgcccagctaactcgaacgcgaccaacttataaacccgcgcgctgtcgcttgtgctagaaagaagaaagttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 4 - DNA, 2577 bp, synthetic construct
  source 1..2577: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgatttatagcgtaagcctacagttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 5 - DNA, 2577 bp, synthetic construct
  source 1..2577: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtgtgtagtaagtatatataggttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 6 - DNA, 2587 bp, synthetic construct
  source 1..2587: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgaaaatgatatacacaagtgggtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 7 - DNA, 2586 bp, synthetic construct
  source 1..2586: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgatagtgaagtgagacatctgtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 8 - DNA, 2586 bp, synthetic construct
  source 1..2586: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgcgaatgatcaagtcccaaggtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 9 - DNA, 2576 bp, synthetic construct
  source 1..2576: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggatttgcattaactcgggtgttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 10 - DNA, 2576 bp, synthetic construct
  source 1..2576: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtccagtgtattagtgtggtgttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 11 - DNA, 2587 bp, synthetic construct
  source 1..2587: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgatcctggtcgtgcaaatggagtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 12 - DNA, 2586 bp, synthetic construct
  source 1..2586: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgatgacaaagggcactcatggtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 13 - DNA, 2587 bp, synthetic construct
  source 1..2587: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgttgacagcataacgtctagagtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 14 - DNA, 2576 bp, synthetic construct
  source 1..2576: mol_type = other DNA
gccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgacagctaatacataaaatggttttagagctagaaatagcaagttaaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaat

SEQ ID NO: 15 - DNA, 2587 bp, synthetic construct
  source 1..2587: mol_type = other DNA
gcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgccaggggacccatggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtaaatggggacccaaaaagagtttaagagctatgctggaaacagcatagcaagtttaaataaggctagtccgttatcaacttgaaaaagtggcaccgagtcggtgctttttttcccggtgacgcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagta

SEQ ID NO: 16 - DNA, 9590 bp, synthetic construct
  source 1..9590: mol_type = other DNA
gggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgctcatgatcggcgcgccgtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggcctgcaggtagatcgctcgtcgacatggataagaagtactctatcggactcgctatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatgctatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggttggcggaccgggtggatctggaggtggaggttctggaggagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaagagttgctatcgaaaaattatcatcttgagaacgaagttgctaggctcaaaaagggctcaggctcaggcgaggagttgctctcgaaaaactaccacttggaaaatgaggtcgcgaggttgaaaaaggggagcgggtcgggcgaggagttattgagcaaaaactatcatttagagaacgaagtcgcgcgcttaaagaaaggctcgggctcgggcgaagaactcttatcgaagaactaccacctcgaaaatgaggtcgccaggttgaaaaagggcagtggcagcggggaggaactcttgagcaagaactaccacttggagaatgaggtcgcgagattgaagaaagggtcggggagcggcgaggaattgctcagcaagaattatcatttggagaacgaagtcgccaggctcaagaaaggctcggggtcgggggaggaattgttgagtaaaaactaccacttggaaaatgaagtcgccaggctcaaaaaagggagtgggagcggcgaagagttattgagcaaaaattaccacttggagaacgaagtggcaaggctcaagaaagggagcggcagcggggaggagctcttatcgaagaactaccacttagagaatgaagtcgcccgcttgaagaaaggctcggggagcggggaagagctcttgagcaagaactaccacttggaaaatgaggtggcgcgcttgaagaaagggagcgggagcggggaagagttactatctaagaattatcatctcgagaacgaggtggctcgactaaagaagggctccggcagtggggaggaactcctgtcgaagaactatcatcttgaaaatgaggttgcaagacttaaaaaggggtccggatcaggtgaggaactactcagtaagaattaccacctggaaaacgaagttgcacgtttgaagaaaggatcaggatcaggcgaagaactgctctcaaaagattatcatttggaaaatgaggttgcacgtttaaaaaagggaagtggcagtggtgaggaacttctgtcgaaaaattatcatctcgagaatgaagtagcccgacttaaaaagggttctggctcgggtggaggcccaaagaagaagaggaaagtctgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacgtgggcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggg

SEQ ID NO: 17 - DNA, 8728 bp, synthetic construct
  source 1..8728: mol_type = other DNA
gggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgctcatgatcggcgcgcctggcagacatactgtcccacaaatgaagatggaatctgtaaaagaaaacgcgtgaaataatgcgtctgacaaaggttaggtcggctgcctttaatcaataccaaagtggtccctaccacgatggaaaaactgtgcagtcggtttggctttttctgacgaacaaataagattcgtggccgacaggtgggggtccaccatgtgaaggcatcttcagactccaataatggagcaatgacgtaagggcttacgaaataagtaagggtagtttgggaaatgtccactcacccgtcagtctataaatacttagcccctccctcattgttaagggagcaaaatctcagagagatagtcctagagagagaaagagagcaagtagcctagaagtagtcaaggcggcgaagtattcaggcacgtggccaggaagaagaaaagccaagacgacgaaaacaggtaagagctaagcttcctgcaggtagatcgctcgtcgacatggataagaagtactctatcggactcgctatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatgctatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggttggcggaccgggtggatctggaggtggaggttctggaggagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaagagttgctatcgaaaaattatcatcttgagaacgaagttgctaggctcaaaaagggctcaggctcaggcgaggagttgctctcgaaaaactaccacttggaaaatgaggtcgcgaggttgaaaaaggggagcgggtcgggcgaggagttattgagcaaaaactatcatttagagaacgaagtcgcgcgcttaaagaaaggctcgggctcgggcgaagaactcttatcgaagaactaccacctcgaaaatgaggtcgccaggttgaaaaagggcagtggcagcggggaggaactcttgagcaagaactaccacttggagaatgaggtcgcgagattgaagaaagggtcggggagcggcgaggaattgctcagcaagaattatcatttggagaacgaagtcgccaggctcaagaaaggctcggggtcgggggaggaattgttgagtaaaaactaccacttggaaaatgaagtcgccaggctcaaaaaagggagtgggagcggcgaagagttattgagcaaaaattaccacttggagaacgaagtggcaaggctcaagaaagggagcggcagcggggaggagctcttatcgaagaactaccacttagagaatgaagtcgcccgcttgaagaaaggctcggggagcggggaagagctcttgagcaagaactaccacttggaaaatgaggtggcgcgcttgaagaaagggagcgggagcggggaagagttactatctaagaattatcatctcgagaacgaggtggctcgactaaagaagggctccggcagtggggaggaactcctgtcgaagaactatcatcttgaaaatgaggttgcaagacttaaaaaggggtccggatcaggtgaggaactactcagtaagaattaccacctggaaaacgaagttgcacgtttgaagaaaggatcaggatcaggcgaagaactgctctcaaaagattatcatttggaaaatgaggttgcacgtttaaaaaagggaagtggcagtggtgaggaacttctgtcgaaaaattatcatctcgagaatgaagtagcccgacttaaaaagggttctggctcgggtggaggcccaaagaagaagaggaaagtctgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacgtgggcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggg

SEQ ID NO: 18 - DNA, 4497 bp, synthetic construct
  source 1..4497: mol_type = other DNA
aaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgccggggcgcattgaggctctctacggtcacaggttgtgagccgttgttaaagacaaacaaatgctcgtgacttgcgaagatcgatccactagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagctccaccgcggtggcggccggccgtctagtggatcgattccccttcgcaagacccttcctctatataaggaagttcatttcatttggagaggacctgcaggatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgctggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgactcgaggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtgttagagctctcccactagcaggtgatgaaaagccttagggtcaggtggcacttttcggggaaatgtgcgatgaacccctatttgtttatttttct

SEQ ID NO: 19 - DNA, 5543 bp, synthetic construct
  source 1..5543: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatggtaacgagggagactaaacttacttctgaacgggaagttgagagttcgatggcccaggcaagacacaatggaggcggtggaggcgagaaccatccctttacttcactaggaaggcaatccagcatttattccctaacccttgacgagtttcagcatgcgctctgtgaaaacggaaagaacttcggctctatgaatatggatgaattcttagtgtcaatctggaatgcagaggagaacaataataaccaacaacaggctgccgcagcagctggatctcactcagttccagcaaaccataacggattcaacaacaataacaacaatgggggagaaggtggagttggtgtgttctccggtggctctcgtggcaatgaggacgcaaataacaagagagggatagccaatgagtcatcactaccgcgtcaaggttccctcactctaccagcaccactctgtcgcaagacggtagacgaggtctggtctgaaatacaccgcggagggggatccggcaatggtggagacagcaatggtaggagttcatcatctaatggacagaacaatgcccaaaatggcggcgaaactgcagctagacaaccaacgtttggggagatgacactggaagatttcctcgttaaggctggggttgtaagggagcacccaacaaaccctaaaccgaatccaaaccctaaccagaatcagaaccccagttccgtgattcctgctgccgcacagcagcagctttacggagtgttccagggaacgggtgaccctagtttccctgggcaggcgatgggcgttggtgatcctagcggctatgcaaagcgtaccggagggggtggttatcagcaagccccacctgtacaagcaggggtctgctacggtggtggcgtcggatttggtgcgggcggtcagcagatgggcatggtaggacctctttcccccgtttcctctgatggacttggccatggacaggttgataatatcggtggacaatatggtgtggacatgggtggcttaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 20 - DNA, 5714 bp, synthetic construct
  source 1..5714: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcagggtaatgggaaatctcaccttcccgctgatttgcccggattggagtgctgacgacgagatgctcttgctggaagggctggaaatctatggccttgggaactgggcagaagttgcggagcacgtaggcaccaagtcaaaggaacagtgcctcgaacattaccggaacatctacttgaattctccgtttttccccttgcccgatatgtcccatgtggctggcaaaaacagaaaagaactgcaagctatggcgaagggccgaattgacgacaagaaggcagagcagaacatgaaagaagaatatcccttctccccgcctaaagttaaggtggaggacactcaaaaggagtctttcgtcgaccgctcattcggaggcaagaaacctgtgagtaccagtgttaacaattctttggtagaactttcgaactacaatcaaaagagggaggagttcgacccagaatatgacaacgacgctgaacagcttttggcggagatggagtttaaggagaatgacacccctgaagagcatgaactcaagttgagagtcctaagaatctactctaaacgcttggatgaaaggaaacgtcgaaaagagtttataattgagagaaatttgctttacccgaatcctttcgagaaggacctttcacaggaggagaaggtgcaatgtagacgcttggatgtctttatgagatttcattctaaagaagagcatgacgagttgcttaggaatgtggtatcagagtatcgaatggttaagagactcaaagacctgaaggaggcgcaagtagcaggctgtcgatcaacagctgaagcagagcgctacctaggtaggaagcgaaaaagggagaacgaggaaggcatgaacaggggtaaggaatcagggcagtttggacaaattgcaggcgaaatgggtagcaggccgccagtccaggccagtagcagctacgttaacgatctggatctgattggattcacggaatcacaactactttccgaatccgagaaacgactgtgctctgaggtcaagcttgtcccccccgtttatcttcagatgcaacaggtcatgtctcatgaaattttcaagggcaacgtgacgaaaaagagcgatgcttacagcctgtttaaaatcgatccaactaaagtcgatcgagtgtatgatatgttggtgaaaaagggcatcgcacaactgggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 21 - DNA, 5267 bp, synthetic construct
  source 1..5267: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatggccgcgaccctaggacgagatcaatacgtttatatggctaagctagctgagcaagccgagcgctatgaggaaatggttcagtttatggaacagctggttacgggagccactcccgctgaggagcttaccgttgaggagaggaacctgctttccgtcgcttacaagaatgtcatcggatcacttcgtgcagcgtggcgtatcgtttccagcatcgagcaaaaggaagaatccagaaagaacgacgagcatgtgagtctcgtgaaagactaccgttctaaagtggagtctgaactgagttcggtgtgctcgggaatactaaaactgcttgattctcatctcattcctagtgctggcgcaagtgagagtaaagtgttctaccttaagatgaagggcgattaccaccgctatatggcggagtttaagtctggtgacgagaggaaaaccgcggctgaagataccatgcttgcttacaaggctgctcaagatatagccgctgctgatatggctcctacccatccaattagactcggattggccttgaacttctccgtgttctactatgaaatcttgaactcgtccgataaggcatgtaatatggctaaacaagcttttgaagaagccattgcggaactcgacacacttggtgaagagtcatacaaagacagtactttgattatgcaattgttgagggataatcttactctttggacaagtgacatgcaagagcagatggatgaagcaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 22 - DNA, 4664 bp, synthetic construct
  source 1..4664: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcccgctacaccagcaccaggatattccacttaccaaccaagtacagccccaacctgaggagtccataggatcacctcaaggtataagtcaattaccatctatggacgatattgacgatagcttttgggaaaacttattcaaaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 23 - DNA, 4715 bp, synthetic construct
  source 1..4715: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagccataacatgcctccgccgctacctccccagcagcaccagattcaacacccgtacatgctgagccaccagccgagcccatttcttaacatgggtggtttgtaccaagaagatgatccaatggcaatgagaagaaacgatcttgagctgactctagagccggtctataattgcaatcttggatgtttcgctgctggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 24 - DNA, 5111 bp, synthetic construct
  source 1..5111: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatgtccgcaatcaagataactaatgtggccgttctgcacaacccagccccctttgtttcgccgttccagtttgagatatcctatgagtgtttgaatagcctcaaagatgatttggaatggaagctgatctacgttggttctgctgaggacgagacatatgatcagcttctagagtccgttctcgtggggccggtcaacgttggcaactacagattcgtgttccaagctgatccgcccgatccgagcaagattcaagaggaggatattattggtgttacggtcctcttgttgacttgtagttacatgggacaagaattcttgagggtggggtattacgtcaacaatgactacgaggatgaacaattgaaagaggaaccacccaccaaagtattgatcgataaggttcagcgtaatatcctgtccgataagcctagagtgacgaaatttcccattgattttcatccagaggaggagcaaacggccgcaacggctgcaccgccagaacaatcagacgagcaacagcctaatgtgaatggagaagcacaagttcttcctgatcagtcggtcgaacctaagccggaggagagtggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 25 - DNA, 5177 bp, synthetic construct
  source 1..5177: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatgagttccattaatatcaccaacgttacagtcctagataacccggccccttttgtgaatcccttccagtttgaaatttcttatgaatgcttgacatcactcaaggacgatcttgagtggaagctgatctatgttggttctgctgaagatgagacttacgaccaagtcctggaaagtgtcctcgtaggacccgtcaacgtcggtaactataggttcgtcctccaggccgattctcccgatcctttgaaaattcgtgaagaggacataattggggtgaccgtattgttactcacatgtagttacatggatcaagaatttatcagggttggctattacgtcaataacgattatgatgatgaacagcttcgagaagaaccacccacgaaggttttgatagacaaagttcaaaggaatatcctaactgacaaaccgagagtcacaaaattcccaataaacttccatcctgaaaacgagcagacactaggggatgggccagctccgactgaaccatttgccgattcggtcgtaaacggcgaagcccctgtgtttttggagcaaccgcagaagctccaggaaattgagcagttcgacgacagtgatgtcaacggcgaggctattgcactcctggatcaaccacaaaaccttcaggaaactggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 26 - DNA, 5576 bp, synthetic construct
  source 1..5576: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgtgtctctgtgggaaattgaacctctcaccacattccccatgtacccatccctcttcccacttcgcctcaagcgtccatggcacgccggaactagctccctccctgatggaaggggagatctagggagcggactaacgtggttgcggggaggaggcggggagcagcagggattgttgcctcttaattatccatccgtggggttgttcccttggatgcagcagcgactggaccttagtcaaatgggcactgacaacaatcaacagtatcaagcgatgcttgcggccgggctgcaaaatatcgggggaggcgacccattgcgccaacaattcgttcagctgcaagagccccatcatcaatacttgcaacagtcagcatctcacaattctgacctgatgctgcaacagcaacagcaacaacaggcgtcccgtcatctcatgcatgcccagacccagattatgtccgaaaacttgccgcagcagaatatgcggcaggaggttagcaatcagccagctgggcaacagcaacagctgcagcagcctgaccaaaatgcttatctaaatgcgtttaagatgcaaaatggccatcttcaacagtggcagcagcaatcggagatgccttccccatcatttatgaagtcagacttcaccgactctagcaataagtttgctacaacggcgagtccagcttccggtgatgggaatcttttgaatttttcgataaccggtcagtcagtgctcccagaacaattgactacagaggggtggagtccaaaggctagtaacacattctcggagccgttatctctcccacaagcgtaccctggcaagtccttagcactggagccgggtaacccccaaaacccttccctctttggcgttgaccctgactccggactcttcttacccagtactgttccaagatttgcgagttcatccggggacgcagaggcatccccgatgagtttaacagactctggatttcagaatagcttgtactcttgcatgcaggacactacccatgaattgttacatggtgcagggcaaatcaattccggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 27 - DNA, 5681 bp, synthetic construct
  source 1..5681: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagccaacaccagggcaatatgaaccacagcttcatgactgggcaagaccaaagcttcgggccactttcctcgttaaacggcttcgatctccaaagtttggctgtcacgggacaattacctcctcaaagcctcgcgcagcttcaagctgctggactcggccggccaacgttggccaaacctggtatgtccgtatcgccgttagtcgatcagcggtctattttcaattttgagaatccaaaaatcaggtttggagatggccacggtcagaccatgaataatggaaacttactacacggagtcccgacgggtagtcacatgaggcttcgaccagggcaaaatgtgcagtcatcaggtatgatgcttcctgtggctgatcaattgccaagaggaggtccctccatgcttccatcgcttggacagcagccgatcctatcttcttcagtgtcacggaggtctgatcttacaggcgctctagcagttcgcaactctatacctgaaacgaacagcagggtactgccaacgacccatagcgtctttaacaacttccctgccgatctgcctagatcctcttttcccttggcctctgcccctggcatttcggttccagtgtcggtcagctaccaggaagaggtgaatagtagtgatgccaagggaggatctagcgccgctacagcggggtttggcaacccaagctacgacatatttaacgacttcccgcagcatcaacaacataacaaaaacatttcaaacaaactgaatgattgggatctgaggaacatgggcctcgtgttctcatccaaccaggacgcggctacagccacggcaactgcggcattctcaacatcagaggcctactcctcatcatctacccagcgtaaacggagagagacagatgctactgtcgtcggagagcacgggcagaacctccaatctccaagtcgcaatctgtaccatcttaatcacgttttcatggatggtggttcagtaagggtgaaaagtgagcgtgtggcggagactgttacatgtccaccggcgaatactctctttcatgagcaatataaccaggaggacctgatgtcagcttttctgaaacaggagggaattccaagcgtcgataacgaatttgaatttgacggttattccattgacaacatccaggtgggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 28 - DNA, 4955 bp, synthetic construct
  source 1..4955: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcaggcagtttgacacacttaaagcagagaacgatcttcttcagactcacaatcaaaagcttcaagctgaaatcatgggactcaagaatcgagaacaaacagagagcattaacctcaataaagagacagaaggatcatgctcaaaccgttcggacaacagcagcgataatttaagactcgacatttctactgcccctccttcaaacgactcaactctgactggcgggcaccccccaccgccacagaccgttggcaggcatttctttcctccaagtcccgccaccgccaccacaaccacaacaactatgcagttcttccaaaattccagctctggacaatccatggtaaaagaggagaattctatttcaaacatgttttgcgcaatggatgatcattcaggattctggccgtggttggatcagcagcaatacaacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 29 - DNA, 4835 bp, synthetic construct
  source 1..4835: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcaaggagaaaaaatctttgtttggcttagatgttggccgtaaacgtcgcctcacctcgacaccatctttgggaactatggaggaaaacttgctgcatgatcaagaatttgatagaatgaaggacgacatggagatgcttttcgcagccgctattgatgacgaggccaataactcgatgccgaccaaggaagaacaatgcttagaagccatgaacgtgatgatgcgggacggcaaccttgaggccgcgctcgacgttaaagtcgaggatttagtcggcagccctctggactgggatagtcaagatctccatgacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 30 - DNA, 4961 bp, synthetic construct
  source 1..4961: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcaaagagaagagaaaagaaatagaggaggcaatatcgaaaaagaggcagcgccccatcgaccagggaaagcggaacgtcgaagattatggggacgagtcaggttacggaaacgatgtcgctgcttcttcttcggcactcattggaatgtcacaagagtacacatacggtaacatgtcagaatttgagatgagcgaactggacaaattggcaatgcacatccaagggctgggcgataattctagcgctagagaggaagtcttaaatgttgaaaagggtaatgatgaagaagaggttgaggaccagcagcaaggttatcataaagagaacaatgagatctatggggagggattctgggaggatcttcttaacgaagggcagaatttcgacttcgagggcgaccaggagaatgtggatgttggcagttcaagtcatactaatggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 31 - DNA, 5042 bp, synthetic construct
  source 1..5042: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgacggcgcagacgttctgaaaatgagtcagttaaaggctaggaagaagcatctccggttccaacaaagtaaaattcacgattggggattggttgcgttagaacctattgaggcagaagatttcgtgatagagtacgtgggcgaactcataaggtcgtcaatttcagaaatccgggaacggcagtacgaaaaaatgggcattggatcctcttacctcttcagattagacgatggatatgtcttagacgccacaaagcggggaggaatcgctagattcataaaccactcatgtgaaccaaattgttacacaaagatcatctccgtggaaggcaagaagaagatttttatctatgctaagagacatatcgacgctggagaggagatctcttacaactataagttcccgttggaagatgataagataccttgtaactgtggagccccaaacgtttactgcttttgtgagcaagtaccgtggattgccaagctgaaacgcaggacttggttttcccggaggaacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 32 - DNA, 4652 bp, synthetic construct
  source 1..4652: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcaccgtgatgtgggaacaggatgcagccccctttgcaggcgcagccgatgactttccagcttttaacgaggaggaattagcgtggctgatggagctgctccctcaaagcggatctgtgggggggaccattggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 33 - DNA, 5186 bp, synthetic construct
  source 1..5186: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagctcgtccgtgtataaaccgacgtctaaaagtagctcgtcaagttcatccaatcaagatttctcagtgccgggaagtagcatctctaaccccccacctcttaaaaagcccttgtcagcgcccccccagagcaacaagattcaaccattcagcaaaatcaacggaggggacgcatcagtaaatcagtggggctcgaccagcactattagcgagtacctcatggataccctcccagggtggcatgtagaagattttttggactcaagccttccaacgtacggatttagtaagagcggggacgatgacggagttttgccatatatggagccggaggacgataataatactaagcgaaacaataataacaataataacaacaataacaacacagtgtctctgccgagtaagaaccttggcatatgggtgcctcagataccgcagacattaccatccagctatcctaaccagtatttctcacaggacaacaacatacaattcggcatgtacaataaagagacctcacctgaagtagtttctttcgccccaattcagaacatgaagcaacaaggacagaacaataaaagatggtatgatgacggaggatttaccgtccctcaaatcaccccgcctcctttaagctccaacaagaagttccgcagcttttggggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 34 - DNA, 5423 bp, synthetic construct
  source 1..5423: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatggagtcggagtttcagcaacatcacttcctgctacatgaccatcaacaccaacgccctcggaattccggtctgattaggtatcagagtgcgccttcctcttatttttcctcattcggtgagtctattgaggaattcctagacagacctacctcgcctgaaaccgaaagaattttgtctgggttccttcagactaccgacacctccgataacgttgactcgttccttcaccatacatttaacagtgacgggacggagaagaagccacccgaggttaagacagaggatgaagatgctgaaattccggttactgcgaccgcgacggccatggaggttgtagtgtccggtgatggcgaaatctctgtaaaccctgaagtatctataggctacgtggcttctgtatcgcgaaataagaggccccgagaaaaagacgacagaactcccgtcaacaatctggctagacacaactcaagccccgcgggacttttttcaagcattgatgtcgagacagcgtacgctgctgtgatgaagtcaatggggggatttggaggttctaacgtcatgtctactagtaacacggaagcgagttctctgacaccaaggtcgaagctccttccgccaacgagtcgcgctatgtcgccgatctcagaggtagacgttaagccgggtttcagttcgaggttacctcctcgtactttatctggcggattcaacaggtcatttggcaacgagggttcagcttcaagtaagctgacagctttggcacgcacccagtcgggcgggttggatcagtataagacaaaggatgaagatagcgcctcgcgtcggcctcctcttgcacatcatatgtctcttcctaaatctttatccgacatagagcagttgttatccgattccatcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 35 - DNA, 5087 bp, synthetic construct
  source 1..5087: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgacatccagcaaggatccgcctacgcgagctttctttctaagaagagttcaagaatcgaggatcaacttcgcttctgcactgatcaatttcagaagttggctgaggacaagtaccaaaagtctgtttcattggagaatcttcagaaaaaacgtgctgatataggtaacggcctggagcaagctcggagtaggcttgaagaatcacattctaaagttgaacaatcacggctcgattacggtgcacttgaattggagctggaaatagagcgatttaacagacggcggattgaagaagaaatggaaatcgccaaaaaaaaggtgtcccgtcttaggtcgcttatcgaaggttcctctgcgatacagaaactgcgacaggaactttctgagtttaaagagattctaaagtgtaaggcgtgcaatgacaggccgaaagaagttgtgatcacaaagtgttaccacctattctgcaacccatgtgtccaaaagttgactggtactcgtcagaagaagtgcccaacttgttccgctagttttggcccaaatgatataaaacccatctacatcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 36 - DNA, 4673 bp, synthetic construct
  source 1..4673: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcctctgcgtggacatatataagcgagcgtttccaagatcggtgaacaagggaagatcatcatacgctggcggtagtggaggcgttccagggatttcttataactctaaggttcgcgattacattctctggggggttaccgaagtgattccaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 37 - DNA, 4715 bp, synthetic construct
  source 1..4715: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagccaaccgggcacagaggacgcagaggccgtagcattagggttgggtctgtcagattttccttcggctggaaaggccgtcctggacgacgaagatagcttcgtctggccagcggcgtcgttcgacatgggggcctgttgggcaggagccggttttgctgatccggatccggcttgcattttcttgaatctacctggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 38 - DNA, 4877 bp, synthetic construct
  source 1..4877: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgggaggagcgcgtgtctaaacttcgcagattcggcatggaggctcagaattccagagtctacgtgcgcgaaagatattcagaaggcagcggcggaggcggctctcgcgttccaagacgagatgtgtgatgctacaactgatcatggattcgatatggaggagacactagtcgaggctatctacaccgctgaacaatcggaaaatgctttttatatgcatgatgaggctatgtttgagatgccttcgctcctcgcgaacatggcggaagggatgctgttgcctcttccgagtgttcaatggaaccacaatcacgaagtcgacggtgacgatgacgatgtctcgttgtggtcgtatggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 39 - DNA, 4766 bp, synthetic construct
  source 1..4766: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcagcagcgatatgtttgacgtagatgagctgctaagagatctcaacggggatgatgttttcgccggcctcaaccaggacagatacccgggcaatagcgtggcgaatggctcctacaggcctgagtcacaacagtcagggtttgacccacttcagtctcttaattacggtatcccacctttccagctcgaaggtaaagacgggaacggattcttcgacgacctatcttacttggatcttgagaacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 40 - DNA, 5913 bp, synthetic construct
  source 1..5913: mol_type = other DNA
tcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcagcagcgatatgtttgacgtagatgagctgctaagagatctcaacggggatgatgttttcgccggcctcaaccaggacagatacccgggcaatagcgtggcgaatggctcctacaggcctgagtcacaacagtcagggtttgacccacttcagtctcttaattacggtatcccacctttccagctcgaaggtaaagacgggaacggattcttcgacgacctatcttacttggatcttgagaacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtg

SEQ ID NO: 41 - DNA, 6769 bp, synthetic construct
  source 1..6769: mol_type = other DNA
cgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgccggggcgcgccgatcatgagcggagaattaagagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataaagagactcttatcaagaatcccatctcttgcttgcttttttttgttgtcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtgattaggagacgtgtttcattaataaaatataagatcaatacattgttagtagtgataaactatgtacaaattgtattgattgtaaaagaaacacaataggttccttttttctacaatatattgtgacagactctctgttttaacgaatgaattaaatttgtcaacgaaaaaattaaagaatgatgtgatcactttgcaccttggttcatcttcagaggcttctgcatgttccatgcattgaggctctctacggtcacaggttgtgagccgttgttaaagacaaacaaatgctcgtgacttgcgaagatcgatccactagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagctccaccgcggtggcggccggccgtctagtggatcgattccccttcgcaagacccttcctctatataaggaagttcatttcatttggagaggacctgcaggatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgctggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgactcgaggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtgttagagctctcccactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaa

SEQ ID NO: 42 - DNA, 6181 bp, synthetic construct
  source 1..6181: mol_type = other DNA
ctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgccggggcgcgccgatcatgagcggagaattaagggagtcacgttatgacccccgccgatgacgcgggacaagccgttttacgtttggaactgacagaaccgcaacgttgaaggagccactcagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaatccaaataatctgcaatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataaagagactcttatcaagaatcccatctcttgcttgcttttttttgttgtcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtgattaggagacgtgtttcattaataaaatataagatcaatacattgttagtagtgataaactatgtacaaattgtattgattgtaaaagaaacacaataggttccttttttctacaatatattgtgacagactctctgttttaacgaatgaattaaatttgtcaacgaaaaaattaaagaatgatgtgatcactttgcaccttggttcatcttcagaggcttctgcatgttccatgcattgaggctctctacggtcacaggttgtgagccgttgttaaagacaaacaaatgctcgtgacttgcgaagatcgatccactagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagtctttcaattgtgagcgctcacaattctttctcttccctttcttctttctagctccaccgcggtggcggccggccgtctagtggatcgattccccttcgcaagacccttcctctatataaggaagttcatttcatttggagaggacctgcaggatggaagacgccaaaaacataaagaaaggcccggcgccattctatccgctggaagatggaaccgctggagagcaactgcataaggctatgaagagatacgccctggttcctggaacaattgcttttacagatgcacatatcgaggtggacatcacttacgctgagtacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgggcatttcgcagcctaccgtggtgttcgtttccaaaaaggggttgcaaaaaattttgaacgtgcaaaaaaagctcccaatcatccaaaaaattattatcatggattctaaaacggattaccagggatttcagtcgatgtacacgttcgtcacatctcatctacctcccggttttaatgaatacgattttgtgccagagtccttcgatagggacaagacaattgcactgatcatgaactcctctggatctactggtctgcctaaaggtgtcgctctgcctcatagaactgcctgcgtgagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttctgaggagccttcaggattacaagattcaaagtgcgctgctggtgccaaccctattctccttcttcgccaaaagcactctgattgacaaatacgatttatctaatttacacgaaattgcttctggtggcgctcccctctctaaggaagtcggggaagcggttgccaagaggttccatctgccaggtatcaggcaaggatatgggctcactgagactacatcagctattctgattacacccgagggggatgataaaccgggcgcggtcggtaaagttgttccattttttgaagcgaaggttgtggatctggataccgggaaaacgctgggcgttaatcaaagaggcgaactgtgtgtgagaggtcctatgattatgtccggttatgtaaacaatccggaagcgaccaacgccttgattgacaaggatggatggctacattctggagacatagcttactgggacgaagacgaacacttcttcatcgttgaccgcctgaagtctctgattaagtacaaaggctatcaggtggctcccgctgaattggaatccatcttgctccaacaccccaacatcttcgacgctggtgtcgcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagatcgccgtgtgactcgaggttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtgttagagctctcccactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgc

SEQ ID NO: 43 - DNA, 5777 bp, synthetic construct
  source 1..5777: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcaaaactactggagcactgctagacgcggacattggaaagacgtctgccacgtacggaacgatctcatctgatgtgacacacggagagatggtggtggatgtaaccattgaagattcatattctacagagtctgcgtgggtgagatgtgacgactgcttcaaatggcggcggatcccggctagtgtcgttggttctatagacgaatcctccagatggatctgcatgaataattctgataaaaggttcgcagattgttcaaagtcacaggagatgtccaatgaagagatcaacgaggaactcgggatcgggcaagatgaagccgatgcgtacgattgcgatgccgcaaagagaggtaaggagaaggagcaaaaatccaagcgtctgaccgggaaacaaaaagcctgtttcaaggcgatcaagactaaccagttcctccaccggaaccgcaagtcgcaaactatcgatgaaatcatggtttgtcattgtaagcccagccctgatgggcgccttggctgcggcgaagaatgcttgaacaggatgctgaacattgaatgcctacaggggacttgcccggcaggagatctctgttcaaatcaacaatttcagaagcgaaagtatgtgaagtttgaacgcttccagagcggaaaaaagggctatggattaagactactggaggatgttagagagggacaattcttgatagaatatgtgggtgaggtactggatatgcaatcttacgagacaagacaaaaggaatacgcattcaaaggccaaaagcatttttatttcatgacactaaacggtaacgaagtcatcgacgcgggagctaaaggtaacctcggaaggttcatcaaccacagttgtgagccgaattgccgaaccgagaagtggatggttaatggagaaatatgcgtgggaattttctcaatgcaagacctgaaaaaaggacaggaattgacgtttgattataactacgtgcgagtgttcggggctgcagcgaagaagtgttattgtgggtctagccattgccgaggctatattgggggcgatcccttaaatggtgatgtaatcatccagtccgattctgacgaagagtacccagaactggttatccttgatgacgacgagtcgggagagggaatactcggtgcaactagcagaaccttcacagacgacgccgatgaacagatgccccaatcattcgagaaggtgaatgggtacaaggaccttgccccagacaatacgcaaacgcaaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 44 - DNA, 5477 bp, synthetic construct
  source 1..5477: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagctcattgagcggcggttcatgttctctccttatgaacgattgttcccaatatgacgtcgaaggcttcgaaaaagaaagtcactacgaagtagaggagttgaagccagagaaggtgatgaatagttctaacttcggtatggtcgcgaagatgcacgactttcctgtcaaagaagaggtgcccgctgggaactccgaattcatgcgtaaacggaagccaaacagggaccttaacaccataatggacagaactgtgttcacatgtgagaatctcggatgcgctcactcagaaattagcaggggtttccttgaccggaattctcgcgataatcatcagttggcttgtccacacagagatagcagactcccttatggggctgcaccgagccgattccacgtaaatgaggtgaagccggtcgttggcttcccgcaacctcgccctgttaacagcgttgcacagccgattgatttaaccgggattgttccggaggacggtcagaagatgataagcgaactcatgtctatgtatgataggaatgtgcagtcaaatcagacgtccatggtcatggagaatcaaagcgtttcactacttcagccaacagtgcacaaccatcaagagcaccttcagttcccaggtaatatggtcgagggttctttcttcgaggaccttaatatcccaaaccgggcgaacaataacaacagttccaacaaccaaacgttcttccagggaaacaacaacaataataacgtttttaaattcgacacagctgatcataataacttcgaggctgcacacaataataacaataattcgagtgggaaccgattccaattggtttttgattcaactccattcgatatggcgagtttcgattacagggacgatatgagcatgcctggggtcgtgggaacgatggacggaatgcaacaaaagcagcaggatgtctcaatttggtttggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 45 - DNA, 5165 bp, synthetic construct
  source 1..5165: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcagatataaggcttgcgagactacagagcccgacgcgatacgtcaacagttccccttttacaacgaaatccagtccatcttcgaagctagaatgcagagaatgctttggtctgaagcgactgaaccgtctacgagttcaaagcgaaaacaccaccaattttcatctgatgatgaagaggaggaagttgacgagcctaaccaagatatcaacgaagagctgttgtctctcgtggagactcagaagcgggaaaccgaggttattacgaccagtacttctactaatccccggaaaagagcgaagaaaggaaagggcgtagcgagtggaacaaaggcggaaacagccggcaacacattaaaagatatcctcgaagaattcatgagacagacagtaaagatggagaaggagtggcgcgacgcctgggaaatgaaagaaatcgagcgtgaaaagagggaaaaagagtggaggcgcagaatggcggaactagaggaggagagggcagcgacagagaggcgatggatggagagggaagaagaacggcggcttcgggaggaggcccgggcccaaaagcgggattcgctgattgatgctctcctcaataggctaaaccgggaccataatgatgatcaccataaccaaggctttggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 46 - DNA, 5777 bp, synthetic construct
  source 1..5777: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcctggagagttcagatggtggtaaggatggcggcagctccgtggtggggaccggagtttccggaacggtggggggatcaagtatatcaggtttggttcccaaagatgagtctgtcaaagttctagcagaaaacttccaaacctctggggcctacattgcaagagaagaagctttaaaacgtgaggagcaggccggtcgtcttaaatttgtctgctactcgaatgactcgattgacgaacacatgatgtgccttattggactcaagaacattttcgcccgtcaacttccgaatatgccaaaggagtacattgtgaggttgttgatggatcggaagcacaagagcgtcatggttctccggggtaatctcgtcgttggtggcattacttatagaccctaccattctcaaaagtttggagaaattgccttctgtgccattaccgcagatgagcaggttaaaggatacggcaccaggctcatgaatcatcttaagcagcacgcaagggacgtcgatggactaactcactttttgacttacgctgacaataacgcagtcggttacttcgtaaagcaaggctttacaaaagaaatttacctcgaaaaggacgtatggcatgggttcatcaaggattatgacggtggactgctcatggagtgcaagatcgacccaaagcttccatacactgatttgtctagtatgattagacaacaacgcaaggctatcgacgaaaggatccgtgaactgtctaattgccaaaacgtgtatccaaagattgagtttttgaagaacgaagctggcatcccccgcaagatcatcaaggtggaagaaatccgcggacttagggaggcagggtggaccccggaccaatggggacatacgagattcaagcttttcaacgggtcggcagatatggtgaccaaccaaaaacaacttaatgccctcatgagagcattgcttaagacaatgcaggaccacgcagatgcctggccattcaaagaaccagtcgactctagggacgtgccagactattatgacattattaaagatcctattgacctcaaggtgatcgcgaagcgagtggaatctgagcagtattatgtgactcttgatatgttcgtagccgacgccaggcgcatgtttaataactgtagaacctacaactctcccgatacaatttattataagtgcgcaacacgtctggagacacattttcactcaaaggttcaggcgggtttgcaaagcggcgctaaaagccaaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 47 - DNA, 5063 bp, synthetic construct
  source 1..5063: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgcagcgacaaatctcaagggaattggacctcaaaccattccagatatgggtataatgaaagagtttggaagccctttaggattttccttccaagatttggacctatatgggggaggcggaggtgaccaacttgaactccaacagaatatggagaaaccgcccttggacgggtttatgcctatgaaccacgaaaatctgtgcttgggtaagaagcgccctaatccatactccggtaacaacggcaagtctccccttatgtggagtgatgatcttcggctccaggatctgggatcttgcttgcaggatgatccctttaagggcgaccatcaccatcagattcagattgccccaccatcattggatcgtggtactgagatggatcctatgtctgaaatctacgactcaaagcccgaagaaaaaaagtttgacgcctcaatgaagctggaacggccttctcctcgccgggcccctctgggagaaagaatgtccccgatgatcacaacagggacaatggctcagggtcggtccagtccatttggcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 48 - DNA, 5009 bp, synthetic construct
  source 1..5009: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagctttccggcaagaaagaccaaggagttccaggccgtgcacaaagtttttggtgtatccaatgtgcagaaaatggtgagaaccgtgcgtgaagaggatcggactaaactgagcgattctttgacttgggaagctctctggcggcaaaaagaccctgtgcttggttcatatggagagtatcgacgcatctgtgaggaactgaagctgtacaagtccctagtccataaccagcccctaattggctgggataataaccagcgggttttcaataacaattccaataataagaacggattggctatgactaattcttcaggttccgggggcttttcggttaacaataatggagttggagtcaacagggaaatagtgaatggcggatatgcctcaaggaacgttcaaggtggatgggagaatctgaagcacgaccaaagacagcaatgctacgccgtcattaataatggattcaaacaacattatctgcccttgggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 49 - DNA, 4838 bp, synthetic construct
  source 1..4838: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgcaaaacagctagagcagctgtacgatagtctacgccaggaatatgatgtggtcagccgcgaaaaacagatgctccatgatgaagtgaaaaagctacgggctcttctacgtgatcaagggttgatcaagaagcagataagcgcgggcaccatcaaagtgtcaggcgaagaggacacagttgaaatttcttctgttgtcgttgcacatcctcggaccgagaacatgaacgctaatcaaattacaggtggaaaccaagtctacggccaatacaataatccgatgctagtggccagcagcgggtggccaagttatcccggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 50 - DNA, 5105 bp, synthetic construct
  source 1..5105: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcggtggagtagctccctcagatgatgccgtggacgaggaagtaaccgataccgaatggtttttcttggtttctatgactcaatccttcgcatgcggcgctggattggccggtaaggcttttgcaggaggaggtggctccggcggtggcggatctggtggtgtggctccctcagacgatgcggtcgacgaagaggtcacagatactgaatggttctttcttgtctctatgacacaatcttttgcttgcggcgccggtctggcgggtaaggcgttcgccggcggaggcgggtctggaggaggcggtagcggcggggtggcgccatccgacgacgcggtggacgaggaagtgacagacacggagtggttctttttagtttccatgactcagtcatttgcttgcggagccgggctggccggaaaagcattcgcaggcggaggtggttcaggtggaggtggctcgggaggagtggcaccatctgatgatgctgtggatgaagaggtaacagacacggaatggttttttttggtttcaatgactcagtcattcgcctgcggtgctgggctagcaggaaaggcattcgctggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 51 - DNA, 5072 bp, synthetic construct
  source 1..5072: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcattgatgtcgctgcagctggtcacggagatggcgatatgatggatcagcaacacgcgacggaatggacctttgagcgcttgctagaagaggaggcgttgactactagcacaccacctcccgttgttgtggttccaaacagctgttgttcgggagctttgaatgccgacagacctccagttatggaggaggctgttaccatggctcctgccgccgttagtagtgctgtcgtgggggatccgatggagtacaacgcgatactgcgcaggaaacttgaagaagatttggaagcgttcaagatgtggcgggccgatagttctgtcgttacttcagatcagaggtcacaaggatctaataaccataccggcgggagttctatacggaataacccagtccaaaacaaattgatgaatggggaggacccgatcaataacaatcacgctcaaactgccggccttggggttcgcctggcgacgtcaagtagctctcgggatccatctcctagcgatgaggacatggacgaggaggttgaaatattggggtttaagatgggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 52 - DNA, 5177 bp, synthetic construct
  source 1..5177: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcccagtgacggagagtttgcttgaacttgttctcagaaataggctacaggaatggctggtagaaatggtcatggaggggcacaaatctaccggtcgggacgatttgggccaaggagcaattcatctttgctcgttcctcggatacacgtgggccattcgactgttctcattatcaggatttagcctggatttcagggattcatcaggatggactgctctccattgggcggcatatcatggtcgcgagaggatggtggcgactcttctgtccgcgggggcgaatcctagtttagttaccgacccgacaccagaaagcccagctggtcttaccgctgctgatctggcagcgcgtcaagggtatgatgggctcgctgcataccttgcagagaagggcttgaccgctcactttgaagcgatgtccttgtcaaaggatacggagcagtccccatcaaaaactagactcactaagttgcaaagtgaaaaatttgaacacttgagcgaacaggagctatgcctaaaggagtcactcgcagcgtacagaaatgcagccgacgcggcctcaaacatccaggcggctctacgcgaaaggactcttaaacttcaaacaaaagcaattcagctcgccaacccggagatagaggcatctgaaatagtcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 53 - DNA, 5051 bp, synthetic construct
  source 1..5051: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagccctccaccgtcctactactctatggatcacaaggagtacgcttatgggcatgccactaaggaggtgcatggagaacatgcgttcttttctgatggaacagagcgtgaacatcaccacgccgccgccggacatgggcaatggcaattcaaacaactgggaatggaaccgaagcaatcgacgactcccctgttccctggtgcaggctacggccataccgctgcttctccatacgctattgatctgtcaaaggaggacgacgacgagaaagaaaggaggcaacagcaacagcaacagcaacaacattgtttcttactcggggcggatctaagacttgaaaagcctgctggccatgatcatgcagcagcggctcagaagcctctgagacactttttcgatgaatggccacacgaaaagaactcaaagggttcctggatgggattggagggcgaaacccagctatcaatgtctattccaatggccgctaatgaccttccgattactacaacgtcacgataccataacgatgagggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 54 - DNA, 4718 bp, synthetic construct
  source 1..4718: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgagcctgaaccggttagagtgaccgcgaagaggagagcgtccccagagccagccagttcatctggcaacggcagcatgaaaaggcggagaaaggccgtgcagaaatgcgatggggaaatggccagtcgaagctctgttatgcaagtcggttgccaaatcgaacagctcacaggcgtgcaccagcttcttgtcatcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 55 - DNA, 4362 bp, synthetic construct
  source 1..4362: mol_type = other DNA
cgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgccggggcgcgccgatcatgagcggagaattaagagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgacttcgaaagtttatgatccagaacaaaggaaacggatgataactggtccgcagtggtgggccagatgtaaacaaatgaatgttcttgattcatttattaattattatgattcagaaaaacatgcagaaaatgctgttatttttttacatggtaacgcggcctcttcttatttatggcgacatgttgtgccacatattgagccagtagcgcggtgtattataccagaccttattggtatgggcaaatcaggcaaatctggtaatggttcttataggttacttgatcattacaaatatcttactgcatggtttgaacttcttaatttaccaaagaagatcatttttgtcggccatgattggggtgcttgtttggcatttcattatagctatgagcatcaagataagatcaaagcaatagttcacgctgaaagtgtagtagatgtgattgaatcatgggatgaatggcctgatattgaagaagatattgcgttgatcaaatctgaagaaggagaaaaaatggttttggagaataacttcttcgtggaaaccatgttgccatcaaaaatcatgagaaagttagaaccagaagaatttgcagcatatcttgaaccattcaaagagaaaggtgaagttcgtcgtccaacattatcatggcctcgtgaaatcccgttagtaaaaggtggtaaacctgacgttgtacaaattgttaggaattataatgcttatctacgtgcaagtgatgatttaccaaaaatgtttattgaatcggacccaggattcttttccaatgctattgttgaaggtgccaagaagtttcctaatactgaatttgtcaaagtaaaaggtcttcatttttcgcaagaagatgcacctgatgaaatgggaaaatatatcaaatcgttcgttgagcgagttctcaaaaatgaacaataaagagactcttatcaagaatcccatctcttgcttgcttttttttgttgtcttccctttgatagggtttgtttttcttgtttcagtgactttctatgttaaaagataatgtcagtaaaaggatttggttttctattattctgaatcgattacggaagattcttgcttaattccaatctatacaagtatcgtgaaataatgaccgtttatgtgattaggagacgtgtttcattaataaaatataagatcaatacattgttagtagtgataaactatgtacaaattgtattgattgtaaaagaaacacaataggttccttttttctacaatatattgtgacagactctctgttttaacgaatgaattaaatttgtcaacgaaaaaattaaagaatgatgtgatcactttgcaccttggttcatcttcagaggcttctgcatgttccatgcattgaggctctctacggtcacaggttgtgagccgttgttaaagacaaacaaatggttagagctctcccactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtggctctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaa

SEQ ID NO: 56 - DNA, 5006 bp, synthetic construct
  source 1..5006: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgagattttgagcctcccggaggatttggacttgtgcgcggcaggaggaggtgatggaccatctctaagcgacgagaacgatgaggaactgttttcaatgttcctggatgtcgagaagctaaactcaacatgtggggcaagtagtgaagccgaggccgaaggcggcggtggctctggcgggggtgggtccggaggcatgatgatgaacttcggcggaatgccgcatcagtttgggggtaaccaacaaatgttccagaataatcaagccatgcaatccatgctcgcagcacatcagctccaacagcttcagctgcatccacaagcccagcagcagcaggtcctccacccccagcatcaacagcagcaacctcttcatccccttcaagctcaacaactacagcaggccgcccgggatctgaagatgaagagtccaatggggggtcagtcccagtggggtgacggaaagtccggaagctccggaaatggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 57 - DNA, 5324 bp, synthetic construct
  source 1..5324: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagctgctgtgccaattgcaagggaaattgccttagcgcagatttcccatgtacgtgcgcacgagaaacctcaggcgaatacgcttatacgaaagagggcctgcttaaagagaaatttttagatacgtgcctcaaaatgaaaaaggagccggatagctttccaaaagtgtactgtaaagattgtccgcttgagcgcgaccatgataaagggacctatggtaaatgcgatggtcatcttatccgtaagttcattaaggaatgttggcgtaaatgcgggtgcgatatgcagtgcggaaatagagtcgttcagcggggcattagatgtcaattgcaagtctactttacgcaggaaggaaagggctgggggttgaggacacttcaggatttgccaaagggaactttcatctgcgaatatataggagagattctgactaacacagaattgtacgatcgtaatgtcaggtcatctagcgaaagacacacataccctgttactctggatgccgattggggatccgaaaaagatctgaaagacgaggaggccctatgtcttgatgctacaatttgtgggaatgtagccaggttcatcaaccacaggtgtgaggacgccaatatgatagatatccccattgaaattgaaacaccagaccgtcattactatcatattgcttttttcactctaagggatgtcaaggcgatggatgagttgacgtgggactacatgatcgatttcaacgataaaagtcaccctgttaaagcttttcgttgctgttgcggatctgagtcctgtagagacaggaagatcggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 58 - DNA, 4952 bp, synthetic construct
  source 1..4952: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagctgggagaaaatgcagcaaggcaaggaggtcaaggaagaggcgtctgatatggtaacctctcagtcccatagtcacacacattcttggggggagacacgtaccccggagagtgaaattgttgataacgacccattcccagagcttgatagctttccagcatttcagcctgctcctcctccagctaccgccatgatggtccctaagaaagagtcaatggacgacgcaaccgcggcggccgcagctgccgcaactattccgcggaacaattcttctttgttcgttgacctttcctatgatgacatccagggcatgtattctggcttggatatgcttccgcctggcgacgatttttacagttcactattcgcatctcctcgcgttaagggtaccaccccaagagctggggcaggaatgggaatggtgccgtttggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 59 - DNA, 5123 bp, synthetic construct
  source 1..5123: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatgacagaccaaggtttggagggatcaaacccggtagatctctccaagcatccatcgggcatcgttccgacccttcaaaacatagtatccaccgtgaacttagactgcaagctcgatcttaaagctattgctctccaggcgaggaacgctgaatacaatcctaaacgtttcgctgctgtgatcatgagaattagagagcctaagaccacagctcttatctttgcgtcgggtaagatggtttgtactggcgccaagtctgaagatttttctaaaatggcagcgcggaaatacgcacgtatcgtccaaaagttaggattcccagcaaaatttaaggatttcaagattcaaaatatcgtcgggtcgtgtgacgttaagtttccaatccgactggagggactagcgtattcccacgctgcgttttctagttatgagcctgaacttttcccaggactcatctatcgtatgaaggttccaaaaatagttctcctcatctttgtgtctggcaaaattgtcattactggtgcaaagatgagagatgagacatataaggccttcgagaatatctacccagtgctgagcgaattcagaaagatacaacaaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 60 - DNA, 4943 bp, synthetic construct
  source 1..4943: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcgaaataaactatcatagcaccggtctaatggattccgcgcataatcatttcaaacgagcacgcttattcgaagatctggaggatgaggacgccgaggtcatttttcccagcagcgtgtacccttctccacttcctgagtccacagtgccggcaaacaaagggtatgccagctctgcgattcagacactctttacaggaccagtcaaagccgaggaaccgacacctactccaaaaatcccaaagaagcgaggtagaaaaaagaaaaacgccgatccagaggagattaactcgtcagccccacgagatgatgacccagaaaataggagcaaattctatgagagcgcttctgcgcggaaacggaccgtcaccgccgaggagcgggaaagggcaattaacgccgcaaagacattcgagcctacaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 61 - DNA, 4841 bp, synthetic construct
  source 1..4841: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatgaacgatgcagacaccaaccttggaagctcattttccgacgatacccattcggtattcgaatttcctgaattggacttgtcagatgaatggatggatgatgatttggtatccgcagtaagcggcatgaaccagagctatgggtaccagacttcagatgttgctggagccttgttttcaggttcaagcagttgcttcagtcatccagaatccccatcgactaaaacctacgtagcagctacggccacagcctcggcagacaaccagaacaagaaagagaagaaaaaaatcaagggccgggttgcattcaagactagaggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 62 - DNA, 10252 bp, synthetic construct
  source 1..10252: mol_type = other DNA
gtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgctcatgatcggcgcgcctgcagtgcagcgtgacccggtcgtgcccctctctagagataatgagcattgcatgtctaagttataaaaaattaccacatattttttttgtcacacttgtttgaagtgcagtttatctatctttatacatatatttaaactttactctacgaataatataatctatagtactacaataatatcagtgttttagagaatcatataaatgaacagttagacatggtctaaaggacaattgagtattttgacaacaggactctacagttttatctttttagtgtgcatgtgttctcctttttttttgcaaatagcttcacctatataatacttcatccattttattagtacatccatttagggtttagggttaatggtttttatagactaatttttttagtacatctattttattctattttagcctctaaattaagaaaactaaaactctattttagtttttttatttaataatttagatataaaatagaataaaataaagtgactaaaaattaaacaaataccctttaagaaattaaaaaaactaaggaaacatttttcttgtttcgagtagataatgccagcctgttaaacgccgtcgacgagtctaacggacaccaaccagcgaaccagcagcgtcgcgtcgggccaagcgaagcagacggcacggcatctctgtcgctgcctctggacccctctcgagagttccgctccaccgttggacttgctccgctgtcggcatccagaaattgcgtggcggagcggcagacgtgagccggcacggcaggcggcctcctcctcctctcacggcaccggcagctacgggggattcctttcccaccgctccttcgctttcccttcctcgcccgccgtaataaatagacaccccctccacaccctctttccccaacctcgtgttgttcggagcgcacacacacacaaccagatctcccccaaatccacccgtcggcacctccgcttcaaggtacgccgctcgtcctcccccccccccctctctaccttctctagatcggcgttccggtccatggttagggcccggtagttctacttctgttcatgtttgtgttagatccgtgtttgtgttagatccgtgctgctagcgttcgtacacggatgcgacctgtacgtcagacacgttctgattgctaacttgccagtgtttctctttggggaatcctgggatggctctagccgttccgcagacgggatcgatttcatgattttttttgtttcgttgcatagggtttggtttgcccttttcctttatttcaatatatgccgtgcacttgtttgtcgggtcatcttttcatgcttttttttgtcttggttgtgatgatgtggtctggttgggcggtcgttctagatcggagtagaattaattctgtttcaaactacctggtggatttattaattttggatctgtatgtgtgtgccatacatattcatagttacgaattgaagatgatggatggaaatatcgatctaggataggtatacatgttgatgcgggttttactgatgcatatacagagatgctttttgttcgcttggttgtgatgatgtggtgtggttgggcggtcgttcattcgttctagatcggagtagaatactgtttcaaactacctggtgtatttattaattttggaactgtatgtgtgtgtcatacatcttcatagttacgagtttaagatggatggaaatatcgatctaggataggtatacatgttgatgtgggttttactgatgcatatacatgatggcatatgcagcatctattcatatgctctaaccttgagtacctatctattataataaacaagtatgttttataattattttgatcttgatatacttggatgatggcatatgcagcagctatatgtggatttttttagccctgccttcatacgctatttatttgcttggtactgtttcttttgtcgatgctcaccctgttgtttggtgttacttcctgcaggtagatcgctcgtcgacatggataagaagtactctatcggactcgctatcggaactaactctgtgggatgggctgtgatcaccgatgagtacaaggtgccatctaagaagttcaaggttctcggaaacaccgataggcactctatcaagaaaaaccttatcggtgctctcctcttcgattctggtgaaactgctgaggctaccagactcaagagaaccgctagaagaaggtacaccagaagaaagaacaggatctgctacctccaagagatcttctctaacgagatggctaaagtggatgattcattcttccacaggctcgaagagtcattcctcgtggaagaagataagaagcacgagaggcaccctatcttcggaaacatcgttgatgaggtggcataccacgagaagtaccctactatctaccacctcagaaagaagctcgttgattctactgataaggctgatctcaggctcatctacctcgctctcgctcacatgatcaagttcagaggacacttcctcatcgagggtgatctcaaccctgataactctgatgtggataagttgttcatccagctcgtgcagacctacaaccagcttttcgaagagaaccctatcaacgcttcaggtgtggatgctaaggctatcctctctgctaggctctctaagtcaagaaggcttgagaacctcattgctcagctccctggtgagaagaagaacggacttttcggaaacttgatcgctctctctctcggactcacccctaacttcaagtctaacttcgatctcgctgaggatgcaaagctccagctctcaaaggatacctacgatgatgatctcgataacctcctcgctcagatcggagatcagtacgctgatttgttcctcgctgctaagaacctctctgatgctatcctcctcagtgatatcctcagagtgaacaccgagatcaccaaggctccactctcagcttctatgatcaagagatacgatgagcaccaccaggatctcacacttctcaaggctcttgttagacagcagctcccagagaagtacaaagagattttcttcgatcagtctaagaacggatacgctggttacatcgatggtggtgcatctcaagaagagttctacaagttcatcaagcctatcctcgagaagatggatggaaccgaggaactcctcgtgaagctcaatagagaggatcttctcagaaagcagaggaccttcgataacggatctatccctcatcagatccacctcggagagttgcacgctatccttagaaggcaagaggatttctacccattcctcaaggataacagggaaaagattgagaagattctcaccttcagaatcccttactacgtgggacctctcgctagaggaaactcaagattcgcttggatgaccagaaagtctgaggaaaccatcaccccttggaacttcgaagaggtggtggataagggtgctagtgctcagtctttcatcgagaggatgaccaacttcgataagaaccttccaaacgagaaggtgctccctaagcactctttgctctacgagtacttcaccgtgtacaacgagttgaccaaggttaagtacgtgaccgagggaatgaggaagcctgcttttttgtcaggtgagcaaaagaaggctatcgttgatctcttgttcaagaccaacagaaaggtgaccgtgaagcagctcaaagaggattacttcaagaaaatcgagtgcttcgattcagttgagatttctggtgttgaggataggttcaacgcatctctcggaacctaccacgatctcctcaagatcattaaggataaggatttcttggataacgaggaaaacgaggatatcttggaggatatcgttcttaccctcaccctctttgaagatagagagatgattgaagaaaggctcaagacctacgctcatctcttcgatgataaggtgatgaagcagttgaagagaagaagatacactggttggggaaggctctcaagaaagctcattaacggaatcagggataagcagtctggaaagacaatccttgatttcctcaagtctgatggattcgctaacagaaacttcatgcagctcatccacgatgattctctcacctttaaagaggatatccagaaggctcaggtttcaggacagggtgatagtctccatgagcatatcgctaacctcgctggatctcctgcaatcaagaagggaatcctccagactgtgaaggttgtggatgagttggtgaaggtgatgggaaggcataagcctgagaacatcgtgatcgaaatggctagagagaaccagaccactcagaagggacagaagaactctagggaaaggatgaagaggatcgaggaaggtatcaaagagcttggatctcagatcctcaaagagcaccctgttgagaacactcagctccagaatgagaagctctacctctactacctccagaacggaagggatatgtatgtggatcaagagttggatatcaacaggctctctgattacgatgttgatgctatcgtgccacagtcattcttgaaggatgattctatcgataacaaggtgctcaccaggtctgataagaacaggggtaagagtgataacgtgccaagtgaagaggttgtgaagaaaatgaagaactattggaggcagctcctcaacgctaagctcatcactcagagaaagttcgataacttgactaaggctgagaggggaggactctctgaattggataaggcaggattcatcaagaggcagcttgtggaaaccaggcagatcactaagcacgttgcacagatcctcgattctaggatgaacaccaagtacgatgagaacgataagttgatcagggaagtgaaggttatcaccctcaagtcaaagctcgtgtctgatttcagaaaggatttccaattctacaaggtgagggaaatcaacaactaccaccacgctcacgatgcttaccttaacgctgttgttggaaccgctctcatcaagaagtatcctaagctcgagtcagagttcgtgtacggtgattacaaggtgtacgatgtgaggaagatgatcgctaagtctgagcaagagatcggaaaggctaccgctaagtatttcttctactctaacatcatgaatttcttcaagaccgagattaccctcgctaacggtgagatcagaaagaggccactcatcgagacaaacggtgaaacaggtgagatcgtgtgggataagggaagggatttcgctaccgttagaaaggtgctctctatgccacaggtgaacatcgttaagaaaaccgaggtgcagaccggtggattctctaaagagtctatcctccctaagaggaactctgataagctcattgctaggaagaaggattgggaccctaagaaatacggtggtttcgattctcctaccgtggcttactctgttctcgttgtggctaaggttgagaagggaaagagtaagaagctcaagtctgttaaggaacttctcggaatcactatcatggaaaggtcatctttcgagaagaacccaatcgatttcctcgaggctaagggatacaaagaggttaagaaggatctcatcatcaagctcccaaagtactcactcttcgaactcgagaacggtagaaagaggatgctcgcttctgctggtgagcttcaaaagggaaacgagcttgctctcccatctaagtacgttaactttctttacctcgcttctcactacgagaagttgaagggatctccagaagataacgagcagaagcaacttttcgttgagcagcacaagcactacttggatgagatcatcgagcagatctctgagttctctaaaagggtgatcctcgctgatgcaaacctcgataaggtgttgtctgcttacaacaagcacagagataagcctatcagggaacaggcagagaacatcatccatctcttcacccttaccaacctcggtgctcctgctgctttcaagtacttcgatacaaccatcgataggaagagatacacctctaccaaagaagtgctcgatgctaccctcatccatcagtctatcactggactctacgagactaggatcgatctctcacagctcggtggtgattcaagggctgatcctaagaagaagaggaaggttggcggaccgggtggatctggaggtggaggttctggaggagaagaacttttgagcaagaattatcatcttgagaacgaagtggctcgtcttaagaaaggttctggcagtggagaagaactgctttcaaagaattaccacctggaaaatgaggtagctagactgaaaaaggggagcggaagtggggaggagttgctgagcaaaaattatcatttggagaacgaagtagcacgactaaagaaagggtccggatcgggtgaggagttactctcgaaaaattatcatctcgaaaacgaagtggctcggctaaaaaagggcagtggttctggagaagagctattatctaaaaactaccacctcgaaaatgaggtggcacgcttaaaaaagggaagtggcagtggtgaagagctactatccaagaattatcatcttgagaacgaggtagcgcgtttgaagaagggttccggctcaggagaggaactgctctcgaagaactatcatcttgaaaatgaggtcgctcgattaaaaaagggatcgggcagtggtgaggaactactttcaaagaattaccacctcgaaaacgaagtagctcgattaaagaaaggttcagggtcgggtgaagaattactgagtaaaaattatcatctggaaaatgaggtagcgagactaaaaaaggggagtggttctggcgaagagttgctatcgaaaaattatcatcttgagaacgaagttgctaggctcaaaaagggctcaggctcaggcgaggagttgctctcgaaaaactaccacttggaaaatgaggtcgcgaggttgaaaaaggggagcgggtcgggcgaggagttattgagcaaaaactatcatttagagaacgaagtcgcgcgcttaaagaaaggctcgggctcgggcgaagaactcttatcgaagaactaccacctcgaaaatgaggtcgccaggttgaaaaagggcagtggcagcggggaggaactcttgagcaagaactaccacttggagaatgaggtcgcgagattgaagaaagggtcggggagcggcgaggaattgctcagcaagaattatcatttggagaacgaagtcgccaggctcaagaaaggctcggggtcgggggaggaattgttgagtaaaaactaccacttggaaaatgaagtcgccaggctcaaaaaagggagtgggagcggcgaagagttattgagcaaaaattaccacttggagaacgaagtggcaaggctcaagaaagggagcggcagcggggaggagctcttatcgaagaactaccacttagagaatgaagtcgcccgcttgaagaaaggctcggggagcggggaagagctcttgagcaagaactaccacttggaaaatgaggtggcgcgcttgaagaaagggagcgggagcggggaagagttactatctaagaattatcatctcgagaacgaggtggctcgactaaagaagggctccggcagtggggaggaactcctgtcgaagaactatcatcttgaaaatgaggttgcaagacttaaaaaggggtccggatcaggtgaggaactactcagtaagaattaccacctggaaaacgaagttgcacgtttgaagaaaggatcaggatcaggcgaagaactgctctcaaaagattatcatttggaaaatgaggttgcacgtttaaaaaagggaagtggcagtggtgaggaacttctgtcgaaaaattatcatctcgagaatgaagtagcccgacttaaaaagggttctggctcgggtggaggcccaaagaagaagaggaaagtctgacgtcgacgatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagccagagctctggtgacggacgtgggcaggtgtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtag

SEQ ID NO: 63 - DNA, 4883 bp, synthetic construct
  source 1..4883: mol_type = other DNA
gagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccacctgcaacgtcccggcgcgccagatttgccttttcaatttcagaaagaatgctaacccacagatggttagagaggcttacgcagcaggtatcatcaagacgatctacccgagcaataatctccaggaaatcaaataccttcccaagaaggttaaagatgcagtcaaaagattcaggactaactgcatcaagaacacagagaaagatatatttctcaagatcagaagtactattccagtatggacgattcaaggcttgcttcacaaaccaaggcaagtaatagagattggagtctctaaaaaggtagttcccactgaatcaaaggccatggagtcaaagattcaaatagaggacctaacagaactcgccgtaaagactggcgaacagttcatacagagtctcttacgactcaatgacaagaagaaaatcttcgtcaacatggtggagcacgacacacttgtctactccaaaaatatcaaagatacagtctcagaagaccaaagggcaattgagacttttcaacaaagggtaatatccggaaacctcctcggattccattgcccagctatctgtcactttattgtgaagatagtggaaaaggaaggtggctcctacaaatgccatcattgcgataaaggaaaggccatcgttgaagatgcctctgccgacagtggtcccaaagatggacccccacccacgaggagcatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggagagaacacgggggactcctgcaggtagatcgctcgtcgacatgccaaagaagaagaggaaagtcatgggccccgacatcgtgatgacccagagccccagcagcctgagcgccagcgtgggcgaccgcgtgaccatcacctgccgcagcagcaccggcgccgtgaccaccagcaactacgccagctgggtgcaggagaagcccggcaagctgttcaagggcctgatcggcggcaccaacaaccgcgcccccggcgtgcccagccgcttcagcggcagcctgatcggcgacaaggccaccctgaccatcagcagcctgcagcccgaggacttcgccacctacttctgcgccctgtggtacagcaaccactgggtgttcggccagggcaccaaggtggagctgaagcgcggcggcggcggcagcggcggcggcggcagcggcggcggcggcagcagcggcggcggcagcgaggtgaagctgctggagagcggcggcggcctggtgcagcccggcggcagcctgaagctgagctgcgccgtgagcggcttcagcctgaccgactacggcgtgaactgggtgcgccaggcccccggccgcggcctggagtggatcggcgtgatctggggcgacggcatcaccgactacaacagcgccctgaaggaccgcttcatcatcagcaaggacaacggcaagaacaccgtgtacctgcagatgagcaaggtgcgcagcgacgacaccgccctgtactactgcgtgaccggcctgttcgactactggggccagggcaccctggtgaccgtgagcagctacccatacgatgttccagattacgctggtggaggcggaggttctgggggaggaggtagtggcggtggtggttcaggaggcggcggaagcttggatccaggtggaggtggaggtagcatggaggccagctctggttcatctcctccccattcacaagagaatcccccagagcacggtggtgatatgggtggtgctccagccgaagaaatcggaggtgaggcagccgatgatttcatgtttgctgaggatacgtttccgtcacttccggattttccgtgtctttccagcccctcctcatctaccttcagctctaactcgtcatccaattcttcttctgcttatactaatacagctggacgcgctggtggcgaaccatccgagcccgcctcagcgggagagggatttgatgcgttggacgacatcgaccaattactcgactttgcttcacttagcatgccctgggatagcgagccttttggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagctcgagccctagacttgtccatcttctggattggccaagttaattaatgtatgaaataaaaggatgcacacatagtgacatgctaatcactataatgtgggcatcaaagttgtgtgttatgtgtaattactaattatctgaataagagaaagagatcatccatatttcttatcctaaatgaatgtcacgtgtctttataattctttgatgaaccagatgcattttattaaccaattccatatacatataaatattaatcatatataattaatatcaattgggttagcaaaacaaatctagtctaggtgtgttttgctaattattgggggatagtgcaaaaagaaatctacgttctcaataattcagatagaaaacttaataaagtgagataatttacatagattgcttttatcctttgatatatgtgaaaccatgcatgatataaggaaaatagatagagaaataattttttacatcgttgaatatgtaaacaatttaattcaagaagctaggaatataaatattgaggagtttatgattagagctcagtgactagcaggtgatgaaaagccttagggtcaggtggcacttttcggggaaatgtgcgatgaacccctatttgtttatttttctaaatacattcaaatatgtatccgctaatgagacaataaccctgataaatgcttcaattatattgaaaaaggaagagtatgagtatccaacatttccgtgtcgcccttattcccttttttgcggccttctgccttcctgtttttgctcacccagaaacgctggtgaaagtaaaagatgctgaagatcagttgggtgcccgagtgggttacattgaactggatctcaacagcggcaaaatcttagagagttttcgccccgaagaacgcttcccaatgatgagcactttcaaagttctgctatgtggcgcggtgttatcccgtattgatgctgggcaagagcaactcggtcgccgcatacactattctcagaatgacttggttgagtattcaccagtcacagaaaagcatcttacggatggcatgacagtaagagaattatgtagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaactctccgcaaactattaactggcgaactacttactcttgcttcacgccagcaactcattgactggatggaggcggacaaagttgcaggaccacttctgcgctcggcacttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagacgaagggaaataaatagtagcccgcctgatgtgcgggcttttttttggtggtggttaccagtgtcgcttagctaccgtaggtcctcttaggcggtccgattagcgacatagtcatcaatgccttgacggcaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctactaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgctcacagcccagcttggagcgaacgacctacaccgaactgagatacctacagcgtgagctatgagaaagcgccacgcttcccgaagggagaaaggcggacaggtatccggtaagcggcagggtcggaacagga

SEQ ID NO: 64 - DNA, 19 bp, synthetic construct
  source 1..19: mol_type = other DNA
aacgtggccaaaatgatgc

SEQ ID NO: 65 - DNA, 20 bp, synthetic construct
  source 1..20: mol_type = other DNA
aaccgcttggtcgactatcg

SEQ ID NO: 66 - DNA, 21 bp, synthetic construct
  source 1..21: mol_type = other DNA
cagcttcaataacgggaattt

SEQ ID NO: 67 - DNA, 20 bp, synthetic construct
  source 1..20: mol_type = other DNA
gttgtaaggtgcagatgagt

SEQ ID NO: 68 - DNA, 20 bp, synthetic construct
  source 1..20: mol_type = other DNA
cagagaaacaatggaacgtg

SEQ ID NO: 69 - DNA, 20 bp, synthetic construct
  source 1..20: mol_type = other DNA
tgagacggtaaggttttacg

SEQ ID NO: 70 - DNA, 29 bp, synthetic construct
  source 1..29: mol_type = other DNA
gttcaaggactttccaaccgcaagatgat

SEQ ID NO: 71 - DNA, 29 bp, synthetic construct
  source 1..29: mol_type = other DNA
ccttctctgcttctccatttgctccaacc

SEQ ID NO: 72 - DNA, 21 bp, synthetic construct
  source 1..21: mol_type = other DNA
ccctgctacaactggaacaac

SEQ ID NO: 73 - DNA, 19 bp, synthetic construct
  source 1..19: mol_type = other DNA
caccctggtgcatacactg

SEQ ID NO: 74 - DNA, 21 bp, synthetic construct
  source 1..21: mol_type = other DNA
agtatggagaaggcaaatggc

SEQ ID NO: 75 - DNA, 23 bp, synthetic construct
  source 1..23: mol_type = other DNA
cacctattccctagaagcctatg

SEQ ID NO: 76 - protein, 64 aa, Zea mays
QPGTEDAEAVALGLGLSDFPSAGKAVLDDEDSFVWPAASFDMGACWAGAGFADPDPACIFLNLP

SEQ ID NO: 77 - protein, 118 aa, Arabidopsis thaliana
GRSACLNFADSAWRLRIPESTCAKDIQKAAAEAALAFQDEMCDATTDHGFDMEETLVEAIYTAEQSENAFYMHDEAMFEMPSLLANMAEGMLLPLPSVQWNHNHEVDGDDDDVSLWSY

SEQ ID NO: 78 - protein, 81 aa, Arabidopsis thaliana
SSDMFDVDELLRDLNGDDVFAGLNQDRYPGNSVANGSYRPESQQSGFDPLQSLNYGIPPFQLEGKDGNGFFDDLSYLDLEN

SEQ ID NO: 79 - protein, 180 aa, synthetic construct
AATNLKGIGPQTIPDMGIMKEFGSPLGFSFQDLDLYGGGGGDQLELQQNMEKPPLDGFMPMNHENLCLGKKRPNPYSGNNGKSPLMWSDDLRLQDLGSCLQDDPFKGDHHHQIQIAPPSLDRGTEMDPMSEIYDSKPEEKKFDASMKLERPSPRRAPLGERMSPMITTGTMAQGRSSPFG

SEQ ID NO: 80 - protein, 65 aa, unidentified
EPEPVRVTAKRRASPEPASSSGNGSMKRRRKAVQKCDGEMASRSSVMQVGCQIEQLTGVHQLLVI

SEQ ID NO: 81 - protein, 351 aa, Arabidopsis thaliana
VSLWEIEPLTTFPMYPSLFPLRLKRPWHAGTSSLPDGRGDLGSGLTWLRGGGGEQQGLLPLNYPSVGLFPWMQQRLDLSQMGTDNNQQYQAMLAAGLQNIGGGDPLRQQFVQLQEPHHQYLQQSASHNSDLMLQQQQQQQASRHLMHAQTQIMSENLPQQNMRQEVSNQPAGQQQQLQQPDQNAYLNAFKMQNGHLQQWQQQSEMPSPSFMKSDFTDSSNKFATTASPASGDGNLLNFSITGQSVLPEQLTTEGWSPKASNTFSEPLSLPQAYPGKSLALEPGNPQNPSLFGVDPDSGLFLPSTVPRFASSSGDAEASPMSLTDSGFQNSLYSCMQDTTHELLHGAGQINS

SEQ ID NO: 82 - protein, 143 aa, synthetic construct
WEKMQQGKEVKEEASDMVTSQSHSHTHSWGETRTPESEIVDNDPFPELDSFPAFQPAPPPATAMMVPKKESMDDATAAAAAAATIPRNNSSLFVDLSYDDIQGMYSGLDMLPPGDDFYSSLFASPRVKGTTPRAGAGMGMVPF

SEQ ID NO: 83 - protein, 183 aa, synthetic construct
IDVAAAGHGDGDMMDQQHATEWTFERLLEEEALTTSTPPPVVVVPNSCCSGALNADRPPVMEEAVTMAPAAVSSAVVGDPMEYNAILRRKLEEDLEAFKMWRADSSVVTSDQRSQGSNNHTGGSSIRNNPVQNKLMNGEDPINNNHAQTAGLGVRLATSSSSRDPSPSDEDMDGEVEILGFKM

SEQ ID NO: 84 - protein, 43 aa, synthetic construct
TVMWEQDAAPFAGAADDFPAFNEEELAWLMELLPQSGSVGGTI

SEQ ID NO: 85 - protein, 47 aa, synthetic construct
PLHQHQDIPLTNQVQPQPEESIGSPQGISQLPSMDDIDDSFWENLFK

SEQ ID NO: 86 - protein, 386 aa, Arabidopsis thaliana
QHQGNMNHSFMTGQDQSFGPLSSLNGFDLQSLAVTGQLPPQSLAQLQAAGLGRPTLAKPGMSVSPLVDQRSIFNFENPKIRFGDGHGQTMNNGNLLHGVPTGSHMRLRPGQNVQSSGMMLPVADQLPRGGPSMLPSLGQQPILSSSVSRRSDLTGALAVRNSIPETNSRVLPTTHSVFNNFPADLPRSSFPLASAPGISVPVSVSYQEEVNSSDAKGGSSAATAGFGNPSYDIFNDFPQHQQHNKNISNKLNDWDLRNMGLVFSSNQDAATATATAAFSTSEAYSSSSTQRKRRETDATVVGEHGQNLQSPSRNLYHLNHVFMDGGSVRVKSERVAETVTCPPANTLFHEQYNQEDLMSAFLKQEGIPSVDNEFEFDGYSIDNIQV

SEQ ID NO: 87 - protein, 53 aa, synthetic construct
EILSLPEDLDLCAAGGGDGPSLSDENDEELFSMFLDVEKLNSTCGASSEAEAE

SEQ ID NO: 88 - protein, 98 aa, synthetic construct
GGMMMNFGGMPHQFGGNQQMFQNNQAMQSMLAAHQLQQLQLHPQAQQQQVLHPQHQQQQPLHPLQAQQLQQAARDLKMKSPMGGQSQWGDGKSGSSGN

SEQ ID NO: 89 - protein, 218 aa, Oryza sativa
PVTESLLELVLRNRLQEWLVEMVMEGHKSTGRDDLGQGAIHLCSFLGYTWAIRLFSLSGFSLDFRDSSGWTALHWAAYHGRERMVATLLSAGANPSLVTDPTPESPAGLTAADLAARQGYDGLAAYLAEKGLTAHFEAMSLSKDTEQSPSKTRLTKLQSEKFEHLSEQELCLKESLAAYRNAADAASNIQAALRERTLKLQTKAIQLANPEIEASEIV

SEQ ID NO: 90 - protein, 64 aa, synthetic construct
HNMPPPLPPQQHQIQHPYMLSHQPSPFLNMGGLYQEDDPMAMRRNDLELTLEPVYNCNLGCFAA

SEQ ID NO: 91 - protein, 41 aa, synthetic construct
GGVAPSDDAVDEEVTDTEWFFLVSMTQSFACGAGLAGKAFA

SEQ ID NO: 92 - protein, 120 aa, Zea mays
MEASSGSSPPHSQENPPEHGGDMGGAPAEEIGGEAADDFMFAEDTFPSLPDFPCLSSPSSSTFSSNSSSNSSSAYTNTAGRAGGEPSEPASAGEGFDALDDIDQLLDFASLSMPWDSEPF

SEQ ID NO: 93 - protein, 176 aa, Oryza sativa
PPPSYYSMDHKEYAYGHATKEVHGEHAFFSDGTEREHHHAAAGHGQWQFKQLGMEPKQSTTPLFPGAGYGHTAASPYAIDLSKEDDDEKERRQQQQQQQQHCFLLGADLRLEKPAGHDHAAAAQKPLRHFFDEWPHEKNSKGSWMGLEGETQLSMSIPMAANDLPITTTSRYHNDE

SEQ ID NO: 94 - protein, 104 aa, Arabidopsis thaliana
KEKKSLFGLDVGRKRRLTSTPSLGTMEENLLHDQEFDRMKDDMEMLFAAAIDDEANNSMPTKEEQCLEAMNVMMRDGNLEAALDVKVEDLVGSPLDWDSQDLHD

SEQ ID NO: 95 - protein, 146 aa, Arabidopsis thaliana
KEKRKEIEEAISKKRQRPIDQGKRNVEDYGDESGYGNDVAASSSALIGMSQEYTYGNMSEFEMSELDKLAMHIQGLGDNSSAREEVLNVEKGNDEEEVEDQQQGYHKENNEIYGEGFWEDLLNEGQNFDFEGDQENVDVGSSSHTN

SEQ ID NO: 96 - protein, 418 aa, synthetic construct
KTTGALLDADIGKTSATYGTISSDVTHGEMVVDVTIEDSYSTESAWVRCDDCFKWRRIPASVVGSIDESSRWICMNNSDKRFADCSKSQEMSNEEINEELGIGQDEADAYDCDAAKRGKEKEQKSKRLTGKQKACFKAIKTNQFLHRNRKSQTIDEIMVCHCKPSPDGRLGCGEECLNRMLNIECLQGTCPAGDLCSNQQFQKRKYVKFERFQSGKKGYGLRLLEDVREGQFLIEYVGEVLDMQSYETRQKEYAFKGQKHFYFMTLNGNEVIDAGAKGNLGRFINHSCEPNCRTEKWMVNGEICVGIFSMQDLKKGQELTFDYNYVRVFGAAAKKCYCGSSHCRGYIGGDPLNGDVIIQSDSDEEYPELVILDDDESGEGILGATSRTFTDDADEQMPQSFEKVNGYKDLAPDNTQTQ

SEQ ID NO: 97 - protein, 248 aa, synthetic construct
MAATLGRDQYVYMAKLAEQAERYEEMVQFMEQLVTGATPAEELTVEERNLLSVAYKNVIGSLRAAWRIVSSIEQKEESRKNDEHVSLVKDYRSKVESELSSVCSGILKLLDSHLIPSAGASESKVFYLKMKGDYHRYMAEFKSGDERKTAAEDTMLAYKAAQDIAAADMAPTHPIRLGLALNFSVFYYEILNSSDKACNMAKQAFEEAIAELDTLGEESYKDSTLIMQLLRDNLTLWTSDMQEQMDEA

SEQ ID NO: 98 - protein, 318 aa, synthetic construct
SLSGGSCSLLMNDCSQYDVEGFEKESHYEVEELKPEKVMNSSNFGMVAKMHDFPVKEEVPAGNSEFMRKRKPNRDLNTIMDRTVFTCENLGCAHSEISRGFLDRNSRDNHQLACPHRDSRLPYGAAPSRFHVNEVKPVVGFPQPRPVNSVAQPIDLTGIVPEDGQKMISELMSMYDRNVQSNQTSMVMENQSVSLLQPTVHNHQEHLQFPGNMVEGSFFEDLNIPNRANNNNSSNNQTFFQGNNNNNNVFKFDTADHNNFEAAHNNNNNSSGNRFQLVFDSTPFDMASFDYRDDMSMPGVVGTMDGMQQKQQDVSIWF

SEQ ID NO: 99 - protein, 106 aa, synthetic construct
MNDADTNLGSSFSDDTHSVFEFPELDLSDEWMDDDLVSAVSGMNQSYGYQTSDVAGALFSGSSSCFSHPESPSTKTYVAATATASADNQNKKEKKKIKGRVAFKTR

SEQ ID NO: 100 - protein, 173 aa, synthetic construct
DGADVLKMSQLKARKKHLRFQQSKIHDWGLVALEPIEAEDFVIEYVGELIRSSISEIRERQYEKMGIGSSYLFRLDDGYVLDATKRGGIARFINHSCEPNCYTKIISVEGKKKIFIYAKRHIDAGEEISYNYKFPLEDDKIPCNCGAPNVYCFCEQVPWIAKLKRRTWFSRRN

SEQ ID NO: 101 - protein, 196 aa, synthetic construct
MSAIKITNVAVLHNPAPFVSPFQFEISYECLNSLKDDLEWKLIYVGSAEDETYDQLLESVLVGPVNVGNYRFVFQADPPDPSKIQEEDIIGVTVLLLTCSYMGQEFLRVGYYVNNDYEDEQLKEEPPTKVLIDKVQRNILSDKPRVTKFPIDFHPEEEQTAATAAPPEQSDEQQPNVNGEAQVLPDQSVEPKPEES

SEQ ID NO: 102 - protein, 418 aa, synthetic construct
LESSDGGKDGGSSVVGTGVSGTVGGSSISGLVPKDESVKVLAENFQTSGAYIAREEALKREEQAGRLKFVCYSNDSIDEHMMCLIGLKNIFARQLPNMPKEYIVRLLMDRKHKSVMVLRGNLVVGGITYRPYHSQKFGEIAFCAITADEQVKGYGTRLMNHLKQHARDVDGLTHFLTYADNNAVGYFVKQGFTKEIYLEKDVWHGFIKDYDGGLLMECKIDPKLPYTDLSSMIRQQRKAIDERIRELSNCQNVYPKIEFLKNEAGIPRKIIKVEEIRGLREAGWTPDQWGHTRFKLFNGSADMVTNQKQLNALMRALLKTMQDHADAWPFKEPVDSRDVPDYYDIIKDPIDLKVIAKRVESEQYYVTLDMFVADARRMFNNCRTYNSPDTIYYKCATRLETHFHSKVQAGLQSGAKSQ

SEQ ID NO: 103 - protein, 214 aa, synthetic construct
RYKACETTEPDAIRQQFPFYNEIQSIFEARMQRMLWSEATEPSTSSKRKHHQFSSDDEEEEVDEPNQDINEELLSLVETQKRETEVITTSTSTNPRKRAKKGKGVASGTKAETAGNTLKDILEEFMRQTVKMEKEWRDAWEMKEIEREKREKEWRRRMAELEEERAATERRWMEREEERRLREEARAQKRDSLIDALLNRLNRDHNDDHHNQGF

SEQ ID NO: 104 - protein, 50 aa, synthetic construct
LCVDIYKRAFPRSVNKGRSSYAGGSGGVPGISYNSKVRDYILWGVTEVIP

SEQ ID NO: 105 - protein, 218 aa, synthetic construct
MSSINITNVTVLDNPAPFVNPFQFEISYECLTSLKDDLEWKLIYVGSAEDETYDQVLESVLVGPVNVGNYRFVLQADSPDPLKIREEDIIGVTVLLLTCSYMDQEFIRVGYYVNNDYDDEQLREEPPTKVLIDKVQRNILTDKPRVTKFPINFHPENEQTLGDGPAPTEPFADSVVNGEAPVFLEQPQKLQEIEQFDDSDVNGEAIALLDQPQNLQET

SEQ ID NO: 106 - protein, 200 aa, synthetic construct
MTDQGLEGSNPVDLSKHPSGIVPTLQNIVSTVNLDCKLDLKAIALQARNAEYNPKRFAAVIMRIREPKTTALIFASGKMVCTGAKSEDFSKMAARKYARIVQKLGFPAKFKDFKIQNIVGSCDVKFPIRLEGLAYSHAAFSSYEPELFPGLIYRMKVPKIVLLIFVSGKIVITGAKMRDETYKAFENIYPVLSEFRKIQQ

SEQ ID NO: 107 - protein, 188 aa, synthetic construct
DIQQGSAYASFLSKKSSRIEDQLRFCTDQFQKLAEDKYQKSVSLENLQKKRADIGNGLEQARSRLEESHSKVEQSRLDYGALELELEIERFNRRRIEEEMEIAKKKVSRLRSLIEGSSAIQKLRQELSEFKEILKCKACNDRPKEVVITKCYHLFCNPCVQKLTGTRQKKCPTCSASFGPNDIKPIYI

SEQ ID NO: 108 - protein, 300 aa, synthetic construct
MESEFQQHHFLLHDHQHQRPRNSGLIRYQSAPSSYFSSFGESIEEFLDRPTSPETERILSGFLQTTDTSDNVDSFLHHTFNSDGTEKKPPEVKTEDEDAEIPVTATATAMEVVVSGDGEISVNPEVSIGYVASVSRNKRPREKDDRTPVNNLARHNSSPAGLFSSIDVETAYAAVMKSMGGFGGSNVMSTSNTEASSLTPRSKLLPPTSRAMSPISEVDVKPGFSSRLPPRTLSGGFNRSFGNEGSASSKLTALARTQSGGLDQYKTKDEDSASRRPPLAHHMSLPKSLSDIEQLLSDSI

SEQ ID NO: 109 - protein, 105 aa, synthetic construct
AKQLEQLYDSLRQEYDVVSREKQMLHDEVKKLRALLRDQGLIKKQISAGTIKVSGEEDTVEISSVVVAHPRTENMNANQITGGNQVYGQYNNPMLVASSGWPSYP

SEQ ID NO: 110 - protein, 267 aa, synthetic construct
CCANCKGNCLSADFPCTCARETSGEYAYTKEGLLKEKFLDTCLKMKKEPDSFPKVYCKDCPLERDHDKGTYGKCDGHLIRKFIKECWRKCGCDMQCGNRVVQRGIRCQLQVYFTQEGKGWGLRTLQDLPKGTFICEYIGEILTNTELYDRNVRSSSERHTYPVTLDADWGSEKDLKDEEALCLDATICGNVARFINHRCEDANMIDIPIEIETPDRHYYHIAFFTLRDVKAMDELTWDYMIDFNDKSHPVKAFRCCCGSESCRDRKI

SEQ ID NO: 111 - protein, 140 aa, synthetic construct
EINYHSTGLMDSAHNHFKRARLFEDLEDEDAEVIFPSSVYPSPLPESTVPANKGYASSAIQTLFTGPVKAEEPTPTPKIPKKRGRKKKNADPEEINSSAPRDDDPENRSKFYESASARKRTVTAEERERAINAAKTFEPT

SEQ ID NO: 112 - protein, 144 aa, synthetic construct
RQFDTLKAENDLLQTHNQKLQAEIMGLKNREQTESINLNKETEGSCSNRSDNSSDNLRLDISTAPPSNDSTLTGGHPPPPQTVGRHFFPPSPATATTTTTTMQFFQNSSSGQSMVKEENSISNMFCAMDDHSGFWPWLDQQQYN

SEQ ID NO: 113 - protein, 340 aa, synthetic construct
MVTRETKLTSEREVESSMAQARHNGGGGGENHPFTSLGRQSSIYSLTLDEFQHALCENGKNFGSMNMDEFLVSIWNAEENNNNQQQAAAAAGSHSVPANHNGFNNNNNNGGEGGVGVFSGGSRGNEDANNKRGIANESSLPRQGSLTLPAPLCRKTVDEVWSEIHRGGGSGNGGDSNGRSSSSNGQNNAQNGGETAARQPTFGEMTLEDFLVKAGVVREHPTNPKPNPNPNQNQNPSSVIPAAAQQQLYGVFQGTGDPSFPGQAMGVGDPSGYAKRTGGGGYQQAPPVQAGVCYGGGVGFGAGGQQMGMVGPLSPVSSDGLGHGQVDNIGGQYGVDMGGL

SEQ ID NO: 114 - protein, 162 aa, synthetic construct
FPARKTKEFQAVHKVFGVSNVQKMVRTVREEDRTKLSDSLTWEALWRQKDPVLGSYGEYRRICEELKLYKSLVHNQPLIGWDNNQRVFNNNSNNKNGLAMTNSSGSGGFSVNNNGVGVNREIVNGGYASRNVQGGWENLKHDQRQQCYAVINNGFKQHYLPL

SEQ ID NO: 115 - protein, 221 aa, synthetic construct
SSVYKPTSKSSSSSSSNQDFSVPGSSISNPPPLKKPLSAPPQSNKIQPFSKINGGDASVNQWGSTSTISEYLMDTLPGWHVEDFLDSSLPTYGFSKSGDDDGVLPYMEPEDDNNTKRNNNNNNNNNNNTVSLPSKNLGIWVPQIPQTLPSSYPNQYFSQDNNIQFGMYNKETSPEVVSFAPIQNMKQQGQNNKRWYDDGGFTVPQITPPPLSSNKKFRSFW

SEQ ID NO: 116 - DNA, 23294 bp, synthetic construct
  source 1..23294: mol_type = other DNA
tggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgaacatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggaagatgctccaaagaagaagagaaaggtcgacggcattggtagtgggagcaacggcagcagcggatcccttaagcttgccaccatgcgtacgggcaagaatgagcaggaactgctggaactggacaaatgggcaagcctggggagcggctccggaaagaacgagcaggaactgctggaactggacaagtgggcttctctgggctccgggtccgggaaaaacgaacaggagctgctggagctggataagtgggccagcctgggctccgggtccgggaagaatgaacaggaactgctggaactggataaatgggcatctctgggcagcgggtccggaaagaatgaacaggagctgctggaactggacaagtgggcatccctggggagcgggtccggaaagaatgaacaggaactgctggaactggataagtgggcatccctgggaagcggctccggaaaaaacgagcaggagctgctggagctggacaagtgggcctctctgggctccggctccgggaagaatgaacaggagctgctggaactggataagtgggcatctctggggtccggaagcggtaaaaacgagcaggaactgctggaactggacaagtgggcaagcctggggtctggctccggcaaaaacgaacaggaactgctggaactggacaaatgggccagcctgggctccgggtccgggaaaaatgagcaggaactgctggaactggataaatgggcgagcctgggcagcgggtccggaaaaaacgaacaggagctgctggagctggacaaatgggcgagtctgggctccggaagcggcaagaatgaacaggaactgctggagctggacaaatgggccagcctgggcagcggctccggaaagaatgagcaggaactgctggagctggacaaatgggcatctctgggctccggctccgggaaaaacgagcaggaactgctggagctggacaagtgggcttccctgggcagcggctccggaaaaaatgaacaggaactgctggaactggacaagtgggccagcctggggtccgggtctgggaagaacgagcaggaactgctggagctggacaagtgggcatctctggggtctggctctggcaagaacgagcaggaactgctggaactggacaagtgggcgagcctgggcagcgggtctggcaagaatgaacaggagctgctggaactggacaaatgggccagcctggggtctgggtctggaaagaatgagcaggaactgctggagctggacaagtgggcatctctgggctctgggtctggcaagaacgagcaggagctgctggagctggataagtgggcatccctggggtccgggtccggcaagaatgaacaggagctgctggagctggataaatgggcatccctgggctctgggtctgggaagaatgagcaggagctgctggaactggataagtgggcgagcctgggcagcgggtctggcaagaatgagcaggaactgctggagctggacaagtgggcttctctgcaccggtcagcagtggaggacagcacgtaacccgggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacggcgcgccggagtggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggatttgcattaactcgggtgttttagagctagaaatagcaagttaaaataaggctagtccgttatcaacttgaaaaagtggcaccgagtcggtgctttttttcccggtgacgcaatggtctattggcgttgttgttttccattagggagcttatagattattgattaatatgatattgtggagccacgagtatcattgataagatataaatttggccaaaaatgcttttgattgaaatatgcaacaccattaagagtgtttttgtgaaaaaggaatgctcgtactgcaagctatggtttcttctattgtatgacattgccattacttgctatctctattgattcattttaagcactttcctgataaagaatctgttttatccggttgattgctcttatttgtctgttactgctcaaaccttacactatactcccaatgtttctatgccacaggttaatctaaagaggtgaccgaaagaagaaaacttttacgttttgcatggagtaagaatgccagagttaagaagtggagcaaggagatcgagacgccttgacgagcagcctaaccctccgctagttgaacaagcagaaaatatcgtacttcctcctcagactgcaacaaggagaagaggtggtggctttggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtccagtgtattagtgtggtgttttagagctagaaatagcaagttaaaataaggctagtccgttatcaacttgaaaaagtggcaccgagtcggtgctttttttcccggtgacgccgctgagctcccggggcgcgccctatggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctctcccggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgccaaagaagaagaggaaagtcatggaggtgcagctggtggaatctgggggcggactggtgcagcccgggggatctctgcggctgtcctgcgccgcctctggctccatctctagcgtggatgtgatgtcctggtacaggcaggcccctggcaagcagagggaactggtggccttcattactgataggggaagaaccaattacaaagtgagcgtgaaaggccgcttcactatcagccgggataattctaagaatatggtgtatctgcagatgaacagcctgaagccagaagacactgccgactatctgtgcagggctgagtcccggacttcctggtccagcccatctcctctggatgtgtgggggcgcggcactcaggtgactgtgtcctccttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtagtggcggtggtggttcaggaggcggcggaagcttagatccaggtggaggtggaggtagcaccgtgatgtgggaacaggatgcagccccctttgcaggcgcagccgatgactttccagcttttaacgaggaggaattagcgtggctgatggagctgctccctcaaagcggatctgtgggggggaccattggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgctgagctcagtg

SEQ ID NO: 117 - DNA, 23357 bp, synthetic construct
  source 1..23357: mol_type = other DNA
tggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgaacatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggaagatgctccaaagaagaagagaaaggtcgacggcattggtagtgggagcaacggcagcagcggatcccttaagcttgccaccatgcgtacgggcaagaatgagcaggaactgctggaactggacaaatgggcaagcctggggagcggctccggaaagaacgagcaggaactgctggaactggacaagtgggcttctctgggctccgggtccgggaaaaacgaacaggagctgctggagctggataagtgggccagcctgggctccgggtccgggaagaatgaacaggaactgctggaactggataaatgggcatctctgggcagcgggtccggaaagaatgaacaggagctgctggaactggacaagtgggcatccctggggagcgggtccggaaagaatgaacaggaactgctggaactggataagtgggcatccctgggaagcggctccggaaaaaacgagcaggagctgctggagctggacaagtgggcctctctgggctccggctccgggaagaatgaacaggagctgctggaactggataagtgggcatctctggggtccggaagcggtaaaaacgagcaggaactgctggaactggacaagtgggcaagcctggggtctggctccggcaaaaacgaacaggaactgctggaactggacaaatgggccagcctgggctccgggtccgggaaaaatgagcaggaactgctggaactggataaatgggcgagcctgggcagcgggtccggaaaaaacgaacaggagctgctggagctggacaaatgggcgagtctgggctccggaagcggcaagaatgaacaggaactgctggagctggacaaatgggccagcctgggcagcggctccggaaagaatgagcaggaactgctggagctggacaaatgggcatctctgggctccggctccgggaaaaacgagcaggaactgctggagctggacaagtgggcttccctgggcagcggctccggaaaaaatgaacaggaactgctggaactggacaagtgggccagcctggggtccgggtctgggaagaacgagcaggaactgctggagctggacaagtgggcatctctggggtctggctctggcaagaacgagcaggaactgctggaactggacaagtgggcgagcctgggcagcgggtctggcaagaatgaacaggagctgctggaactggacaaatgggccagcctggggtctgggtctggaaagaatgagcaggaactgctggagctggacaagtgggcatctctgggctctgggtctggcaagaacgagcaggagctgctggagctggataagtgggcatccctggggtccgggtccggcaagaatgaacaggagctgctggagctggataaatgggcatccctgggctctgggtctgggaagaatgagcaggagctgctggaactggataagtgggcgagcctgggcagcgggtctggcaagaatgagcaggaactgctggagctggacaagtgggcttctctgcaccggtcagcagtggaggacagcacgtaacccgggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacggcgcgccggagtggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggatttgcattaactcgggtgttttagagctagaaatagcaagttaaaataaggctagtccgttatcaacttgaaaaagtggcaccgagtcggtgctttttttcccggtgacgcaatggtctattggcgttgttgttttccattagggagcttatagattattgattaatatgatattgtggagccacgagtatcattgataagatataaatttggccaaaaatgcttttgattgaaatatgcaacaccattaagagtgtttttgtgaaaaaggaatgctcgtactgcaagctatggtttcttctattgtatgacattgccattacttgctatctctattgattcattttaagcactttcctgataaagaatctgttttatccggttgattgctcttatttgtctgttactgctcaaaccttacactatactcccaatgtttctatgccacaggttaatctaaagaggtgaccgaaagaagaaaacttttacgttttgcatggagtaagaatgccagagttaagaagtggagcaaggagatcgagacgccttgacgagcagcctaaccctccgctagttgaacaagcagaaaatatcgtacttcctcctcagactgcaacaaggagaagaggtggtggctttggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtccagtgtattagtgtggtgttttagagctagaaatagcaagttaaaataaggctagtccgttatcaacttgaaaaagtggcaccgagtcggtgctttttttcccggtgacgccgctgagctcccggggcgcgccctatggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctctcccggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgccaaagaagaagaggaaagtcatggaggtgcagctggtggaatctgggggcggactggtgcagcccgggggatctctgcggctgtcctgcgccgcctctggctccatctctagcgtggatgtgatgtcctggtacaggcaggcccctggcaagcagagggaactggtggccttcattactgataggggaagaaccaattacaaagtgagcgtgaaaggccgcttcactatcagccgggataattctaagaatatggtgtatctgcagatgaacagcctgaagccagaagacactgccgactatctgtgcagggctgagtcccggacttcctggtccagcccatctcctctggatgtgtgggggcgcggcactcaggtgactgtgtcctccttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtagtggcggtggtggttcaggaggcggcggaagcttagatccaggtggaggtggaggtagccaaccgggcacagaggacgcagaggccgtagcattagggttgggtctgtcagattttccttcggctggaaaggccgtcctggacgacgaagatagcttcgtctggccagcggcgtcgttcgacatgggggcctgttgggcaggagccggttttgctgatccggatccggcttgcattttcttgaatctacctggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgctgagctcagtg

SEQ ID NO: 118 - DNA, 23408 bp, synthetic construct
  source 1..23408: mol_type = other DNA
tggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatgctagagcagcttgagcttggatcagattgtcgtttcccgccttcagtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttcccctcgggatcaaagtactttgatccaacccctccgctgctatagtgcagtcggcttctgacgttcagtgcagccgtcttctgaaaacgacatgtcgcacaagtcctaagttacgcgacaggctgccgccctgcccttttcctggcgttttcttgtcgcgtgttttagtcgcataaagtagaatacttgcgactagaaccggagacattacgccatgaacaagagcgccgccgctggcctgctgggctatgcccgcgtcagcaccgacgaccaggacttgaccaaccaacgggccgaactgcacgcggccggctgcaccaagctgttttccgagaagatcaccggcaccaggcgcgaccgcccggagctggccaggatgcttgaccacctacgccctggcgacgttgtgacagtgaccaggctagaccgcctggcccgcagcacccgcgacctactggacattgccgagcgcatccaggaggccggcgcgggcctgcgtagcctggcagagccgtgggccgacaccaccacgccggccggccgcatggtgttgaccgtgttcgccggcattgccgagttcgagcgttccctaatcatcgaccgcacccggagcgggcgcgaggccgccaaggcccgaggcgtgaagtttggcccccgccctaccctcaccccggcacagatcgcgcacgcccgcgagctgatcgaccaggaaggccgcaccgtgaaagaggcggctgcactgcttggcgtgcatcgctcgaccctgtaccgcgcacttgagcgcagcgaggaagtgacgcccaccgaggccaggcggcgcggtgccttccgtgaggacgcattgaccgaggccgacgccctggcggccgccgagaatgaacgccaagaggaacaagcatgaaaccgcaccaggacggccaggacgaaccgtttttcattaccgaagagatcgaggcggagatgatcgcggccgggtacgtgttcgagccgcccgcgcacggctcaaccgtgcggctgcatgaaatcctggccggtttgtctgatgccaagctggcggcctggccggccagcttggccgctgaagaaaccgagcgccgccgtctaaaaaggtgatgtgtatttgagtaaaacagcttgcgtcatgcggtcgctgcgtatatgatgcgatgagtaaataaacaaatacgcaaggggaacgcatgaaggttatcgctgtacttaaccagaaaggcgggtcaggcaagacgaccatcgcaacccatctagcccgcgccctgcaactcgccggggccgatgttctgttagtcgattccgatccccagggcagtgcccgcgattgggcggccgtgcgggaagatcaaccgctaaccgttgtcggcatcgaccgcccgacgattgaccgcgacgtgaaggccatcggccggcgcgacttcgtagtgatcgacggagcgccccaggcggcggacttggctgtgtccgcgatcaaggcagccgacttcgtgctgattccggtgcagccaagcccttacgacatatgggccaccgccgacctggtggagctggttaagcagcgcattgaggtcacggatggaaggctacaagcggcctttgtcgtgtcgcgggcgatcaaaggcacgcgcatcggcggtgaggttgccgaggcgctggccgggtacgagctgcccattcttgagtcccgtatcacgcagcgcgtgagctacccaggcactgccgccgccggcacaaccgttcttgaatcagaacccgagggcgacgctgcccgcgaggtccaggcgctggccgctgaaattaaatcaaaactcatttgagttaatgaggtaaagagaaaatgagcaaaagcacaaacacgctaagtgccggccgtccgagcgcacgcagcagcaaggctgcaacgttggccagcctggcagacacgccagccatgaagcgggtcaactttcagttgccggcggaggatcacaccaagctgaagatgtacgcggtacgccaaggcaagaccattaccgagctgctatctgaatacatcgcgcagctaccagagtaaatgagcaaatgaataaatgagtagatgaattttagcggctaaaggaggcggcatggaaaatcaagaacaaccaggcaccgacgccgtggaatgccccatgtgtggaggaacgggcggttggccaggcgtaagcggctgggttgtctgccggccctgcaatggcactggaacccccaagcccgaggaatcggcgtgacggtcgcaaaccatccggcccggtacaaatcggcgcggcgctgggtgatgacctggtggagaagttgaaggccgcgcaggccgcccagcggcaacgcatcgaggcagaagcacgccccggtgaatcgtggcaagcggccgctgatcgaatccgcaaagaatcccggcaaccgccggcagccggtgcgccgtcgattaggaagccgcccaagggcgacgagcaaccagattttttcgttccgatgctctatgacgtgggcacccgcgatagtcgcagcatcatggacgtggccgttttccgtctgtcgaagcgtgaccgacgagctggcgaggtgatccgctacgagcttccagacgggcacgtagaggtttccgcagggccggccggcatggccagtgtgtgggattacgacctggtactgatggcggtttcccatctaaccgaatccatgaaccgataccgggaagggaagggagacaagcccggccgcgtgttccgtccacacgttgcggacgtactcaagttctgccggcgagccgatggcggaaagcagaaagacgacctggtagaaacctgcattcggttaaacaccacgcacgttgccatgcagcgtacgaagaaggccaagaacggccgcctggtgacggtatccgagggtgaagccttgattagccgctacaagatcgtaaagagcgaaaccgggcggccggagtacatcgagatcgagctagctgattggatgtaccgcgagatcacagaaggcaagaacccggacgtgctgacggttcaccccgattactttttgatcgatcccggcatcggccgttttctctaccgcctggcacgccgcgccgcaggcaaggcagaagccagatggttgttcaagacgatctacgaacgcagtggcagcgccggagagttcaagaagttctgtttcaccgtgcgcaagctgatcgggtcaaatgacctgccggagtacgatttgaaggaggaggcggggcaggctggcccgatcctagtcatgcgctaccgcaacctgatcgagggcgaagcatccgccggttcctaatgtacggagcagatgctagggcaaattgccctagcaggggaaaaaggtcgaaaaggcctctttcctgtggatagcacgtacattgggaacccaaagccgtacattgggaaccggaacccgtacattgggaacccaaagccgtacattgggaaccggtcacacatgtaagtgactgatataaaagagaaaaaaggcgatttttccgcctaaaactctttaaaacttattaaaactcttaaaacccgcctggcctgtgcataactgtctggccagcgcacagcccaagagctgcaaaaagcgcctacccttcggtcgctgcgctccctacgccccgccgcttcgcgtcggcctatcgcggccgctggccgctcaaaaatggctggcctacggccaggcaatctaccagggcgcggacaagccgcgccgtcgccactcgaccgccggcgcccacatcaaggcaccctgcctcgcgcgtttcggtgatgacggtgaaaacctctgacacatgcagctcccggaaacggtcacagcttgtctgtaagcggatgccgggagcagacaagcccgtcagggcgcgtcagcgggtgttggcgggtgtcggggcgcagccatgacccagtcacgtagcgatagcggagtgtatactggcttaactatgcggcatcagagcagattgtactgagagtgcaccatatgcggtgtgaaataccgcacagatgcgtaaggagaaaataccgcatcaggccctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgcattctaggtactaaaacaattcatccagtaaaatataatattttattttctcccaatcaggcttgatccccagtaagtcaaaaaatagctcgacatactgttcttccccgatatcctccctgatcgaccggacgcagaaggcaatgtcataccacttgtccgccctgccgcttctcccaagatcaataaagccacttactttgccatctttcacaaagatgttgctgtctcccaggtcgccgtgggaaaagacaagttcctcttcgggcttttccgtctttaaaaaatcatacagctcgcgcggatctttaaatggagtgtcttcttcccagttttcgcaatccacatcggccagatcgttattcagtaagtaatccaattcggctaagcggctgtctaagctattcgtatagggacaatccgatatgtcgatggagtgaaagagcctgatgcactccgcatacagctcgataatcttttcagggctttgttcatcttcatactcttccgagcaaaggacgccatcggcctcactcatgagcagattgctccagccatcatgccgttcaaagtgcaggacctttggaacaggcagctttccttccagccatagcatcatgtccttttcccgttccacatcataggtggtccctttataccggctgtccgtcatttttaaatataggttttcattttctcccaccagcttatataccttagcaggagacattccttccgtatcttttacgcagcggtatttttcgatcagttttttcaattccggtgatattctcattttagccatttattatttccttcctcttttctacagtatttaaagataccccaagaagctaattataacaagacgaactccaattcactgttccttgcattctaaaaccttaaataccagaaaacagctttttcaaagttgttttcaaagttggcgtataacatagtatcgacggagccgattttgaaaccgcggtgatcacaggcagcaacgctctgtcatcgttacaatcaacatgctaccctccgcgagatcatccgtgtttcaaacccggcagcttagttgccgttcttccgaatagcatcggtaacatgagcaaagtctgccgccttacaacggctctcccgctgacgccgtcccggactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactgaattaacgccgaattaattcgggggatctggattttagtactggattttggttttaggaattagaaattttattgatagaagtattttacaaatacaaatacatactaagggtttcttatatgctcaacacatgagcgaaaccctataggaaccctaattcccttatctgggaactactcacacattattatggagaaactcgagcttgtcgatcgactctagctagaggatcgatccgaaccccagagtcccgctcagaagaactcgtcaagaaggcgatagaaggcgatgcgctgcgaatcgggagcggcgataccgtaaagcacgaggaagcggtcagcccattcgccgccaagttcttcagcaatatcacgggtagccaacgctatgtcctgatagcggtccgccacacccagccggccacagtcgatgaatccagaaaagcggccattttccaccatgatattcggcaagcaggcatcgccatgtgtcacgacgagatcctcgccgtcgggcatgcgcgccttgagcctggcgaacagttcggctggcgcgagcccctgatgttcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctggagttcattcagggcaccggacaggtcggtcttgacaaaaagaaccgggcgcccctgcgctgacagccgaaacacggcggcatcagagcagccgattgtctgttgtgcccagtcatagccgaatagcctctccacccaagcggccggagaacctgcgtgcaatccatcttgttcaatccccatggtcgatcgacagatctgcgaaagctcgagagagatagatttgtagagagagactggtgatttcagcgtgtcctctccaaatgaaatgaacttccttatatagaggaagggtcttgcgaaggatagtgggattgtgcgtcatcccttacgtcagtggagatatcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttcacatcaatccacttgctttgaagacgtggttggaacgtcttctttttccacgatgctcctcgtgggtgggggtccatctttgggaccactgtcggcagaggcatcttgaacgatagcctttcctttatcgcaatgatggcatttgtaggtgccaccttccttttctactgtccttttgatgaagtgacagatagctgggcaatggaatccgaggaggtttcccgatattaccctttgttgaaaagtctcaatagccctttggtcttctgagactgtatctttgatattcttggagtagacgagagtgtcgtgctccaccatgttggcaagctgctctagccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgacatgattacgaattcgagctcggtacccggggatcggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgaacatggacaagaagtacagcatcggcctggccatcggcaccaactctgtgggctgggccgtgatcaccgacgagtacaaggtgcccagcaagaaattcaaggtgctgggcaacaccgaccggcacagcatcaagaagaacctgatcggcgccctgctgttcgacagcggagaaacagccgaggccacccggctgaagagaaccgccagaagaagatacaccagacggaagaaccggatctgctatctgcaagagatcttcagcaacgagatggccaaggtggacgacagcttcttccacagactggaagagtccttcctggtggaagaggataagaagcacgagcggcaccccatcttcggcaacatcgtggacgaggtggcctaccacgagaagtaccccaccatctaccacctgagaaagaaactggtggacagcaccgacaaggccgacctgcggctgatctatctggccctggcccacatgatcaagttccggggccacttcctgatcgagggcgacctgaaccccgacaacagcgacgtggacaagctgttcatccagctggtgcagacctacaaccagctgttcgaggaaaaccccatcaacgccagcggcgtggacgccaaggccatcctgtctgccagactgagcaagagcagacggctggaaaatctgatcgcccagctgcccggcgagaagaagaatggcctgttcggcaacctgattgccctgagcctgggcctgacccccaacttcaagagcaacttcgacctggccgaggatgccaaactgcagctgagcaaggacacctacgacgacgacctggacaacctgctggcccagatcggcgaccagtacgccgacctgtttctggccgccaagaacctgtccgacgccatcctgctgagcgacatcctgagagtgaacaccgagatcaccaaggcccccctgagcgcctctatgatcaagagatacgacgagcaccaccaggacctgaccctgctgaaagctctcgtgcggcagcagctgcctgagaagtacaaagagattttcttcgaccagagcaagaacggctacgccggctacatcgatggcggagccagccaggaagagttctacaagttcatcaagcccatcctggaaaagatggacggcaccgaggaactgctcgtgaagctgaacagagaggacctgctgcggaagcagcggaccttcgacaacggcagcatcccccaccagatccacctgggagagctgcacgccattctgcggcggcaggaagatttttacccattcctgaaggacaaccgggaaaagatcgagaagatcctgaccttccgcatcccctactacgtgggccctctggccaggggaaacagcagattcgcctggatgaccagaaagagcgaggaaaccatcaccccctggaacttcgaggaagtggtggacaagggcgccagcgcccagagcttcatcgagcggatgaccaacttcgataagaacctgcccaacgagaaggtgctgcccaagcacagcctgctgtacgagtacttcaccgtgtacaacgagctgaccaaagtgaaatacgtgaccgagggaatgagaaagcccgccttcctgagcggcgagcagaaaaaagccatcgtggacctgctgttcaagaccaaccggaaagtgaccgtgaagcagctgaaagaggactacttcaagaaaatcgagtgcttcgactccgtggaaatctccggcgtggaagatcggttcaacgcctccctgggcacataccacgatctgctgaaaattatcaaggacaaggacttcctggacaatgaggaaaacgaggacattctggaagatatcgtgctgaccctgacactgtttgaggacagagagatgatcgaggaacggctgaaaacctatgcccacctgttcgacgacaaagtgatgaagcagctgaagcggcggagatacaccggctggggcaggctgagccggaagctgatcaacggcatccgggacaagcagtccggcaagacaatcctggatttcctgaagtccgacggcttcgccaacagaaacttcatgcagctgatccacgacgacagcctgacctttaaagaggacatccagaaagcccaggtgtccggccagggcgatagcctgcacgagcacattgccaatctggccggcagccccgccattaagaagggcatcctgcagacagtgaaggtggtggacgagctcgtgaaagtgatgggccggcacaagcccgagaacatcgtgatcgaaatggccagagagaaccagaccacccagaagggacagaagaacagccgcgagagaatgaagcggatcgaagagggcatcaaagagctgggcagccagatcctgaaagaacaccccgtggaaaacacccagctgcagaacgagaagctgtacctgtactacctgcagaatgggcgggatatgtacgtggaccaggaactggacatcaaccggctgtccgactacgatgtggacgctatcgtgcctcagagctttctgaaggacgactccatcgataacaaagtgctgactcggagcgacaagaaccggggcaagagcgacaacgtgccctccgaagaggtcgtgaagaagatgaagaactactggcgccagctgctgaatgccaagctgattacccagaggaagttcgacaatctgaccaaggccgagagaggcggcctgagcgaactggataaggccggcttcatcaagagacagctggtggaaacccggcagatcacaaagcacgtggcacagatcctggactcccggatgaacactaagtacgacgagaacgacaaactgatccgggaagtgaaagtgatcaccctgaagtccaagctggtgtccgatttccggaaggatttccagttttacaaagtgcgcgagatcaacaactaccaccacgcccacgacgcctacctgaacgccgtcgtgggaaccgccctgatcaaaaagtaccctaagctggaaagcgagttcgtgtacggcgactacaaggtgtacgacgtgcggaagatgatcgccaagagcgagcaggaaatcggcaaggctaccgccaagtacttcttctacagcaacatcatgaactttttcaagaccgagattaccctggccaacggcgagatccggaagcggcctctgatcgagacaaacggcgaaacaggcgagatcgtgtgggataagggccgggactttgccaccgtgcggaaagtgctgtctatgccccaagtgaatatcgtgaaaaagaccgaggtgcagacaggcggcttcagcaaagagtctatcctgcccaagaggaacagcgacaagctgatcgccagaaagaaggactgggaccctaagaagtacggcggcttcgacagccccaccgtggcctattctgtgctggtggtggccaaagtggaaaagggcaagtccaagaaactgaagagtgtgaaagagctgctggggatcaccatcatggaaagaagcagcttcgagaagaatcccatcgactttctggaagccaagggctacaaagaagtgaaaaaggacctgatcatcaagctgcctaagtactccctgttcgagctggaaaacggccggaagagaatgctggcctctgccggcgaactgcagaagggaaacgaactggccctgccctccaaatatgtgaacttcctgtacctggccagccactatgagaagctgaagggctcccccgaggataatgagcagaaacagctgtttgtggaacagcacaaacactacctggacgagatcatcgagcagatcagcgagttctccaagagagtgatcctggccgacgctaatctggacaaggtgctgagcgcctacaacaagcacagagacaagcctatcagagagcaggccgagaatatcatccacctgtttaccctgaccaatctgggagcccctgccgccttcaagtactttgacaccaccatcgaccggaagaggtacaccagcaccaaagaggtgctggacgccaccctgatccaccagagcatcaccggcctgtacgagacacggatcgacctgtctcagctgggaggcgacgcctatccctatgacgtgcccgattatgccagcctgggcagcggctcccccaagaaaaaacgcaaggtggaagatcctaagaaaaagcggaaagtggaagatgctccaaagaagaagagaaaggtcgacggcattggtagtgggagcaacggcagcagcggatcccttaagcttgccaccatgcgtacgggcaagaatgagcaggaactgctggaactggacaaatgggcaagcctggggagcggctccggaaagaacgagcaggaactgctggaactggacaagtgggcttctctgggctccgggtccgggaaaaacgaacaggagctgctggagctggataagtgggccagcctgggctccgggtccgggaagaatgaacaggaactgctggaactggataaatgggcatctctgggcagcgggtccggaaagaatgaacaggagctgctggaactggacaagtgggcatccctggggagcgggtccggaaagaatgaacaggaactgctggaactggataagtgggcatccctgggaagcggctccggaaaaaacgagcaggagctgctggagctggacaagtgggcctctctgggctccggctccgggaagaatgaacaggagctgctggaactggataagtgggcatctctggggtccggaagcggtaaaaacgagcaggaactgctggaactggacaagtgggcaagcctggggtctggctccggcaaaaacgaacaggaactgctggaactggacaaatgggccagcctgggctccgggtccgggaaaaatgagcaggaactgctggaactggataaatgggcgagcctgggcagcgggtccggaaaaaacgaacaggagctgctggagctggacaaatgggcgagtctgggctccggaagcggcaagaatgaacaggaactgctggagctggacaaatgggccagcctgggcagcggctccggaaagaatgagcaggaactgctggagctggacaaatgggcatctctgggctccggctccgggaaaaacgagcaggaactgctggagctggacaagtgggcttccctgggcagcggctccggaaaaaatgaacaggaactgctggaactggacaagtgggccagcctggggtccgggtctgggaagaacgagcaggaactgctggagctggacaagtgggcatctctggggtctggctctggcaagaacgagcaggaactgctggaactggacaagtgggcgagcctgggcagcgggtctggcaagaatgaacaggagctgctggaactggacaaatgggccagcctggggtctgggtctggaaagaatgagcaggaactgctggagctggacaagtgggcatctctgggctctgggtctggcaagaacgagcaggagctgctggagctggataagtgggcatccctggggtccgggtccggcaagaatgaacaggagctgctggagctggataaatgggcatccctgggctctgggtctgggaagaatgagcaggagctgctggaactggataagtgggcgagcctgggcagcgggtctggcaagaatgagcaggaactgctggagctggacaagtgggcttctctgcaccggtcagcagtggaggacagcacgtaacccgggcttatatgaagatgaagatgaaatatttggtgtgtcaaataaaaagcttgtgtgcttaagtttgtgtttttttcttggcttgttgtgttatgaatttgtggctttttctaatattaaatgaatgtaagatcacattataatgaataaacaaatgtttctataatccattgtgaatgttttgttggatctcttctgcagcatataactactgtatgtgctatggtatggactatggaatatgattaaagataagcgctgagctcggacggcgcgccggagtggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattggatttgcattaactcgggtgttttagagctagaaatagcaagttaaaataaggctagtccgttatcaacttgaaaaagtggcaccgagtcggtgctttttttcccggtgacgcaatggtctattggcgttgttgttttccattagggagcttatagattattgattaatatgatattgtggagccacgagtatcattgataagatataaatttggccaaaaatgcttttgattgaaatatgcaacaccattaagagtgtttttgtgaaaaaggaatgctcgtactgcaagctatggtttcttctattgtatgacattgccattacttgctatctctattgattcattttaagcactttcctgataaagaatctgttttatccggttgattgctcttatttgtctgttactgctcaaaccttacactatactcccaatgtttctatgccacaggttaatctaaagaggtgaccgaaagaagaaaacttttacgttttgcatggagtaagaatgccagagttaagaagtggagcaaggagatcgagacgccttgacgagcagcctaaccctccgctagttgaacaagcagaaaatatcgtacttcctcctcagactgcaacaaggagaagaggtggtggctttggcttcgttgaacaacggaaactcgacttgccttccgcacaatacatcatttcttcttagctttttttcttcttcttcgttcatacagtttttttttgtttatcagcttacattttcttgaaccgtagctttcgttttcttctttttaactttccattcggagtttttgtatcttgtttcatagtttgtcccaggattagaatgattaggcatcgaaccttcaagaatttgattgaataaaacatcttcattcttaagatatgaagataatcttcaaaaggcccctgggaatctgaaagaagagaagcaggcccatttatatgggaaagaacaatagtatttcttatataggcccatttaagttgaaaacaatcttcaaaagtcccacatcgcttagataagaaaacgaagctgagtttatatacagctagagtcgaagtagtgattgtccagtgtattagtgtggtgttttagagctagaaatagcaagttaaaataaggctagtccgttatcaacttgaaaaagtggcaccgagtcggtgctttttttcccggtgacgccgctgagctcccggggcgcgccctatggagttctagaatgtcgcggaacaaattttaaaactaaatcctaaatttttctaattttgttgccaatagtggatatgtgggccgtatagaaggaatctattgaaggcccaaacccatactgacgagcccaaaggttcgttttgcgttttatgtttcggttcgatgccaacgccacattctgagctaggcaaaaaacaaacgtgtctttgaatagactcctctcgttaacacatgcagcggctgcatggtgacgccattaacacgtggcctacaattgcatgatgtctccattgacacgtgacttctcgtctcctttcttaatatatctaacaaacactcctacctcttccaaaatatatacacatctttttgatcaatctctcattcaaaatctcattctctctagtaaacaagaacaaaaaaatggcggatacagctagaggaacccatcacgatatcatcggcagagatcagtacccgatgatgggccgagatcgtgaccagtaccagatgtccggacgaggatctgactactccaagtctaggcagattgctaaagctgcaactgctgtcacagctggtggttccctccttgttctctccagccttacccttgttggaactgtcatagctttgactgttgcaacacctctgctcgttatcttcagcccaatccttgtcccggctctcatcacagttgcactcctcatcaccggttttctttcctctggagggtttggcattgccgctataaccgttttctcttggatttacaagtaagcacacatttatcatcttacttcataattttgtgcaatatgtgcatgcatgtgttgagccagtagctttggatcaatttttttggtcgaataacaaatgtaacaataagaaattgcaaattctagggaacatttggttaactaaatacgaaatttgacctagctagcttgaatgtgtctgtgtatatcatctatataggtaaaatgcttggtatgatacctattgattgtgaataggtacgcaacgggagagcacccacagggatcagacaagttggacagtgcaaggatgaagttgggaagcaaagctcaggatctgaaagacagagctcagtactacggacagcaacatactggtggggaacatgaccgtgaccgtactcgtggtggccagcacactactatgagcgagctgattaaggagaacatgcacatgaagctgtacatggagggcaccgtgaacaaccaccacttcaagtgcacatccgagggcgaaggcaagccctacgagggcacccagaccatgagaatcaaggtggtcgagggcggccctctccccttcgccttcgacatcctggctaccagcttcatgtacggcagcagaaccttcatcaaccacacccagggcatccccgacttctttaagcagtccttccctgagggcttcacatgggagagagtcaccacatacgaagatgggggcgtgctgaccgctacccaggacaccagcctccaggacggctgcctcatctacaacgtcaagatcagaggggtgaacttcccatccaacggccctgtgatgcagaagaaaacactcggctgggaggccaacaccgagatgctgtaccccgctgacggcggcctggaaggcagaagcgacatggccctgaagctcgtgggcgggggccacctgatctgcaacttcaagaccacatacagatccaagaaacccgctaagaacctcaagatgcccggcgtctactatgtggaccacagactggaaagaatcaaggaggccgacaaagaaacctacgtcgagcagcacgaggtggctgtggccagatactgcgacctccctagcaaactggggcacaagtgagcttaccccactgatgtcatcgtcatagtccaataactccaatgtcggggagttagtttatgaggaataaagtgtttagaatttgatcagggggagataataaaagccgagtttgaatctttttgttataagtaatgtttatgtgtgtttctatatgttgtcaaatggtaccatgttttttttcctctctttttgtaacttgcaagtgttgtgttgtactttatttggcttctttgtaagttggtaacggtggtctatatatggaaaaggtcttgttttgttaaacttatgttagttaactggattcgtctttaaccacaaaaagttttcaataagctacaaatttagacacgcaagccgatgcagtcattagtacatatatttattgcaagtgattacatggcaacccaaacttcaaaaacagtaggttgctccatttagtcgctgttagagctctcccggcgcgccggaggtcgagctgcaggtcaacggatcaggatattcttgtttaagatgttgaactctatggaggtttgtatgaactgatgatctaggaccggataagttcccttcttcatagcgaacttattcaaagaatgttttgtgtatcattcttgttacattgttattaatgaaaaaatattattggtcattggactgaacacgagtgttaaatatggaccaggccccaaataagatccattgatatatgaattaaataacaagaataaatcgagtcaccaaaccacttgccttttttaacgagacttgttcaccaacttgatacaaaagtcattatcctatgcaaatcaataatcatacaaaaatatccaataacactaaaaaattaaaagaaatggataatttcacaatatgttatacgataaagaagttacttttccaagaaattcactgattttataagcccacttgcattagataaatggcaaaaaaaaacaaaaaggaaaagaaataaagcacgaagaattctagaaaatacgaaatacgcttcaatgcagtgggacccacggttcaattattgccaattttcagctccaccgtatatttaaaaaataaaacgataatgctaaaaaaatataaatcgtaacgatcgttaaatctcaacggctggatcttatgacgaccgttagaaattgtggttgtcgacgagtcagtaataaacggcgtcaaagtggttgcagccggcacacacgagtcgtgtttatcaactcaaagcacaaatacttttcctcaacctaaaaataaggcaattagccaaaaacaactttgcgtgtaaacaacgctcaatacacgtgtcattttattattagctattgcttcaccgccttagctttctcgtgacctagtcgtcctcgtcttttcttcttcttcttctataaaacaatacccaaagagctcttcttcttcacaattcagatttcaatttctcaaaatcttaaaaactttctctcaattctctctaccgtgatcaaggtaaatttctgtgttccttattctctcaaaatcttcgattttgttttcgttcgatcccaatttcgtatatgttctttggtttagattctgttaatcttagatcgaacacgattttctgggtttgatcgttagatatcatcttaattctcgattagggtttcatagatatcatccgatttgttcaaataatttgagttttgtcgaataattactcttcgatttgtgatttctatctagatctggtgttagtttctagtttgtgcgatcgaatttgtcgattaatctgagtttttctgattaacaggaatgccaaagaagaagaggaaagtcatggaggtgcagctggtggaatctgggggcggactggtgcagcccgggggatctctgcggctgtcctgcgccgcctctggctccatctctagcgtggatgtgatgtcctggtacaggcaggcccctggcaagcagagggaactggtggccttcattactgataggggaagaaccaattacaaagtgagcgtgaaaggccgcttcactatcagccgggataattctaagaatatggtgtatctgcagatgaacagcctgaagccagaagacactgccgactatctgtgcagggctgagtcccggacttcctggtccagcccatctcctctggatgtgtgggggcgcggcactcaggtgactgtgtcctccttggatccaggtggaggtggaagcggtagcaaaggagaagaacttttcactggagttgtcccaattcttgttgaattagatggtgatgttaatgggcacaaattttctgtccgtggagagggtgaaggtgatgctacaaacggaaaactcacccttaaatttatttgcactactggaaaactacctgttccgtggccaacacttgtcactactctgacctatggtgttcaatgcttttcccgttatccggatcacatgaaacggcatgactttttcaagagtgccatgcccgaaggttatgtacaggaacgcactatatctttcaaagatgacgggacctacaagacgcgtgctgaagtcaagtttgaaggtgatacccttgttaatcgtatcgagttaaagggtattgattttaaagaagatggaaacattcttggacacaaactcgagtacaactttaactcacacaatgtatacatcacggcagacaaacaaaagaatggaatcaaagctaacttcaaaattcgccacaacgttgaagatggttccgttcaactagcagaccattatcaacaaaatactccaattggcgatggccctgtccttttaccagacaaccattacctgtcgacacaatctgtcctttcgaaagatcccaacgaaaagcgtgaccacatggtccttcttgagtttgtaactgctgctgggattacacatggcatggatgagctctacaaaggtagtggcggtggtggttcaggaggcggcggaagcttagatccaggtggaggtggaggtagcagcagcgatatgtttgacgtagatgagctgctaagagatctcaacggggatgatgttttcgccggcctcaaccaggacagatacccgggcaatagcgtggcgaatggctcctacaggcctgagtcacaacagtcagggtttgacccacttcagtctcttaattacggtatcccacctttccagctcgaaggtaaagacgggaacggattcttcgacgacctatcttacttggatcttgagaacggaggtggtagtggtggaggtcggaccgaagagtacaagcttatcctgaacggtaaaaccctgaaaggtgaaaccaccaccgaagctgttgacgctgctaccgcggaaaaagttttcaaacagtacgctaacgacaacggtgttgacggtgaatggacctacgacgacgctaccaaaaccttcacggtaaccgaataagcttgttcgagtattatggcattgggaaaactgtttttcttgtaccatttgttgtgcttgtaatttactgtgttttttattcggttttcgctatcgaactgtgaaatggaaatggatggagaagagttaatgaatgatatggtccttttgttcattctcaaattaatattatttgttttttctcttatttgttgtgtgttgaatttgaaattataagagatatgcaaacattttgttttgagtaaaaatgtgtcaaatcgtggcctctaatgaccgaagttaatatgaggagtaaaacacttgtagttgtcgctgagctcagtg

SEQ ID NO: 119 - protein, 1368 aa, synthetic construct
MDKKYSIGLAIGTNSVGWAVITDEYKVPSKKFKVLGNTDRHSIKKNLIGALLFDSGETAEATRLKRTARRRYTRRKNRICYLQEIFSNEMAKVDDSFFHRLEESFLVEEDKKHERHPIFGNIVDEVAYHEKYPTIYHLRKKLVDSTDKADLRLIYLALAHMIKFRGHFLIEGDLNPDNSDVDKLFIQLVQTYNQLFEENPINASGVDAKAILSARLSKSRRLENLIAQLPGEKKNGLFGNLIALSLGLTPNFKSNFDLAEDAKLQLSKDTYDDDLDNLLAQIGDQYADLFLAAKNLSDAILLSDILRVNTEITKAPLSASMIKRYDEHHQDLTLLKALVRQQLPEKYKEIFFDQSKNGYAGYIDGGASQEEFYKFIKPILEKMDGTEELLVKLNREDLLRKQRTFDNGSIPHQIHLGELHAILRRQEDFYPFLKDNREKIEKILTFRIPYYVGPLARGNSRFAWMTRKSEETITPWNFEEVVDKGASAQSFIERMTNFDKNLPNEKVLPKHSLLYEYFTVYNELTKVKYVTEGMRKPAFLSGEQKKAIVDLLFKTNRKVTVKQLKEDYFKKIECFDSVEISGVEDRFNASLGTYHDLLKIIKDKDFLDNEENEDILEDIVLTLTLFEDREMIEERLKTYAHLFDDKVMKQLKRRRYTGWGRLSRKLINGIRDKQSGKTILDFLKSDGFANRNFMQLIHDDSLTFKEDIQKAQVSGQGDSLHEHIANLAGSPAIKKGILQTVKVVDELVKVMGRHKPENIVIEMARENQTTQKGQKNSRERMKRIEEGIKELGSQILKEHPVENTQLQNEKLYLYYLQNGRDMYVDQELDINRLSDYDVDAIVPQSFLKDDSIDNKVLTRSDKNRGKSDNVPSEEVVKKMKNYWRQLLNAKLITQRKFDNLTKAERGGLSELDKAGFIKRQLVETRQITKHVAQILDSRMNTKYDENDKLIREVKVITLKSKLVSDFRKDFQFYKVREINNYHHAHDAYLNAVVGTALIKKYPKLESEFVYGDYKVYDVRKMIAKSEQEIGKATAKYFFYSNIMNFFKTEITLANGEIRKRPLIETNGETGEIVWDKGRDFATVRKVLSMPQVNIVKKTEVQTGGFSKESILPKRNSDKLIARKKDWDPKKYGGFDSPTVAYSVLVVAKVEKGKSKKLKSVKELLGITIMERSSFEKNPIDFLEAKGYKEVKKDLIIKLPKYSLFELENGRKRMLASAGELQKGNELALPSKYVNFLYLASHYEKLKGSPEDNEQKQLFVEQHKHYLDEIIEQISEFSKRVILADANLDKVLSAYNKHRDKPIREQAENIIHLFTLTNLGAPAAFKYFDTTIDRKRYTSTKEVLDATLIHQSITGLYETRIDLSQLGGD

SEQ ID NO: 120 - protein, 15 aa, synthetic construct
KNEQELLELDKWASL

SEQ ID NO: 121 - protein, 19 aa, Saccharomyces cerevisiae
EELLSKNYHLENEVARLKK

SEQ ID NO: 122 - protein, 123 aa, synthetic construct
MEVQLVESGGGLVQPGGSLRLSCAASGSISSVDVMSWYRQAPGKQRELVAFITDRGRTNYKVSVKGRFTISRDNSKNMVYLQMNSLKPEDTADYLCRAESRTSWSSPSPLDVWGRGTQVTVSS

SEQ ID NO: 123 - protein, 284 aa, synthetic construct
MGPDIVMTQSPSSLSASVGDRVTITCRSSTGAVTTSNYASWVQEKPGKLFKGLIGGTNNRAPGVPSRFSGSLIGDKATLTISSLQPEDFATYFCALWYSNHWVFGQGTKVELKRGGGGSGGGGSGGGGSSGGGSEVKLLESGGGLVQPGGSLKLSCAVSGFSLTDYGVNWVRQAPGRGLEWIGVIWGDGITDYNSALKDRFIISKDNGKNTVYLQMSKVRSDDTALYYCVTGLFDYWGQGTLVTVSSYPYDVPDYAGGGGGSGGGGSGGGGSGGGGSLDPGGGG

SEQ ID NO: 124 - protein, 5 aa, synthetic construct
GSGSG

SEQ ID NO: 125 - protein, 54 aa, synthetic construct
YKLILNGKTLKGETTTEAVDAATAEKVFKQYANDNGVDGEWTYDDATKTFTVTE

SEQ ID NO: 126 - protein, 237 aa, synthetic construct
SKGEELFTGVVPILVELDGDVNGHKFSVRGEGEGDATNGKLTLKFICTTGKLPVPWPTLVTTLTYGVQCFSRYPDHMKRHDFFKSAMPEGYVQERTISFKDDGTYKTRAEVKFEGDTLVNRIELKGIDFKEDGNILGHKLEYNFNSHNVYITADKQKNGIKANFKIRHNVEDGSVQLADHYQQNTPIGDGPVLLPDNHYLSTQSVLSKDPNEKRDHMVLLEFVTAAGITHGMDELYK

SEQ ID NO: 127 - protein, 220 aa, Escherichia coli
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSD

SEQ ID NO: 128 - protein, 109 aa, Escherichia coli
MSDKIIHLTDDSFDTDVLKADGAILVDFWAEWCGPCKMIAPILDEIADEYQGKLTVAKLNIDQNPGTAPKYGIRGIPTLLLFKNGEVAATKVGALSKGQLKEFLDANLA

SEQ ID NO: 129 - protein, 99 aa, Homo sapiens
GSMSDQEAKPSTEDLGDKKEGEYIKLKVIGQDSSEIHFKVKMTTHLKKLKESYCQRQGVPMNSLRFLFEGQRIADNHTPKELGMEEEDVIEVYQEQTGG

SEQ ID NO: 130 - protein, 392 aa, Escherichia coli
MKIKTGARILALSALTTMMFSASALAKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQT

SEQ ID NO: 131 - protein, 8 aa, synthetic construct
DYKDDDDK

SEQ ID NO: 132 - DNA, 20 bp, synthetic construct
  source 1..20: mol_type = other DNA
gttggaggaagagtcggctg

SEQ ID NO: 133 - DNA, 21 bp, synthetic construct
  source 1..21: mol_type = other DNA
tgcttctcgtcgacaagtctc

SEQ ID NO: 134 - DNA, 20 bp, synthetic construct
  source 1..20: mol_type = other DNA
acaagaatgccttccaagcc

SEQ ID NO: 135 - DNA, 20 bp, synthetic construct
  source 1..20: mol_type = other DNA
cggtcttgtccttcacgaag

SEQ ID NO: 136 - DNA, 20 bp, synthetic construct
  source 1..20: mol_type = other DNA
tggtgaaaccagaggcaagg

SEQ ID NO: 137 - DNA, 20 bp, synthetic construct
  source 1..20: mol_type = other DNA
cgtttgtgcagcttgagacg

SEQ ID NO: 138 - DNA, 23 bp, synthetic construct
  source 1..23: mol_type = other DNA
caagaagactaaacacgaaagcg

SEQ ID NO: 139 - DNA, 22 bp, synthetic construct
  source 1..22: mol_type = other DNA
acagtagtaccgtcacctctac

SEQ ID NO: 140 - DNA, 20 bp, synthetic construct
  source 1..20: mol_type = other DNA
catggctgctaagagcaacg

SEQ ID NO: 141 - DNA, 23 bp, synthetic construct
  source 1..23: mol_type = other DNA
agatgaaattctcagctgcttgc

SEQ ID NO: 142 - DNA, 19 bp, synthetic construct
  source 1..19: mol_type = other DNA
gcctgccagagacgatctc

SEQ ID NO: 143 - DNA, 20 bp, synthetic construct
  source 1..20: mol_type = other DNA
gtattgctgttctccggtgc

SEQ ID NO: 144 - DNA, 20 bp, synthetic construct
  source 1..20: mol_type = other DNA
ttcaaggactttccaaccgc

SEQ ID NO: 145 - DNA, 21 bp, synthetic construct
  source 1..21: mol_type = other DNA
cttggtgggttcacatgatgg

SEQ ID NO: 146 - DNA, 21 bp, synthetic construct
  source 1..21: mol_type = other DNA
atctgaaaggctttcaccagc

SEQ ID NO: 147 - DNA, 21 bp, synthetic construct
  source 1..21: mol_type = other DNA
aagaagtccaggagggtcaag

SEQ ID NO: 148 - DNA, 19 bp, synthetic construct
  source 1..19: mol_type = other DNA
aacgtggccaaaatgatgc

SEQ ID NO: 149 - DNA, 20 bp, synthetic construct
  source 1..20: mol_type = other DNA
aaccgcttggtcgactatcg